SEQUENCE LISTING (123 sequences)

SEQ ID NO: 1 - protein, 10 aa, synthetic construct
MIHYLGWSKV

SEQ ID NO: 2 - protein, 10 aa, synthetic construct
YMGSRQNDMG

SEQ ID NO: 3 - protein, 10 aa, synthetic construct
LNRAPYFPNA

SEQ ID NO: 4 - protein, 9 aa, synthetic construct
WLSNNFWAW

SEQ ID NO: 5 - protein, 10 aa, synthetic construct
QIKMWWAMMH

SEQ ID NO: 6 - protein, 10 aa, synthetic construct
TSFWIKARKW

SEQ ID NO: 7 - protein, 10 aa, synthetic construct
WASWRWFRDH

SEQ ID NO: 8 - protein, 10 aa, synthetic construct
QTLKTSWWWT

SEQ ID NO: 9 - protein, 18 aa, synthetic construct
LPGGNNFNLYRSFIMTLW

SEQ ID NO: 10 - protein, 41 aa, synthetic construct
WQLGSGGLHVWVSCHMYGGVYGKTSELALTWPGMWTRTHLW

SEQ ID NO: 11 - protein, 18 aa, synthetic construct
DSPRLDSIWSMSHAMGHL

SEQ ID NO: 12 - protein, 40 aa, synthetic construct
YLVRFRLDGVVWHEVVGNVNEYRTAWASIFDWLAKFQLGS

SEQ ID NO: 13 - protein, 40 aa, synthetic construct
ILNSCNPTWTRLQFKFCPALLMNGPIFAWFLWAAWVSSAM

SEQ ID NO: 14 - protein, 40 aa, synthetic construct
SDEYRFDRSVESVWQDLVIYQSHVLWWTTVHRYLAAWIQW

SEQ ID NO: 15 - protein, 18 aa, synthetic construct
VNWINMHSAKWMWWTHGV

SEQ ID NO: 16 - protein, 18 aa, synthetic construct
TPTLNYGNFNERDMDTVG

SEQ ID NO: 17 - protein, 40 aa, synthetic construct
FTHISRFGEQHPTLVNLFYPRWQYIFIFTRNALCGQYVWD

SEQ ID NO: 18 - protein, 40 aa, synthetic construct
LSDFEINKRCVLGVFYYLGEGSRGHILSFFHPNVQQFRVP

SEQ ID NO: 19 - protein, 40 aa, synthetic construct
GDFGNRYIQLLVIVLSNGQIFVQKLLSMRPFTINGDRLRQ

SEQ ID NO: 20 - protein, 40 aa, synthetic construct
ASVTLFRLVPMQNAWVTVLPSFAEFVMARPIEERATWGGS

SEQ ID NO: 21 - protein, 40 aa, synthetic construct
WQLGSGGLHVWVSCHMYGGVYGKTSELALTWPGMWTRTHL

SEQ ID NO: 22 - protein, 40 aa, synthetic construct
DSTMTLLARANSHASLLNVYLNINQKVEDVFFMLFLGSPP

SEQ ID NO: 23 - protein, 40 aa, synthetic construct
LATWIQGSKIWSNETYSISIQVSWLSLFVMGQNLYYLLSV

SEQ ID NO: 24 - protein, 40 aa, synthetic construct
QKVLYTAVPFKHGSYIFCDRIGFIFNYICEFSRSWSTWSM

SEQ ID NO: 25 - protein, 40 aa, synthetic construct
VGNSIQYVKAVQTQYWAAITSWIPITRFTTRVMFLLPQVM

SEQ ID NO: 26 - protein, 40 aa, synthetic construct
QVTARCLQIIVLYKLGNNGVTSLFHVYAMSSVPKFSVKWS

SEQ ID NO: 27 - protein, 40 aa, synthetic construct
GQVCSPFVTSALKLFGADFGWWSLVLMKAVSWVLYITFNA

SEQ ID NO: 28 - protein, 40 aa, synthetic construct
TLDHVRQAYAALSYSTVRCTFVSQMTIFYFGRVMFKALCN

SEQ ID NO: 29 - protein, 40 aa, synthetic construct
AYHLQLEQLRCVSQNVVTSFLDFALTKALALRSVLLGVWM

SEQ ID NO: 30 - protein, 40 aa, synthetic construct
QCFQDDLCVLPVASLLDHTRSMSHLFEGWLPSRSFGFRNG

SEQ ID NO: 31 - protein, 40 aa, synthetic construct
STAVVIWQDWFRTFRMAVVVSSARDEAVDRVCLAWPARYM

SEQ ID NO: 32 - protein, 40 aa, synthetic construct
LSAADMYKHKIVGEAGEPIFYMVRNWLQKQWPMNFILMSL

SEQ ID NO: 33 - protein, 40 aa, synthetic construct
FLWQWSADSMGAVDACSHKILGLFYHSQNIFARPFLRSYG

SEQ ID NO: 34 - protein, 40 aa, synthetic construct
NIFIPPVGYYLTKQCGTNNFWFYQVLCRRWGADSILFWER

SEQ ID NO: 35 - protein, 134 aa, synthetic construct
MAQVQLVESGGGVVQPGRSLRLSCAASMIHYLGWSKVWFRQAPGKEREFVAQIKMWWAMMHYADSVKGRFTISRDNSKNTLYLQMNSLRAEDTAVYYCARLPGGNNFNLYRSFIMTLWWGQGTLVTVSSGPGGQ

SEQ ID NO: 36 - protein, 157 aa, synthetic construct
MAQVQLVESGGGVVQPGRSLRLSCAASMIHYLGWSKVWFRQAPGKEREFVAQIKMWWAMMHYADSVKGRFTISRDNSKNTLYLQMNSLRAEDTAVYYCARWQLGSGGLHVWVSCHMYGGVYGKTSELALTWPGMWTRTHLWWGQGTLVTVSSGPGGQ

SEQ ID NO: 37 - protein, 157 aa, synthetic construct; identical to SEQ ID NO: 36

SEQ ID NO: 38 - protein, 156 aa, synthetic construct
MAQVQLVESGGGVVQPGRSLRLSCAASYMGSRQNDMGWFRQAPGKEREFVATSFWIKARKWYADSVKGRFTISRDNSKNTLYLQMNSLRAEDTAVYYCARYLVRFRLDGVVWHEVVGNVNEYRTAWASIFDWLAKFQLGSWGQGTLVTVSSGPGGQ

SEQ ID NO: 39 - protein, 155 aa, synthetic construct
MQVQLVESGGGVVQPGRSLRLSCAASLNRAPYFPNAWFRQAPGKEREFVAWASWRWFRDHYADSVKGRFTISRDNSKNTLYLQMNSLRAEDTAVYYCARILNSCNPTWTRLQFKFCPALLMNGPIFAWFLWAAWVSSAMWGQGTLVTVSSGPGGQ

SEQ ID NO: 40 - protein, 155 aa, synthetic construct
MQVQLVESGGGVVQPGRSLRLSCAASSWLSNNFWAWWFRQAPGKEREFVAQTLKTSWWWTYADSVKGRFTISRDNSKNTLYLQMNSLRAEDTAVYYCARSDEYRFDRSVESVWQDLVIYQSHVLWWTTVHRYLAAWIQWWGQGTLVTVSSGPGGQ

SEQ ID NO: 41 - protein, 133 aa, synthetic construct
MQVQLVESGGGVVQPGRSLRLSCAASLNRAPYFPNAWFRQAPGKEREFVAWASWRWFRDHYADSVKGRFTISRDNSKNTLYLQMNSLRAEDTAVYYCARVNWINMHSAKWMWWTHGVWGQGTLVTVSSGPGGQ

SEQ ID NO: 42 - protein, 133 aa, synthetic construct
MQVQLVESGGGVVQPGRSLRLSCAASSWLSNNFWAWWFRQAPGKEREFVAQTLKTSWWWTYADSVKGRFTISRDNSKNTLYLQMNSLRAEDTAVYYCARTPTLNYGNFNERDMDTVGWGQGTLVTVSSGPGGQ

SEQ ID NO: 43 - protein, 40 aa, synthetic construct
YQVVSVVWHRGLLRPVLRGSLKPWYIFVRIHISWGQVHVG

SEQ ID NO: 44 - protein, 40 aa, synthetic construct
TGWWFFWSYVCSVRLLAVTCSFWCTEQGEESLPRAGTHTH

SEQ ID NO: 45 - protein, 40 aa, synthetic construct
YTLGLGKSQRWNLFRNFFCYMPRVTVFCKSCRCVSFQTSG

SEQ ID NO: 46 - protein, 40 aa, synthetic construct
HYLLIRCARGFCEVRLVDAMCSITIVIDCVLFTSQRERAE

SEQ ID NO: 47 - protein, 40 aa, synthetic construct
QGSLISCWVTFVQESWDAYSLCSDLFKCQGGQVFSICEPE

SEQ ID NO: 48 - protein, 25 aa, synthetic construct
DFYGCLLDLSLGVPSLGWRRRCITA

SEQ ID NO: 49 - protein, 26 aa, synthetic construct
FSFGNSSLRKLPGVVRRRRCARGVWR

SEQ ID NO: 50 - protein, 20 aa, synthetic construct
PRGWRRCLGWSVGPASGSGP

SEQ ID NO: 51 - protein, 20 aa, synthetic construct
CLGVCISGDPEQADMWGCRP

SEQ ID NO: 52 - protein, 20 aa, synthetic construct
ILSRSQYLVFGYAVVARGSR

SEQ ID NO: 53 - protein, 25 aa, synthetic construct
GSGLCSVCQCGGMYSRVVLGQRRWM

SEQ ID NO: 54 - protein, 20 aa, synthetic construct
GVSPIVHGDAGLLQPRNTRL

SEQ ID NO: 55 - protein, 20 aa, synthetic construct
CRQRGDGGAMRRGSVCLRGW

SEQ ID NO: 56 - protein, 20 aa, synthetic construct
GDGTGVQRAAVGFDLTGHSP

SEQ ID NO: 57 - protein, 25 aa, synthetic construct
AVSACGRCRHCRVPGVPCWLYCVCT

SEQ ID NO: 58 - protein, 20 aa, synthetic construct
PSTMSIAGGGFCGHPRRGTI

SEQ ID NO: 59 - protein, 20 aa, synthetic construct
DQCAGHDSVQRHALHGLAVM

SEQ ID NO: 60 - protein, 25 aa, synthetic construct
GASPCVFRVDSTSGGPLVFPDLQST

SEQ ID NO: 61 - protein, 20 aa, synthetic construct
RREDSLTRVRQALREALGAG

SEQ ID NO: 62 - protein, 20 aa, synthetic construct
LDVGVAQHRGGTVIRVAMRA

SEQ ID NO: 63 - protein, 20 aa, synthetic construct
GIRRGGLGRQRGWGRRRKPI

SEQ ID NO: 64 - protein, 19 aa, synthetic construct
RAPGGFTRTNSVRRATGSM

SEQ ID NO: 65 - protein, 20 aa, synthetic construct
YGKVGGWRLATVGRRTESRI

SEQ ID NO: 66 - protein, 20 aa, synthetic construct
KQVGLINGSACSCDMYQRVV

SEQ ID NO: 67 - protein, 20 aa, synthetic construct
VNSVKGVHRRAFSEQPLGSG

SEQ ID NO: 68 - protein, 20 aa, synthetic construct
DAGRAPALGWFFFGRRRFGE

SEQ ID NO: 69 - protein, 25 aa, synthetic construct
GVPGCGSHGADWGREIGATSTGMAL

SEQ ID NO: 70 - protein, 20 aa, synthetic construct
GVCRWMRPHFRMPRRWRLGR

SEQ ID NO: 71 - protein, 20 aa, synthetic construct
RSSGMCIIARRHVLSCHSLS

SEQ ID NO: 72 - protein, 20 aa, synthetic construct
SGVGTCPTRPWVPGVCRQVS

SEQ ID NO: 73 - protein, 20 aa, synthetic construct
RVIVSGLSGRVRMGVKRRRI

SEQ ID NO: 74 - protein, 19 aa, synthetic construct
FMRHARPTRGVVRRIGHGV

SEQ ID NO: 75 - protein, 24 aa, synthetic construct
QLYACLRLVWGRQASAGWRVTMTG

SEQ ID NO: 76 - protein, 20 aa, synthetic construct
LERIVGQWYSEGLSGDWANL

SEQ ID NO: 77 - protein, 24 aa, synthetic construct
GASRCFSLCSGSEGAESRRQATTV

SEQ ID NO: 78 - protein, 19 aa, synthetic construct
HLGCSMPINVFGVCIWKCA

SEQ ID NO: 79 - protein, 20 aa, synthetic construct
RRLAVRLRVMLRRGPVCIRV

SEQ ID NO: 80 - protein, 24 aa, synthetic construct
LVDACIGSCQPEGSPPARPPFQLA

SEQ ID NO: 81 - protein, 26 aa, synthetic construct
RCVGCCKYLIAQAYRFTVAGGAAQYA

SEQ ID NO: 82 - protein, 23 aa, synthetic construct
AGVHCGLTNVHGVGVRSYRRLYA

SEQ ID NO: 83 - protein, 20 aa, synthetic construct
VNGWGLGRRGMGIDYRWAVH

SEQ ID NO: 84 - protein, 25 aa, synthetic construct
QSARCAVTMGLDALNLRRGISPVLD

SEQ ID NO: 85 - protein, 20 aa, synthetic construct
YPWQQVLVPPPGNVHAFATI

SEQ ID NO: 86 - protein, 20 aa, synthetic construct
TAVAGVGVSAGQLQGGTAAG

SEQ ID NO: 87 - protein, 20 aa, synthetic construct
FFGLSFPASVAVSDSGVQMR

SEQ ID NO: 88 - protein, 20 aa, synthetic construct
SSRAVSWMVQLRVQRGSGLV

SEQ ID NO: 89 - protein, 20 aa, synthetic construct
SRLSLVQSCSVSWGHQTVGT

SEQ ID NO: 90 - protein, 20 aa, synthetic construct
VARWCQVNWAYGRRLAPPVG

SEQ ID NO: 91 - protein, 20 aa, synthetic construct
FLLCSLPLTQPPYRVIGAYA

SEQ ID NO: 92 - protein, 20 aa, synthetic construct
GGPSGIGWQHAWWVFGRQGL

SEQ ID NO: 93 - protein, 20 aa, synthetic construct
LQRQIAGPRSRQRGRGSRYR

SEQ ID NO: 94 - protein, 20 aa, synthetic construct
FEDMRLGWRNGQPPGGVAIW

SEQ ID NO: 95 - protein, 20 aa, synthetic construct
DSWQGARVQPGVGAHTAQQR

SEQ ID NO: 96 - protein, 20 aa, synthetic construct
GIKTVRGYGVWRWHGGSPRM

SEQ ID NO: 97 - protein, 20 aa, synthetic construct
IRVWCQWLRTKLAAGQVNCA

SEQ ID NO: 98 - protein, 20 aa, synthetic construct
SYGVGGRHSGGQGEALVYSV

SEQ ID NO: 99 - protein, 20 aa, synthetic construct
RAQNCGNWAAVQRINVSSMA

SEQ ID NO: 100 - protein, 20 aa, synthetic construct
GRAPEQTPCIGWRGGLIKDL

SEQ ID NO: 101 - protein, 20 aa, synthetic construct
SGSRNFAAARWSHRGFCVPW

SEQ ID NO: 102 - protein, 20 aa, synthetic construct
IAFSRRGRGWWCQMPGGSRV

SEQ ID NO: 103 - protein, 20 aa, synthetic construct
GCCSAGLPSSLLSTPQSRSR

SEQ ID NO: 104 - protein, 24 aa, synthetic construct
DFYGCLLDLSLGVPSGVRRRCITA

SEQ ID NO: 105 - protein, 40 aa, synthetic construct
RPWILHLGRERSTDDQTPVHRMSLMHPLYHTRGKSYKVEG

SEQ ID NO: 106 - protein, 7 aa, synthetic construct
PKKKRKV

SEQ ID NO: 107 - protein, 6 aa, synthetic construct
WLPSSI

SEQ ID NO: 108 - protein, 6 aa, synthetic construct
PRRARS

SEQ ID NO: 109 - protein, 8 aa, synthetic construct
DYKDDDDK

SEQ ID NO: 110 - protein, 78 aa, synthetic construct
DFYGCLLDLSLGVPSLGWRRRCITADYKDDDDKWLPSSIPKKKRKVDGQKLRDAFTWNMNEKLMTPEMFSEILCDDLD

SEQ ID NO: 111 - protein, 70 aa, synthetic construct
DFYGCLLDLSLGVPSLGWRRRCITAWLPSSIPKKKRKVDGQKLRDAFTWNMNEKLMTPEMFSEILCDDLD

SEQ ID NO: 112 - protein, 183 aa, synthetic construct
QVQLVESGGGVVQPGRSLRLSCAASGGGGSGGGGSWFRQAPGKEREFVAGGGGSGGGGSYADSVKGRFTISRDNSKNTLYLQMNSLRAEDTAVYYCARDFYGCLLDLSLGVPSLGWRRRCITAWGQGLVTVSSGPGGQWLPSSIPKKKRKVDGQKLRDAFTWNMNEKLMTPEMFSEILCDDLD

SEQ ID NO: 113 - protein, 46 aa, synthetic construct
DFYGCLLDLSLGVPSLGWRRRCITADYKDDDDKWLPSSIPKKKRKV

SEQ ID NO: 114 - protein, 47 aa, synthetic construct
FSFGNSSLRKLPGVVRRRRCARGVWRDYKDDDDKWLPSSIPKKKRKV

SEQ ID NO: 115 - protein, 46 aa, synthetic construct
DFYGCLLDLSLGVPSLGWRRRCITADYKDDDDKPRRARSPKKKRKV

SEQ ID NO: 116 - protein, 47 aa, synthetic construct
FSFGNSSLRKLPGVVRRRRCARGVWRDYKDDDDKPRRARSPKKKRKV

SEQ ID NO: 117 - protein, 70 aa, synthetic construct
RPWILHLGRERSTDDQTPVHRMSLMHPLYHTRGKSYKVEGWLPSSIDFYGCLLDLSLGVPSGVRRRCITA

SEQ ID NO: 118 - protein, 10 aa, synthetic construct
GGSGGSGGSG

SEQ ID NO: 119 - protein, 72 aa, synthetic construct
RPWILHLGRERSTDDQTPVHRMSLMHPLYHTRGKSYKVEGWLPSSIFSFGNSSLRKLPGVVRRRRCARGVWR

SEQ ID NO: 120 - protein, 40 aa, synthetic construct
CVLGDYHYLYTGSADHTLRESLSLLTRCRFRRVIYQQQLC

SEQ ID NO: 121 - protein, 70 aa, synthetic construct
DFYGCLLDLSLGVPSLGWRRRCITAWLPSSIPKKKRKVDGQKLRDAFTWNMNEKLMTPEMFSEILADDLD

SEQ ID NO: 122 - protein, 385 aa, synthetic construct
MMMMALSKTFGQKPVKFQLEDDGEFYMIGSEVGNYLRMFRGSLYKRYPSLWRRLATVEERKKIVASSHGKKTKPNTKDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAW

SEQ ID NO: 123 - protein, 60 aa, synthetic construct
LVPIRLDMEIDGQKLRDAFTWNMNEKLMTPEMFSEILCDDLDLNPLTFVPAIASAIRQQI

(19) United States
(12) Patent Application Publication
(10) Pub. No.: US 2025/0082769 A1
(43) Pub. Date: Mar. 13, 2025

(54) CELLULAR UPTAKE PEPTIDE-THERAPEUTIC PAYLOAD COMPOSITIONS AND METHODS OF USE

(71) Applicant: Nighthawk Biosciences, Inc., Morrisville, NC (US)

(72) Inventors: Karla FRIETZE, Morrisville, NC (US); Neil GOLDSTEIN, Morrisville, NC (US); Jeffrey WOLF, Morrisville, NC (US); John PRENDERGAST, Morrisville, NC (US)

(21) Appl. No.: 18/725,238

(22) PCT Filed: Jan. 3, 2023
(86) PCT No.: PCT/US2023/060038

§ 371 Date: Jun. 28, 2024

Related U.S. Application Data
(60) Provisional application No. 63/295,978, filed on Jan. 3, 2022.
(60) Provisional application No. 63/394,135, filed on Aug. 1, 2022.
(60) Provisional application No. 63/417,106, filed on Oct. 18, 2022.

(51) Int. Cl. A61K 47/68; A61P 35/00; C07K 16/30

(52) U.S. Cl. CPC A61K 47/68031; A61K 47/6851; A61P 35/00; C07K 16/30

(57) ABSTRACT

The present disclosure relates to, among others, a composition comprising an internalization peptide, also called a cellular uptake peptide, connected to a drug product, also called a therapeutic payload, by a linker.

### CROSS-REFERENCE TO RELATED APPLICATIONS

This application claims priority to and the benefit of U.S. Provisional Patent Application No. 63/295,978, filed on Jan. 3, 2022, U.S. Provisional Patent Application No. 63/394,135, filed on Aug. 1, 2022, and U.S. Provisional Patent Application No. 63/417,106, filed on Oct. 18, 2022, the entire contents of which are herein incorporated by reference herein in their entireties.

### FIELD

The disclosure is related to peptides that find use in, e.g., drug delivery.

### SEQUENCE LISTING

The instant application contains a sequence listing which has been submitted in XML format via EFS-Web. The contents of the XML copy named “SKX-004_Sequence Listing”, which was created on Jan. 2, 2023 and is 111,437 bytes in size, are hereby incorporated herein by reference in their entireties.

### BACKGROUND

Peptides have various advantages as delivery agents, including: their small size e.g. to increase tumor accretion, their more consistent 1:1 peptide:drug stoichiometry (e.g. as compared to antibody-drug conjugates (ADC)); and their ease to manufacture (e.g. as compared to ADC).

There is a need for more and improved peptides for therapeutic delivery.

### SUMMARY

Accordingly, the present disclosure relates to, in aspects, a composition comprising an internalization peptide, also called a cellular uptake peptide, connected to a drug product, also called a therapeutic payload, by a linker. In embodiments, the composition is capable of delivering the drug product/therapeutic payload to a cell and having it internalized (e.g. by macropinocytosis). In embodiments, the composition further comprises an organelle uptake peptide, e.g. a nuclear localization signal (NLS). Accordingly, in embodiments, the composition is internalized into a cell and delivered inside the cell to an organelle of interest, e.g. to allow the drug product/therapeutic payload to contact a locus of action.

In embodiments, the composition further comprises one or more cleavage sites (e.g. a matrix metalloproteinase or furin cleavage site). In embodiments, the composition further comprises one or more cleavage sites allow for site-specific removal of or more components of the present compositions when the present compositions are inside of a cell. In embodiments, the cleavage site is displaced between the internalization peptide/cellular uptake peptide and the organelle uptake peptide (which is itself linked to the drug product/therapeutic payload), thereby allowing cleavage of the internalization peptide/cellular uptake peptide from the organelle uptake peptide and drug product/therapeutic payload. In embodiments, the cleavage occurs via cellular enzymes.

In embodiments, the present compositions are as shown in FIG. 8, FIG. 11, FIG. 14, FIG. 15, or FIG. 31.

In aspects, there is provided a method of delivery of a drug product/therapeutic payload to a cell by internalization comprising contact a cell, in vitro, ex vivo, or in vivo, with a composition comprising the drug product/therapeutic payload linked to an internalization peptide/cellular uptake peptide, wherein the composition optionally comprises one or more cleavage sites which allow for site-specific removal of or more components of the present compositions when they are inside of a cell.

In aspects, there is provided a method of organelle-specific delivery of a drug product/therapeutic payload comprising contact a cell, in vitro, ex vivo, or in vivo, with a composition comprising the drug product/therapeutic payload linked to an internalization peptide/cellular uptake peptide and an organelle uptake peptide, wherein the composition optionally comprises one or more cleavage sites which allow for site-specific removal of or more components of the present compositions when they are inside of a cell.

### BRIEF DESCRIPTION OF THE DRAWINGS

FIG. 1 shows sequences of 429 (IGF-1r peptide, aka SKwx-429) (SEQ ID NO: 48) and OV-8 (isolated from Ovcar-3 cells, aka Skwx-OV-8) (SEQ ID NO: 49) and two peptide-drug conjugates. DAR=Drug-Antibody Ratio.

FIG. 2 shows internalization of OV-8 in various cell lines. Skwx-OV-8 has been shown to internalize into a number of cancer cell lines of different lineages.

FIG. 3 shows dose response curves for various cell lines with the peptide drug conjugates (“PDCs”): (1) A549: human lung carcinoma, (2) SW480: human colorectal carcinoma, (3) HCT116: human colorectal carcinoma, and (4) G292: human osteosarcoma.

FIG. 4 shows that the peptide-drug conjugates kill tumor cells.

FIG. 5 shows the effects of PDCs on G292 cells.

FIG. 6 shows that MAE and PDCs disrupt microtubules.

FIG. 7 shows a 429 peptide-gemcitabine construct.

FIG. 8 shows a non-limiting schematic of various conjugated peptides of the disclosure. The internalization peptides can be, inter alia, Skwx-429 and Skwx-Ov-8 (FIG. 1). The optional FLAG sequence may be added to determine if enzyme cleavage occurred. The MMP-9 cleavage peptide is shown but Furin may be used as well. The Nuclear Localization Signal (NLS) is the sequence shown. FITC (optional) may be added to the C-term to visualize the reaction.

FIG. 9 shows nuclear localization of the 429 construct in 3 cells lines (G292 [osteosarcoma], U87 MG [glioblastoma]and HCT116 [colorectal cancer]).

FIG. 10 shows intra-nuclear staining detected with anti-FLAG antibody. As can be seen, the construct does not appear to have been cleaved but still accumulates within the nucleus of U87 cells, i.e. the entire 429 construct can localize to the nucleus.

FIG. 11 shows immunofluorescence data demonstrating that the entire 429 construct localized to the nucleus.

FIG. 12 shows constructs with 2 HDAC inhibitors as illustrative drug payloads.

FIG. 13 shows the amino acid sequence of SmarcB1 (SEQ ID NO: 122) showing potential region which binds to myc (SEQ ID NO: 123). SmarcB1 is a tumor suppressor gene which binds to and represses the proto-oncogene myc. SmarcB1 mutations have been associated with ATRT (atypical teratoid rhabdoid tumor) which is a pediatric tumor with poor prognosis.

FIG. 14 shows a prototype conjugate for proof of concept for delivering SmarcB1-myc binding sequence to nucleus (SEQ ID NO: 111).

FIG. 15 shows a non-limiting schematic of various conjugated peptides of the disclosure.

FIGS. 16A-16B show the effects of MMAE drug conjugates in G292 cells. FIG. 16A shows an image of G292 cells treated with MMAE at 50 nM, 429-MMAE at 500 nm, and 429-MMAE at 50 nM. FIG. 16B shows a bar graph of the viability of cells from various dosages of 429-MMAE and Ovalbumin(OV)-MMAE treatments.

FIG. 17 shows confocal fluorescent microscopy images of the effects of 429-MMAE on microtubules in G292 cells.

FIG. 18A-18C show 429-MMAE affecting cell viability and apoptosis in A431 cells. FIG. 18A shows an image of A431 cells treated with MMAE at 50 nM, 429-MMAE at 500 nm, and 429-MMAE at 50 nM. FIG. 18B is a bar graph depicting A431 cell viability after various 429-MMAE and OV-MMAE treatments. FIG. 18C shows a bar graph depicting A431 cell apoptosis after various 429-MMAE and OV-MMAE treatments.

FIG. 19 shows confocal fluorescent microscopy images of 429-MMAE disrupting microtubules in A431 cells.

FIGS. 20A-20B show the dose response curves of A413, HCT-116 cells, and G292 cells treated with 429-MMAE. FIG. 20A shows the dose response curves of A431 cells and HCT-116 cells treated with 429-MMAE. FIG. 20B shows the dose response curves of A431 cells and G292 cells treated with 429-MMAE.

FIGS. 21A-21C show 429-MMAE induces apoptosis in CHLA06 cells. FIG. 21A is a bar graph showing the results of a caspase assay after treating CHLA06 cells with various dosages of MMAE and 429-MMAE at 24 hours. FIG. 21B is a bar graph showing the results of a caspase assay after treating CHLA06 cells with various dosages of MMAE and 429-MMAE at 48 hours. FIG. 21C shows confocal fluorescent microscopy images of 429-MMAE induces apoptosis in CHLA06 cells

FIGS. 22A-22B show 429-MMAE-1 inhibits CHLA06 cells. FIG. 22A is a bar graph comparing CHLA06 cell viability results after various 429-MMAE-1 treatments. FIG. 22B shows confocal fluorescent microscopy images of CHLA06 cells treated with various dosages of 429-MMAE-1.

FIGS. 23A-23B show that 429-P (429-NLS-Panobinostat) inhibits CHLA06 cells. FIG. 23A shows confocal fluorescent microscopy images of CHLA06 cells treated with various dosages of 429-P. FIG. 23B is a bar graph showing the viability of CHLA06 cells treated with various dosages of Panobinostat and 429-P.

FIGS. 24A-24B show the dose response curves of A413, and CHLA06 cells treated with 429-P. FIG. 24A shows the dose response curves of A431 cells treated with 429-P and Panobinostat. FIG. 24B shows the dose response curves of CHLA06 cells treated with 429-P and Panobinostat.

FIG. 25 shows confocal microscopy images of 429-SMARCB1 internalized in CHLA-06 cells.

FIG. 26 shows confocal microscopy images of 429-SMARCB1 co-localized with histones in CHLA-06 cells.

FIGS. 27A-27C show 429-SMARCB1 inhibits Myc transcription. FIG. 27A shows a bar graph comparing HCT116 Myc-reporter cell viability after various dosages of Panobinostat. FIG. 27B shows a bar graph comparing HCT116 Myc-reporter cell viability after various dosages of 429-SMARCB1. FIG. 27C shows a line graph showing HCT116 Myc-reporter cell viability after various dosages of 429-SMARCB1.

FIG. 28A-28B show 429-SMARCB1 affects Myc transcription and viability. FIG. 28A shows a bar graph comparing HCT116 Myc-reporter cell transcription of various treatments. FIG. 28B shows a bar graph comparing HCT116 Myc-reporter cell viability after control and 429-SMARCB1 treatment.

FIG. 29 shows an PAGE gel comparing expression of SMARCB1 in atypical teratoid/rhabdoid tumor cell lines.

FIG. 30A-30B show SMARCB1 and tazemetostat reduces cell viability and reduces cell viability and increases caspase activity in CHLA06 cells. FIG. 30A is a bar graph showing the results of a cell viability assay after treating CHLA06 cells with various treatments. FIG. 30B is a bar graph showing the results of a caspase assay after treating CHLA06 cells with various treatments.

FIG. 31 shows a 429-SMARCB1 mimic conjugate (SEQ ID NO: 111) for delivering SmarcB1-myc binding sequence to nucleus.

FIG. 32A-32B show 429-SMARCB1 mimic conjugate and tazemetostat significantly increase caspase activity in CHLA06 cells. FIG. 32A shows a graph comparing various 429-SMARCB1 mimic conjugate and tazemetostat treatments in CHLA06 cells. FIG. 32B shows a line graph showing various 429-SMARCB1 mimic conjugate and tazemetostat treatments in CHLA06 cells.

FIG. 33A-33B show 429-SMARCB1 mimic conjugate and tazemetostat significantly increase caspase activity in CHLA02 cells. FIG. 33A shows a graph comparing various 429-SMARCB1 mimic conjugate and tazemetostat treatments in CHLA02 cells. FIG. 33B shows a line graph showing various 429-SMARCB1 mimic conjugate and tazemetostat treatments in CHLA02 cells.

FIG. 34A-34B show 429-SMARCB1 mimic conjugate affects viability in CHLA06 cells. FIG. 34A is a bar graph comparing viability of CHLA06 cells treated with various dosages of 429-SMARCB1 mimic conjugate. FIG. 34B is a confocal microscopy image of CHLA06 cells treated with 429-SMARCB1 mimic conjugate.

FIG. 35A-35B show 429-SMARCB1 mimic conjugates inhibits Myc transcription. FIG. 35A shows a bar graph comparing the viability of HCT116 Myc-reporter cells after treatment with various dosages of 429-SMARCB1 mimic conjugates. FIG. 35B shows a bar graph comparing the level of Myc transcription after treatment with various dosages of 429-SMARCB1 mimic conjugates.

FIG. 36A-36B shows the effects of 429 ScHv-SMARCB1 antibody and OV SMARCB1 antibody. FIG. 36A shows a confocal microscopy image of cells treated with 429 antibodies. FIG. 36B shows a bar graph comparing cell viability after treatment with various 429 constructs.

FIG. 37A-37C show the effects of 429-MMAE in nude mice. FIG. 37A shows a line graph comparing the effects of various dosages of 429-MMAE on tumor volume. FIG. 37B shows a bar graph comparing the effects of various dosages of 429-MMAE on tumor volume. FIG. 37C shows a bar graph comparing the effects of various dosages of 429-MMAE on tumor weight.

FIG. 38A-38E show a comparison of various small molecule inhibitors of IGF-1R and 429-MMAE on various cell lines. FIG. 38A shows a line graph comparing dose response of IGF-1R inhibitors and 429-MMAE on CHLA06 cells. FIG. 38B shows a line graph comparing IC50 of IGF-1R inhibitors and 429-MMAE on G292 cells. FIG. 38C shows a line graph comparing IC50 of IGF-1R inhibitors and 429-MMAE on HCT116 cells. FIG. 38D shows a line graph comparing IC50 of MMAE and 429-MMAE on WS1 cells. FIG. 38E shows a line graph comparing IC50 of MMAE and 429-MMAE on CCD841 cells.

FIG. 39A-39B show CHLA06 apoptosis with IGFR inhibitors at 48 hours and 72 hours. FIG. 39A shows a line graph comparing the IC50 of 429-MMAE and small molecule inhibitors in the induction of apoptosis in CHLA06 cells at 48 hours. FIG. 39B shows a line graph comparing the IC50 of 429-MMAE and small molecule inhibitors in the induction of apoptosis in CHLA06 cells at 72 hours.

FIG. 40 shows a line graph comparing dose response curve of 429-MMAE with and without IGF.

FIG. 41A-41C show a comparison of 429-MMAE (Drug Peptide Ratio=1) and 429-MMAE (Drug Peptide Ratio=2) in various cell lines. FIG. 41A shows aline graph comparing CHLA06 apoptosis at 48 hours after treatment of 429-MMAE (Drug Peptide Ratio=1) and 429-MMAE (Drug Peptide Ratio=2). FIG. 41B shows a line graph comparing CHLA06 dose response at day 5 after treatment of 429-MMAE (Drug Peptide Ratio=1) and 429-MMAE (Drug Peptide Ratio=2). FIG. 41C shows a line graph comparing CHLA04 dose response at day 5 after treatment of 429-MMAE (Drug Peptide Ratio=1) and 429-MMAE (Drug Peptide Ratio=2).

FIG. 42A-42D show a comparison of 429-MMAE and 429-SM in various cell lines. FIG. 42A shows a line graph comparing CHLA06 apoptosis at 48 hours after treatment of 429-MMAE and 429-SM. FIG. 42B shows a line graph comparing HCT myc at 48 hours after treatment of 429-MMAE and 429-SM. FIG. 42C shows a line graph comparing A431 dose response after treatment of 429-MMAE and 429-SM. FIG. 42D shows aline graph comparing HCT116 dose response after treatment of 429-MMAE and 429-SM.

### DETAILED DESCRIPTION

In aspects, there is provided a composition comprising an internalization peptide, also called a cellular uptake peptide connected to a drug product, also called a therapeutic payload, by a linker. Accordingly, in embodiments, the composition is capable of delivering the drug product/therapeutic payload to a cell and having it internalized (e.g. by macropinocytosis). In embodiments, the composition further comprises an organelle uptake peptide, e.g. a nuclear localization signal (NLS). Accordingly, in embodiments, the composition is internalized into a cell and delivered inside the cell to an organelle of interest, e.g. to allow the drug product/therapeutic payload to contact a locus of action.

In embodiments, the composition further comprises one or more cleavage sites (e.g. a matrix metalloproteinase or furin cleavage site). In embodiments, the composition further comprises one or more cleavage sites allow for site-specific removal of or more components of the present compositions when they are inside of a cell. In embodiments, the cleavage site is displaced between the internalization peptide/cellular uptake peptide and the organelle uptake peptide (which is itself linked to the drug product/therapeutic payload), thereby allowing cleavage of the internalization peptide/cellular uptake peptide from the organelle uptake peptide and drug product/therapeutic payload. In embodiments, the cleavage occurs via cellular enzymes.

The present compositions are alternatively referred to as “peptide-drug conjugates” or “PDCs” herein.

In aspects, there is provided a composition according to the formula:

J-Y-Z, wherein:
J comprises a cellular uptake peptide and optionally, a cell surface receptor targeting peptide and/or an organelle uptake peptide and/or a protein detection peptide
when there is a cell surface receptor targeting peptide and/or an organelle uptake peptide and/or protein detection peptide, there is optionally a cleavage site between each of these peptides and also between these peptides and the cellular uptake peptide;
the cellular uptake peptide:
   is capable of binding to a marker which is internalized by a cell, optionally expressed on a cancer cell, optionally selected from OVCAR, MIA and A549, A431, G292, HCT116, CHLA02, CHLA04, CHLA05, CHLA06, or
   the cellular uptake peptide is capable of binding to an insulin-like growth factor type 1 receptor (IGF-1R); or
   has an amino acid sequence selected from:

(SEQ ID NO: 43)
YQWVSWWHRGLLRPVLRGSLKPWYIFVRIHISWGQVHVG,
(SEQ ID NO: 43)
YQWSWWHRGLLRPVLRGSLKPWYIFVRIHISWGQVHVG,
(SEQ ID NO: 44)
TGWWFFWSYVCSVRLLAVTCSFWCTEQGEESLPRAGTHTH,
(SEQ ID NO: 45)
YTLGLGKSQRWNLFRNFFCYMPRVTVFCKSCRCVSFQTSG,
(SEQ ID NO: 46)
HYLLIRCARGFCEVRLVDAMCSITIVIDCVLFTSQRERAE,
and
(SEQ ID NO: 47)
QGSLISCWVTFVQESWDAYSLCSDLFKCQGGQVFSICEPE,
(SEQ ID NO: 48)
DFYGCLLDLSLGVPSLGWRRRCITA (″429″);
(SEQ ID NO: 49)
FSFGNSSLRKLPGWVRRRRCARGVWR (″OV-8″);
(SEQ ID NO: 48)
DFYGCLLDLSLGVPSLGWRRRCITA (″1 429-M″);
(SEQ ID NO: 49)
FSFGNSSLRKLPGVVRRRRCARGVWR (″#3 OvB8-M″);
(SEQ ID NO: 48)
DFYGCLLDLSLGVPSLGWRRRCITA (″429-F″);
and
(SEQ ID NO: 49)
FSFGNSSLRKLPGWVRRRRCARGVWR (″#4 OVB8-F″);

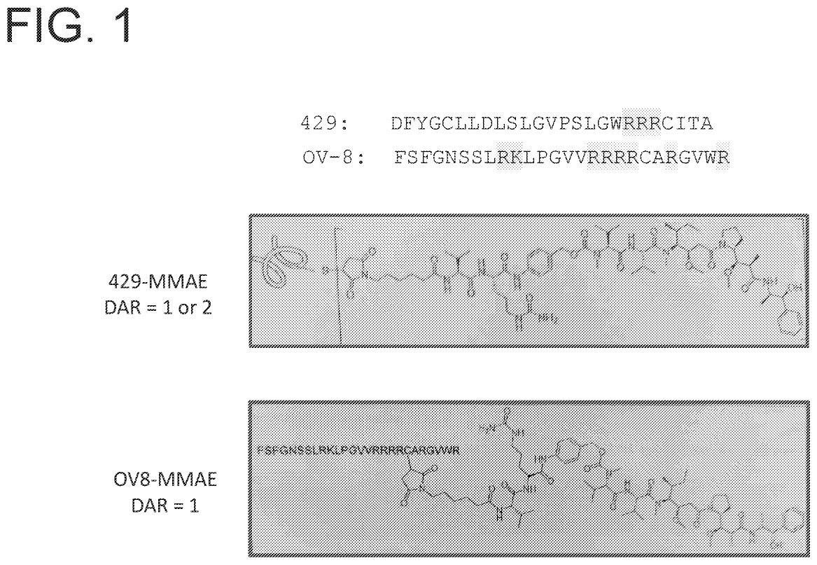

or a variant thereof, or
has an amino acid sequence selected from Table 1 or FIG. 1, or a variant thereof;
Z comprises a therapeutic payload; and
Y is a linker covalently attaching J and Z.

In embodiments, there is provided a composition according to the formula

(V)ₘ-W-(X)ₙ-Y-Z, wherein:
V comprises a cell surface receptor targeting peptide;
m is 0 or 1;
W comprises a cellular uptake peptide;
X comprises an organelle uptake peptide; and
n is 0 or 1.

In embodiments. m is 1, and V has an amino acid sequence of DFYGCLLDLSLGVPSGVRRRCITA (SEQ ID NO: 104), or a variant thereof.

In embodiments. m is 1, and V has an amino acid sequence of RPWILHLGRERSTDDQTPVHRMSLMHPLYHTRGKSYKVEG (SEQ ID NO: 105), or a variant thereof.

In embodiments, Z is or comprises 1, or 2, or 3, or 4, or 5, or 6, or 7, or 8, or 9, or 10 or more therapeutic payloads.

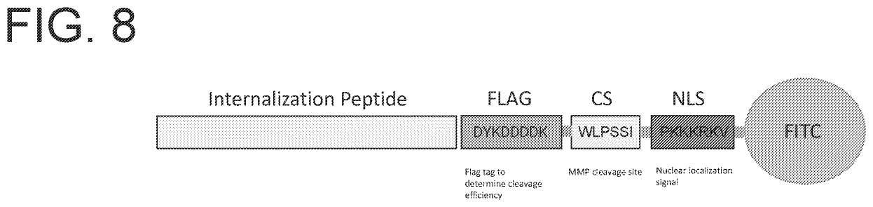

In embodiments, the present compositions are of a structure as shown in FIG. 8.

In embodiments, the present compositions are of a structure as shown in FIG. 11.

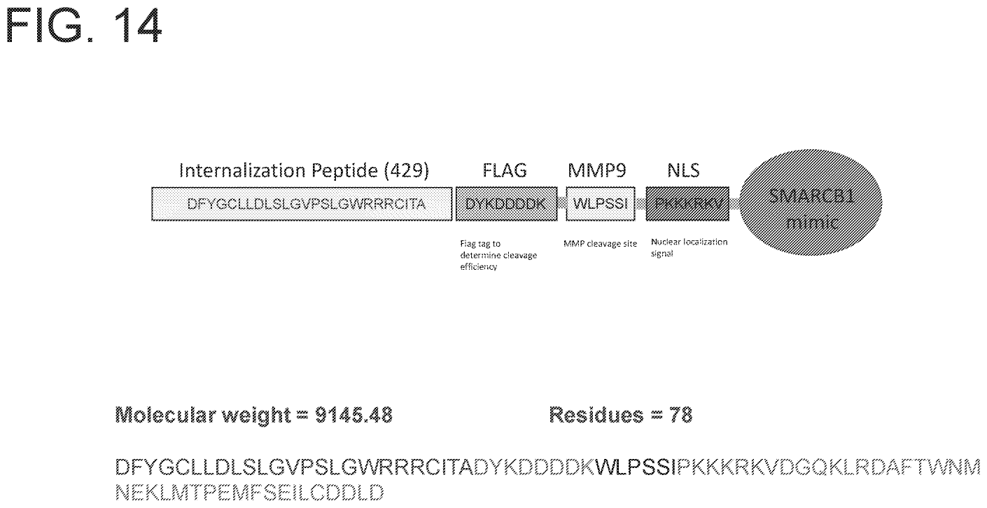

In embodiments, the present compositions are of a structure as shown in FIG. 14.

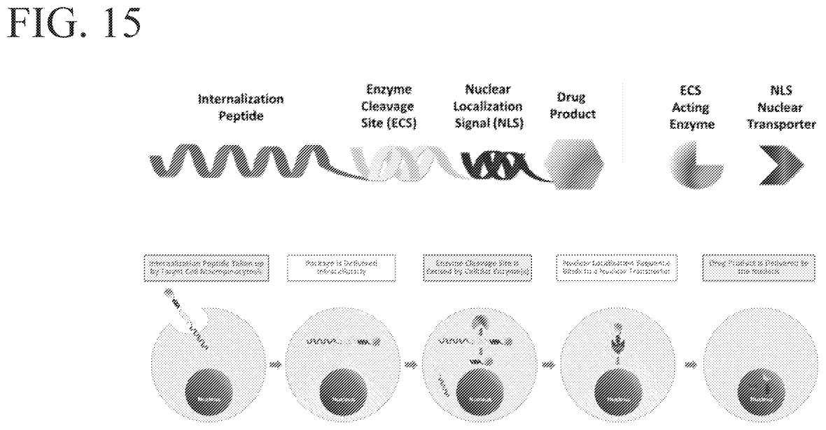

In embodiments, the present compositions are of a structure as shown in FIG. 15.

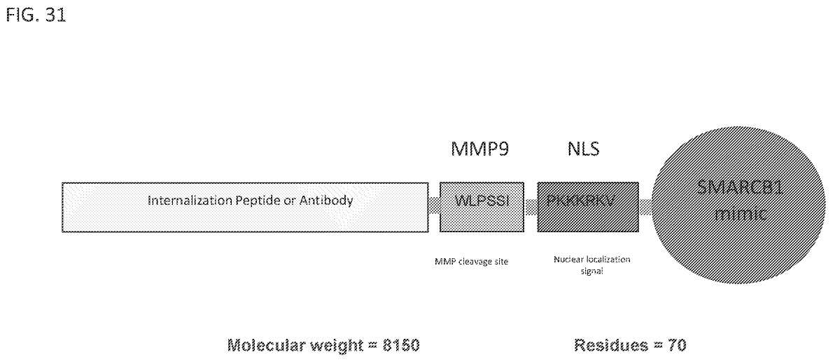

In embodiments, the present compositions are of a structure as shown in FIG. 31.

In embodiments, the present compositions comprise:

(SEQ ID NO: 113)
DFYGCLLDLSLGVPSLGWRRRCITADYKDDDDKWLPSSIPKKKRKV
(1 429-M);
(SEQ ID NO: 114)
FSFGNSSLRKLPGVVRRRRCARGVWRDYKDDDDKWLPSSIPKKKRKV
(#3 OvB8-M);
(SEQ ID NO: 115)
DFYGCLLDLSLGVPSLGWRRRCITADYKDDDDKPRRARSPKKKRKV
(429-F);
or
(SEQ ID NO: 116)
FSFGNSSLRKLPGWVRRRRCARGVWRDYKDDDDKPRRARSPKKKRKV
(#4 OVB8-F),

or variants thereof, where: DYKDDDDK (SEQ ID NO: 109)=FLAG sequence, WLPSSI (SEQ ID NO: 107)=MMP9 cleavage site, PRRARS (SEQ ID NO: 108)=furin cleavage site, and PKKKRKV (SEQ ID NO: 106)=nuclear localization signal. In embodiments, any of the FLAG sequence, MMP9 cleavage site, furin cleavage site, and nuclear localization signal may be removed from the composition.

In embodiments, the present compositions further comprise a targeting moiety. In embodiments, the targeting moiety is directed to IGFR or EGFR. In embodiments, the targeting moiety is directed to type 1 receptor (IGF-1R), epidermal growth factor receptor (EGFR), insulin receptor, GRP78/BiP, or transferrin receptor.

### Internalization Peptide/Cellular Uptake Peptide

The internalization peptide/cellular uptake peptide is sometimes referred to herein as an “internalizing peptide”, “Cell Penetrating Peptide”, or “CP3”.

In embodiments, the internalization peptide/cellular uptake peptide is capable of binding a marker, e.g. a receptor, to cause internalization by a cell. In embodiments, the internalization peptide/cellular uptake peptide is capable or being internalized by a cell by pinocytosis, e.g. micropinocytosis.

In embodiments, the internalization peptide/cellular uptake peptide is or comprises an amino acid sequence of DFYGCLLDLSLGVPSLGWRRRCITA (SEQ ID NO: 48), or a variant thereof. In embodiments, the internalization peptide/cellular uptake peptide is or comprises “429”, or a variant thereof. See FIG. 1.

In embodiments, the internalization peptide/cellular uptake peptide is or comprises an amino acid sequence of FSFGNSSLRKLPGVVRRRRCARGVWR (SEQ ID NO: 49), or a variant thereof. In embodiments, the internalization peptide/cellular uptake peptide is or comprises “OV-8”, or a variant thereof. See FIG. 1.

In embodiments, the internalization peptide/cellular uptake peptide is or comprises an amino acid sequence of one of

(SEQ ID NO: 48)
DFYGCLLDLSLGVPSLGWRRRCITA (″1 429-M″);
(SEQ ID NO: 49)
FSFGNSSLRKLPGWRRRRCARGVWR (″#3 OvB8-M″);
(SEQ ID NO: 48)
DFYGCLLDLSLGVPSLGWRRRCITA (″429-F″);
(SEQ ID NO: 49)
FSFGNSSLRKLPGWVRRRRCARGVWR (″#4 OVB8-F″);

or a variant thereof.

In embodiments, the internalization peptide/cellular uptake peptide is or comprises an amino acid sequence of one of

(SEQ ID NO: 43)
YQWSWWHRGLLRPVLRGSLKPWYIFVRIHISWGQVHVG
(″CPP 1″);
(SEQ ID NO: 44)
TGWWFFWSYVCSVRLLAVTCSFWCTEQGEESLPRAGTHTH
(″CPP 4″);
(SEQ ID NO: 45)
YTLGLGKSQRWNLFRNFFCYMPRVTVFCKSCRCVSFQTSG
(″CPP 5″);
(SEQ ID NO: 46)
HYLLIRCARGFCEVRLVDAMCSITIVIDCVLFTSQRERAE
(″CPP 6″);

or a variant thereof.

In embodiments, the internalization peptide/cellular uptake peptide is capable of binding and/or being internalized by a cancer cell. In embodiments, the cancer cell is selected from colorectal cancer cell (e.g. CaCo2), a neuroblastoma cell (e.g. SK-N-BE(2)), and/or an epidermal carcinoma cell (e.g. A431, derived from an epidermal carcinoma of the vulva taken from an 85 yr. old female). In embodiments, the cancer cell is or is derived from, OVCAR, MIA, A549, A431, G292, HCT116, CHLA02, CHLA04, CHLA05, or CHLA06 cell lines.

In embodiments, the internalization peptide/cellular uptake peptide is or comprises an amino acid sequence of Table 1, or a variant thereof.

TABLE 1
First label is cell line from which it is derived.

| Target | MA# | Sequence |
|---|---|---|
| Ovcar-3 | OVC-A1 | PRGWRRCLGWSVGPASGSGP (SEQ ID NO: 50) |
| Ovcar-3 | OVC-B8 | FSFGNSSLRKLPGVVRRRRCARGVWR (SEQ ID |
| | | NO: 49) |
| Ovcar-3 | OVC-B10 | CLGVCISGDPEQADMWGCRP (SEQ ID NO: 51) |
| Ovcar-3 | OVC-C7 | ILSRSQYLVFGYAVVARGSR (SEQ ID NO: 52) |
| Ovcar-3 | OVC-D12 | GSGLCSVCQCGGMYSRVVLGQRRWM (SEQ ID |
| | | NO: 53) |
| Ovcar-3 | OVC-E10 | GVSPIVHGDAGLLQPRNTRL (SEQ ID NO: 54) |
| Ovcar-3 | OVC-E12 | CRQRGDGGAMRRGSVCLRGW (SEQ ID NO: 55) |
| Ovcar-3 | OVC-F8 | GDGTGVQRAAVGFDLTGHSP (SEQ ID NO: 56) |
| Ovcar-3 | OVC-F12 | AVSACGRCRHCRVPGVPCWLYCVCT (SEQ ID NO: |
| | | 57) |
| Ovcar-3 | OVC-G7 | PSTMSIAGGGFCGHPRRGTI (SEQ ID NO: 58) |
| Ovcar-3 | OVC-G8 | DQCAGHDSVQRHALHGLAVM (SEQ ID NO: 59) |
| Ovcar-3 | OVC-G9 | GASPCVFRVDSTSGGPLVFPDLQST (SEQ ID NO: |
| | | 60) |
| Ovcar-3 | OVC-H8 | RREDSLTRVRQALREALGAG (SEQ ID NO: 61) |
| Ovcar-3 | OVC-H12 | LDVGVAQHRGGTVIRVAMRA (SEQ ID NO: 62) |
| MIA | MIA-A1 | GIRRGGLGRQRGWGRRRKPI (SEQ ID NO: 63) |
| MIA | MIA-A3 | RAPGGFTRTNSVRRATGSM (SEQ ID NO: 64) |
| MIA | MIA-A8 | YGKVGGWRLATVGRRTESRI (SEQ ID NO: 65) |
| MIA | MIA-A9 | KQVGLINGSACSCDMYQRVV (SEQ ID NO: 66) |
| MIA | MIA-B2 | VNSVKGVHRRAFSEQPLGSG (SEQ ID NO: 67) |
| MIA | MIA-B3 | DAGRAPALGWFFFGRRRFGE (SEQ ID NO: 68) |
| MIA | MIA-B4 | GVPGCGSHGADWGREIGATSTGMAL (SEQ ID NO: |
| | | 69) |
| MIA | MIA-B8 | GVCRWMRPHFRMPRRWRLGR (SEQ ID NO: 70) |
| MIA | MIA-C10 | RSSGMCIIARRHVLSCHSLS (SEQ ID NO: 71) |
| MIA | MIA-C11 | SGVGTCPTRPWVPGVCRQVS (SEQ ID NO: 72) |
| MIA | MIA-C8 | RVIVSGLSGRVRMGVKRRRI (SEQ ID NO: 73) |
| MIA | MIA-C9 | FMRHARPTRGVVRRIGHGV (SEQ ID NO: 74) |
| MIA | MIA-D10 | QLYACLRLVWGRQASAGWRVTMTG (SEQ ID NO: |
| | | 75) |
| MIA | MIA-D2 | LERIVGQWYSEGLSGDWANL (SEQ ID NO: 76) |
| MIA | MIA-E11 | GASRCFSLCSGSEGAESRRQATTV (SEQ ID NO: |
| | | 77) |
| MIA | MIA-E8 | HLGCSMPINVFGVCIWKCA (SEQ ID NO: 78) |
| MIA | MIA-F11 | RRLAVRLRVMLRRGPVCIRV (SEQ ID NO: 79) |
| MIA | MIA-F2 | LVDACIGSCQPEGSPPARPPFQLA (SEQ ID NO: |
| | | 80) |
| MIA | MIA-G2 | RCVGCCKYLIAQAYRFTVAGGAAQYA (SEQ ID NO: |
| | | 81) |
| MIA | MIA-G8 | AGVHCGLTNVHGVGVRSYRRLYA (SEQ ID NO: 82) |
| MIA | MIA-H5 | VNGWGLGRRGMGIDYRWAVH (SEQ ID NO: 83) |
| MIA | MIA-H8 | QSARCAVTMGLDALNLRRGISPVLD (SEQ ID NO: |
| | | 84) |
| A549 | A549-A2 | YPWQQVLVPPPGNVHAFATI (SEQ ID NO: 85) |
| A549 | A549-A3 | TAVAGVGVSAGQLQGGTAAG (SEQ ID NO: 86) |
| A549 | A549-A4 | FFGLSFPASVAVSDSGVQMR (SEQ ID NO: 87) |
| A549 | A549-B1 | SSRAVSWMVQLRVQRGSGLV (SEQ ID NO: 88) |
| A549 | A549-B3 | SRLSLVQSCSVSWGHQTVGT (SEQ ID NO: 89) |
| A549 | A549-B4 | VARWCQVNWAYGRRLAPPVG (SEQ ID NO: 90) |
| A549 | A549-C1 | FLLCSLPLTQPPYRVIGAYA (SEQ ID NO: 91) |
| A549 | A549-C3 | GGPSGIGWQHAWWVFGRQGL (SEQ ID NO: 92) |
| A549 | A549-D2 | LQRQIAGPRSRQRGRGSRYR (SEQ ID NO: 93) |
| A549 | A549-E3 | FEDMRLGWRNGQPPGGVAIW (SEQ ID NO: 94) |
| A549 | A549-F1 | DSWQGARVQPGVGAHTAQQR (SEQ ID NO: 95) |
| A549 | A549-F2 | GIKTVRGYGVWRWHGGSPRM (SEQ ID NO: 96) |
| A549 | A549-F3 | IRVWCQWLRTKLAAGQVNCA (SEQ ID NO: 97) |
| A549 | A549-F4 | SYGVGGRHSGGQGEALVYSV (SEQ ID NO: 98) |
| A549 | A549-G1 | RAQNCGNWAAVQRINVSSMA (SEQ ID NO: 99) |
| A549 | A549-G2 | GRAPEQTPCIGWRGGLIKDL (SEQ ID NO: 100) |
| A549 | A549-G3 | SGSRNFAAARWSHRGFCVPW (SEQ ID NO: 101) |
| A549 | A549-H1 | IAFSRRGRGWWCQMPGGSRV (SEQ ID NO: 102) |
| A549 | A549-H3 | GCCSAGLPSSLLSTPQSRSR (SEQ ID NO: 103) |

In embodiments, the internalization peptide/cellular uptake peptide is capable of binding to Ovcar-3 cells. In embodiments, the internalization peptide/cellular uptake peptide is or comprises an amino acid sequence of one of

(SEQ ID NO: 50)
PRGWRRCLGWSVGPASGSGP,
(SEQ ID NO: 49)
FSFGNSSLRKLPGWVRRRRCARGVWR,
(SEQ ID NO: 51)
CLGVCISGDPEQADMWGCRP,
(SEQ ID NO: 52)
ILSRSQYLVFGYAWVARGSR,
(SEQ ID NO: 53)
GSGLCSVCQCGGMYSRVVLGQRRWM,
(SEQ ID NO: 54)
GVSPIVHGDAGLLQPRNTRL,
(SEQ ID NO: 55)
CRQRGDGGAMRRGSVCLRGW,
(SEQ ID NO: 56)
GDGTGVQRAAVGFDLTGHSP,
(SEQ ID NO: 57)
AVSACGRCRHCRVPGVPCWLYCVCT,
(SEQ ID NO: 58)
PSTMSIAGGGFCGHPRRGTI,
(SEQ ID NO: 59)
DQCAGHDSVQRHALHGLAVM,
(SEQ ID NO: 60)
GASPCVFRVDSTSGGPLVFPDLQST,
(SEQ ID NO: 61)
RREDSLTRVRQALREALGAG,
and
(SEQ ID NO: 62)
LDVGVAQHRGGTVIRVAMRA,

or a variant thereof.

In embodiments, the internalization peptide/cellular uptake peptide is or comprises an amino acid sequence of FSFGNSSLRKLPGWRRRRCARGVWR (SEQ ID NO: 49), or a variant thereof.

In embodiments, the internalization peptide/cellular uptake peptide is capable of binding to MIA cells. In embodiments, the internalization peptide/cellular uptake peptide is or comprises an amino acid sequence of one of:

(SEQ ID NO: 63)
GIRRGGLGRQRGWGRRRKPI,
(SEQ ID NO: 64)
RAPGGFTRTNSVRRATGSM,
(SEQ ID NO: 65)
YGKVGGWRLATVGRRTESRI,
(SEQ ID NO: 66)
KQVGLINGSACSCDMYQRVV,
(SEQ ID NO: 67)
VNSVKGVHRRAFSEQPLGSG,
(SEQ ID NO: 68)
DAGRAPALGWFFFGRRRFGE,
(SEQ ID NO: 69)
GVPGCGSHGADWGREIGATSTGMAL,
(SEQ ID NO: 70)
GVCRWMRPHFRMPRRWRLGR,
(SEQ ID NO: 71)
RSSGMCIIARRHVLSCHSLS,
(SEQ ID NO: 72)
SGVGTCPTRPWVPGVCRQVS,
(SEQ ID NO: 73)
RVIVSGLSGRVRMGVKRRRI,
(SEQ ID NO: 74)
FMRHARPTRGVVRRIGHGV,
(SEQ ID NO: 75)
QLYACLRLVWGRQASAGWRVTMTG,
(SEQ ID NO: 76)
LERIVGQWYSEGLSGDWANL,
(SEQ ID NO: 77)
GASRCFSLCSGSEGAESRRQATTV,
(SEQ ID NO: 78)
HLGCSMPINVFGVCIWKCA,
(SEQ ID NO: 79)
RRLAVRLRVMLRRGPVCIRV,
(SEQ ID NO: 80)
LVDACIGSCQPEGSPPARPPFQLA,
(SEQ ID NO: 81)
RCVGCCKYLIAQAYRFTVAGGAAQYA,
(SEQ ID NO: 82)
AGVHCGLTNVHGVGVRSYRRLYA,
(SEQ ID NO: 83)
VNGWGLGRRGMGIDYRWAVH,
and
(SEQ ID NO: 84)
QSARCAVTMGLDALNLRRGISPVLD,

or a variant thereof.

In embodiments, the internalization peptide/cellular uptake peptide is capable of binding to A549 cells. In embodiments, the internalization peptide/cellular uptake peptide is or comprises an amino acid sequence of one of:

(SEQ ID NO: 85)
YPWQQVLVPPPGNVHAFATI,
(SEQ ID NO: 86)
TAVAGVGVSAGQLQGGTAAG,
(SEQ ID NO: 87)
FFGLSFPASVAVSDSGVQMR,
(SEQ ID NO: 88)
SSRAVSWMVQLRVQRGSGLV,
(SEQ ID NO: 89)
SRLSLVQSCSVSWGHQTVGT,
(SEQ ID NO: 90)
VARWCQVNWAYGRRLAPPVG,
(SEQ ID NO: 91)
FLLCSLPLTQPPYRVIGAYA,
(SEQ ID NO: 92)
GGPSGIGWQHAWWVFGRQGL,
(SEQ ID NO: 93)
LQRQIAGPRSRQRGRGSRYR,
(SEQ ID NO: 94)
FEDMRLGWRNGQPPGGVAIW,
(SEQ ID NO: 95)
DSWQGARVQPGVGAHTAQQR,
(SEQ ID NO: 96)
GIKTVRGYGVWRWHGGSPRM,
(SEQ ID NO: 97)
IRVWCQWLRTKLAAGQVNCA,
(SEQ ID NO: 98)
SYGVGGRHSGGQGEALVYSV,
(SEQ ID NO: 99)
RAQNCGNWAAVQRINVSSMA,
(SEQ ID NO: 100)
GRAPEQTPCIGWRGGLIKDL,
(SEQ ID NO: 101)
SGSRNFAAARWSHRGFCVPW,
(SEQ ID NO: 102)
IAFSRRGRGWWCQMPGGSRV,
and
(SEQ ID NO: 103)
GCCSAGLPSSLLSTPQSRSR,

or a variant thereof.

In embodiments, the internalization peptide/cellular uptake peptide lacks any tags (e.g. FLAG tags, His₆ tags, and the like).

### Drug Product/Therapeutic Payload

In embodiments, the therapeutic payload is a small molecule drug, biological, or radionuclide.

In embodiments, the therapeutic payload is a biological, which is selected from a toxin, an antibody, or an oligonucleotide (e.g., without limitation, antisense inhibitors).

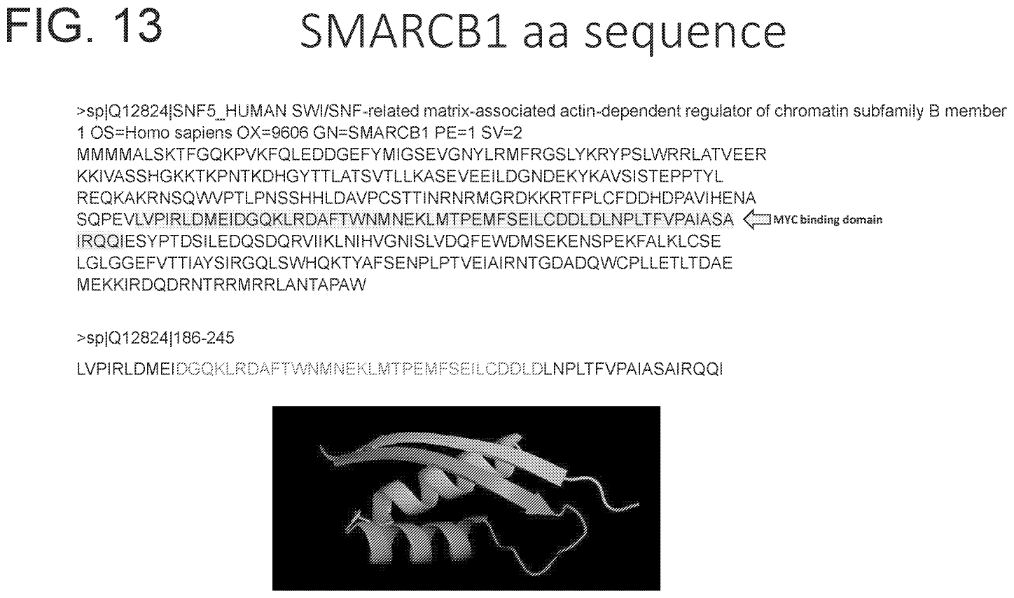

In embodiments, the therapeutic payload is a SmarcB1 mimic. In embodiments, the therapeutic payload has an amino acid sequence of FIG. 13 (SEQ ID NO: 122 or 123) or FIG. 14 (SEQ ID NO: 111). In embodiments, the composition with the SmarcB1 mimic is that of FIG. 14 (SEQ ID NO: 111).

In embodiments the SmarcB1 mimic is or comprises an amino acid sequence of

(SEQ ID NO: 122)
MMMMALSKTFGQKPVKFQLEDDGEFYMIGSEVGNYLRMFRGSLYKRYPS
LWRRLATVEERKKIVASSHGKKTKPNTKDHGYTTLATSVTLLKASEVEE
ILDGNDEKYKAVSISTEPPTYLREQKAKRNSQWVPTLPNSSHHLDAVPC
STTINRNRMGRDKKRTFPLCFDDHDPAVIHENASQPEVLVPIRLDMEID
GQKLRDAFTWNMNEKLMTPEMFSEILCDDLDLNPLTFVPAIASAIRQQI
ESYPTDSILEDQSDQRVIIKLNIHVGNISLVDQFEWDMSEKENSPEKFA
LKLCSELGLGGEFVTTIAYSIRGQLSWHQKTYAFSENPLPTVEIAIRNT
GDADQWCPLLETLTDAEMEKKIRDQDRNTRRMRRLANTAPAW

In embodiments, the SmarcB1 mimic is or comprises an amino acid sequence of

(SEQ ID NO: 123)
LVPIRLDMEIDGQKLRDAFTWNMNEKLMTPEMFSEILCDDLDLNPLTFV
PAIASAIRQQI.

In embodiments, the therapeutic payload is monomethyl auristatin E (MMAE), e.g., as shown in FIG. 1.

In embodiments, the therapeutic payload is an anticancer agent. In embodiments, the anticancer agent is a chemotherapeutic agent.

In embodiments, the chemotherapeutic agent is selected from alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above.

In embodiments, the chemotherapeutic agent is a histone deacetylase (HDAC) inhibitor (HDI). In embodiments, the HDI is selected from vorinostat, panobinostat, tazemetostat, mocetinostat, romidepsin, trichostatin A and trapoxin A.

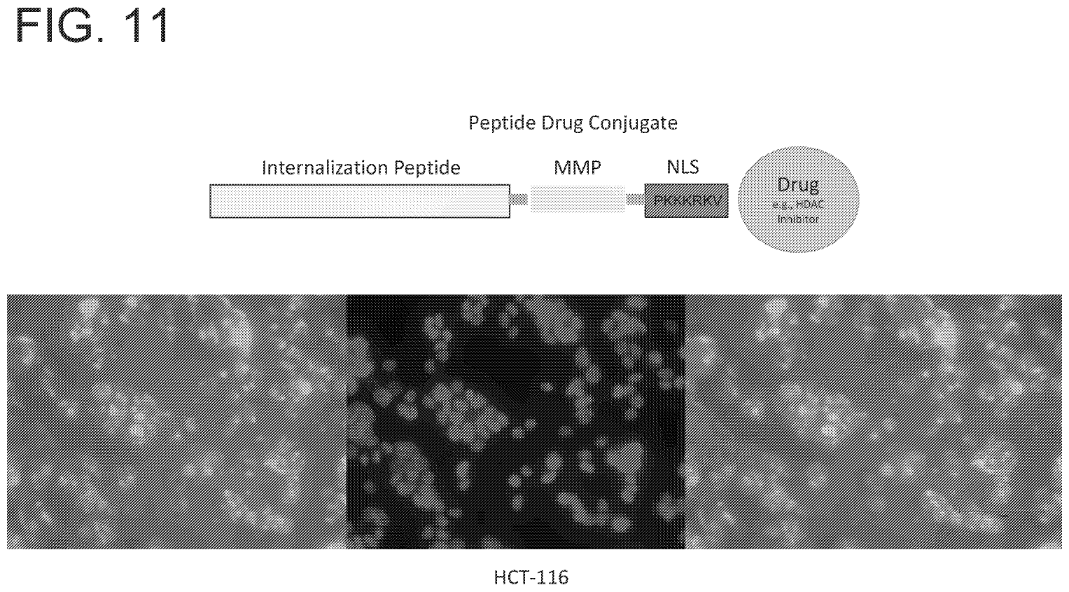

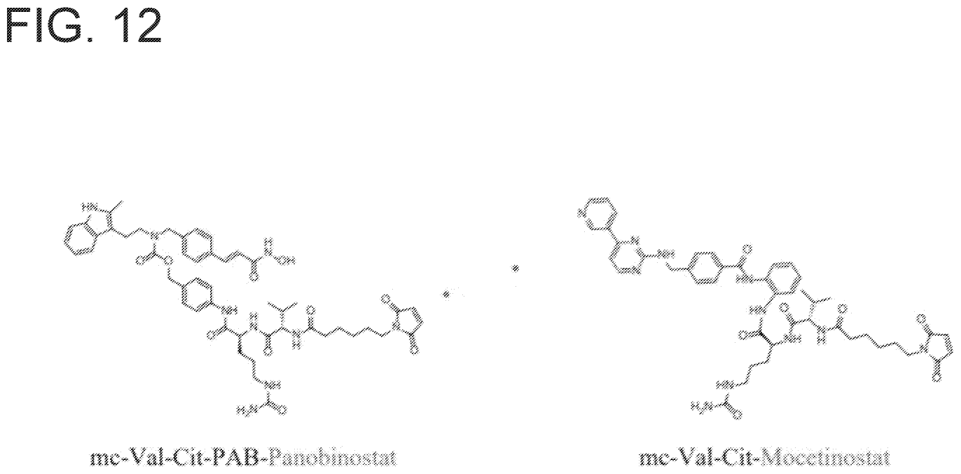

In embodiments, the therapeutic payload as shown in FIG. 12.

In embodiments, the anticancer agent is monomethyl auristatin E (MMAE).

In embodiments, the anticancer agent is an antisense oligonucleotide that binds to kras, gemcitabine, or cisplatin.

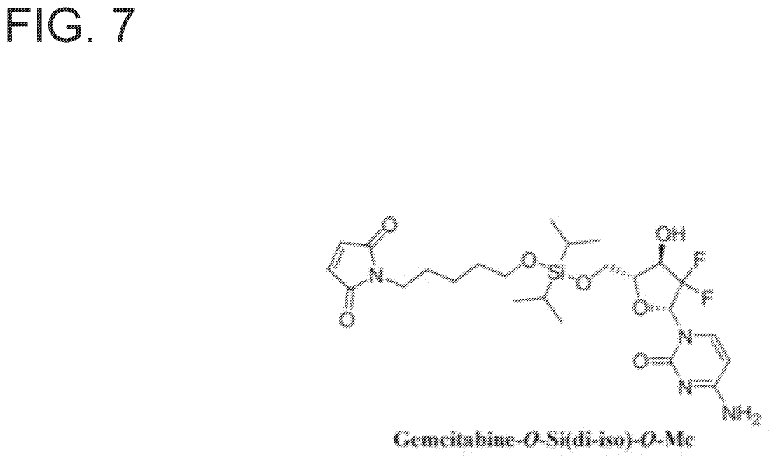

In embodiments, the therapeutic payload is gemcitabine, e.g., as shown in FIG. 7.

In embodiments, the therapeutic payload is a HDAC inhibitor.

In embodiments, the therapeutic payload is panobinostat.

In embodiments, the therapeutic payload is tazemetostat.

In embodiments, the drug peptide ratio is the ratio between the therapeutic payload and internalization peptide/cellular uptake peptide. In embodiments, the drug peptide ratio is 1, or 2, or 3, or 4, or 5, or 6, or 7, or 8, or 9, or 10 or more. In embodiments, the 429-MMAE drug peptide ratio is 1, or 2, or 3, or 4, or 5, or 6, or 7, or 8, or 9, or 10 or more.

### Organelle Uptake Peptide

In embodiments, the present compositions comprise an organelle uptake peptide which allows for uptake or internalization of the composition by a cellular organelle.

In embodiments, the organelle uptake peptide is a target peptide or signal peptide.

In embodiments, the organelle uptake peptide is a nuclear localization signal is a nuclear localization signal, optionally a classical nuclear localization signal. In embodiments, the nuclear localization signal is a monopartite classical nuclear localization signal. In embodiments, the nuclear localization signal has an amino acid sequence of PKKKRKV (SEQ ID NO: 106) or a variant thereof. In embodiments, the nuclear localization signal is a bipartite classical nuclear localization signal. In embodiments, the nuclear localization signal is a non-classical nuclear localization signal.

In embodiments, the organelle uptake peptide is an endoplasmic reticulum localization signal.

### Cleavage Sites

In embodiments, the composition further comprises one or more cleavage sites (e.g., a matrix metalloproteinase or furin cleavage site). In embodiments, V or W or X further comprise a cleavage site.

In embodiments, the composition further comprises one or more cleavage sites allow for site-specific removal of or more components of the present compositions when they are inside of a cell. In embodiments, the cleavage site is displaced between the internalization peptide/cellular uptake peptide and the organelle uptake peptide (which is itself linked to the drug product/therapeutic payload), thereby allowing cleavage of the internalization peptide/cellular uptake peptide from the organelle uptake peptide and drug product/therapeutic payload. In embodiments, the cleavage occurs via cellular enzymes.

In embodiments, the cleavage site is a matrix metalloproteinase cleavage site, e.g., one of the following:

| MMPs | Biological functions |
|---|---|
| MMP-1 | Induces cell migration and invasion and reduces |
| | proliferation |
| MMP-2 | ECM disassembly, increases cell proliferation, |
| | invasion/migration and angiogenesis |
| MMP-7 | Apoptosis, tumor invasion and metastasis |
| MMP-9 | ECM remodeling, increases cell proliferation, |
| | invasion/migration and angiogenesis |
| MMP-12 | Tumor suppressor, ECM remodeling, increases |
| | epithelial cell proliferation involved in wound healing |
| MMP-13 | Promotes tumor angiogenesis, ECM disassembly, |
| | bone mineralization and morphogenesis |
| MMP-26 | Collagen catabolic process, decreases |
| | inflammatory response and proteolysis |

In embodiments, the cleavage site is a matrix metalloproteinase 9 cleavage site. In embodiments, the cleavage site has an amino acid sequence of WLPSSI (SEQ ID NO: 107).

In embodiments, the cleavage site is a furin cleavage site. In embodiments, the cleavage site has an amino acid sequence of PRRARS (SEQ ID NO: 108).

### Targeting Moiety/Cell Surface Receptor Targeting Peptide

In embodiments, the present compositions comprise a targeting moiety/cell surface receptor targeting peptide.

In embodiments, the targeting moiety/cell surface receptor targeting peptide binds to a cancer cell surface receptor or a microvascular endothelial cell surface receptor. In embodiments, the cancer cell surface receptor is insulin-like growth factor type 1 receptor (IGF-1R), epidermal growth factor receptor (EGFR), insulin receptor, GRP78/BiP, or transferrin receptor.

In embodiments, the targeting moiety/cell surface receptor targeting peptide is IGFR or EGFR specific, e.g. RPWILHLGRERSTDDQTPVHRMSLMHPLYHTRGKSYKVEG (SEQ ID NO: 105) or a variant thereof) and is optionally linked to an MMP9 cleavage site further linked to the internalizing peptide, e.g. Ov8 or Skwx-429, and optionally further linked to the drug product/therapeutic payload.

In embodiments, the present compositions comprise:

EGFR binder(in bold)-MMP9 cleavage site(underlined)-429(no markings)35-429
(SEQ ID NO: 117)
RPWILHLGRERSTDDQTPVHRMSLMHPLYHTRGKSYKVEGWLPSSIDFYGCLLDLSLGVPSGVRRRCITA-
linker-drug product/therapeutic payload
EGFR binder-MMP9 cleavage site-Ov8 35-OVB8
(SEQ ID NO: 119)
RPWILHLGRERSTDDQTPVHRMSLMHPLYHTRGKSYKVEGWLPSSIFSFGNSSLRKLPGWVRRRRCARGV
WR-linker-drug product/therapeutic payload

In embodiments, the present compositions lack any tags (e.g., FLAG tags, His₆ tags, and the like).

### GPNMB and Drug Peptide Conjugate

In aspects, the present disclosure provides a composition comprising a GPNMB recognition domain linked to a therapeutic payload. In embodiments, the composition comprises a GPNMB recognition domain linked to 1, or 2, or 3, or 4, or 5, or 6, or 7, or 8, or 9, or 10 or more therapeutic payloads. In embodiments, the composition comprises a peptide, a small protein, or an antibody (e.g., a single-chain antibody), that binds to GPNMB. In embodiments, the composition is internalized by GPNMB expressed on the surface of a cell.

In embodiments, a single-chain antibody is a single-chain variable fragment (scFv). In embodiments, the composition comprises a single-chain antibody, small protein, or a single polypeptide that contains at least one variable binding domain (e.g., a variable domain of a mammalian heavy or light chain immunoglobulin, a camelid VHH, or a cartilaginous fish (e.g., shark) Ig-NAR domain) that is capable of specifically binding to an antigen, for example GPNMB. Non-limiting examples of single-chain antibodies include single-domain antibodies.

In embodiments, the term “single-domain antibody” refers to a polypeptide that contains one camelid VHH or at least one cartilaginous fish Ig-NAR domain that is capable of specifically binding to an antigen. Non-limiting examples of single-domain antibodies are described, for example, in U.S. Publication No. 2010/0092470.

In embodiments, the small protein ranges in size from about 2 kDa to about 4 kDa. In embodiments, the small protein ranges in size from about 2 kDa to about 3.5 kDa, from about 2 kDa to about 3 kDa, or from about 2 kDa to about 2.5 kDa.

In embodiments, the peptide or small protein is an agonist. In embodiments, the peptide or small protein is an antagonist.

In embodiments, the antibody ranges in size from about 5 kDa to about 30 kDa, from about 5 kDa to about 25 kDa, from about 5 kDa to about 20 kDa, or from about 5 kDa to about 15 kDa. In embodiments, the antibody (e.g., a single-chain antibody) is a 12 kDa fragment.

In embodiments, the peptide or small protein retains the biological function of the antibody. In embodiments, the small protein and antibody compositions are interchangeable. In embodiments, the peptide or small protein is derived from the binding region of the antibody. In embodiments, the target specific small protein is incorporated into the binding regions of the antibody (e.g., a single-chain antibody).

In embodiments, a single-chain antibody is a single-chain variable fragment (scFv). In embodiments, the composition comprises a single-chain antibody, small protein, or a single polypeptide that contains at least one variable binding domain (e.g., a variable domain of a mammalian heavy or light chain immunoglobulin, a camelid VHH, or a cartilaginous fish (e.g., shark) Ig-NAR domain) that is capable of specifically binding to an antigen. Non-limiting examples of single-chain antibodies include single-domain antibodies.

In embodiments, the composition comprises a single-chain antibody, or fragment thereof, having a heavy chain variable domain. The variable domain determines the specificity of the antibody. Each variable region comprises three hypervariable regions also known as complementarity determining regions (CDRs) flanked by four relatively conserved framework regions (FRs). The three CDRs, referred to as CDR1, CDR2, and CDR3, contribute to the antibody binding specificity. In embodiments, the single-chain antibody is a chimeric antibody. In embodiments, the single-chain antibody is a humanized antibody.

In embodiments, the present disclosure provides a composition which specifically binds to glycoprotein non-metastatic melanoma protein b (GPNMB), wherein the composition comprises, a single-chain antibody, or fragment thereof comprising CDR1, CDR2, and CDR3, wherein, CDR1 is or comprises MIHYLGWSKV (SEQ ID NO: 1), or YMGSRQNDMG (SEQ ID NO: 2), or LNRAPYFPNA (SEQ ID NO: 3), or WLSNNFWAW (SEQ ID NO: 4) or a variant thereof, wherein the variant comprises about 1, or about 2, or about 3, or about 4, or about 5 mutations, the mutations selected from substitutions or deletions, CDR2 is or comprises QIKMWWAMMH (SEQ ID NO: 5) or TSFWIKARKW (SEQ ID NO: 6), or WASWRWFRDH (SEQ ID NO: 7), or QTLKTSWWWT (SEQ ID NO: 8), or a variant thereof, wherein the variant comprises about 1, or about 2, or about 3, or about 4, or about 5 mutations, the mutations selected from substitutions or deletions, and CDR3 is or comprises LPGGNNFNLYRSFIMTLW (SEQ ID NO: 9) or WQLGSGGLHVWVSCHMYGGVYGKTSELALTWPGMWTRTHLW (SEQ ID NO: 10), or DSPRLDSIWSMSHAMGHL (SEQ ID NO: 11), or YLVRFRLDGVVWHEVVGNVNEYRTAWASIFDWLAKFQLGS (SEQ ID NO: 12), or ILNSCNPTWTRLQFKFCPALLMNGPIFAWFLWAAWVSSAM (SEQ ID NO: 13), or SDEYRFDRSVESVWQDLVIYQSHVLWWTTVHRYLAAWIQW (SEQ ID NO: 14), or VNWINMHSAKWMWWTHGV (SEQ ID NO: 15), or TPTLNYGNFNERDMDTVG (SEQ ID NO: 16), or a variant thereof, wherein the variant comprises about 1, or about 2, or about 3, or about 4, or about 5 mutations, the mutations selected from substitutions or deletions; or a peptide, the peptide selected from, LPGGNNFNLYRSFIMTLW (SEQ ID NO: 9), or a variant thereof; WQLGSGGLHVWVSCHMYGGVYGKTSELALTWPGMWTRTHLW (SEQ ID NO: 10), or a variant thereof; DSPRLDSIWSMSHAMGHL (SEQ ID NO: 11), or a variant thereof; YLVRFRLDGVVWHEVVGNVNEYRTAWASIFDWLAKFQLGS (SEQ ID NO: 12), or a variant thereof; ILNSCNPTWTRLQFKFCPALLMNGPIFAWFLWAAWVSSAM (SEQ ID NO: 13), or a variant thereof; CVLGDYHYLYTGSADHTLRESLSLLTRCRFRRVIYQQQLC (SEQ ID NO: 120), or a variant thereof; FTHISRFGEQHPTLVNLFYPRWQYIFIFTRNALCGQYVWD (SEQ ID NO: 17), or a variant thereof; LSDFEINKRCVLGVFYYLGEGSRGHILSFFHPNVQQFRVP (SEQ ID NO: 18), or a variant thereof; GDFGNRYIQLLVIVLSNGQIFVQKLLSMRPFTINGDRLRQ (SEQ ID NO: 19), or a variant thereof; ASVTLFRLVPMQNAWVTVLPSFAEFVMARPIEERATWGGS (SEQ ID NO: 20), or a variant thereof; WQLGSGGLHVWVSCHMYGGVYGKTSELALTWPGMWTRTHL (SEQ ID NO: 21), or a variant thereof; DSTMTLLARANSHASLLNVYLNINQKVEDVFFMLFLGSPP (SEQ ID NO: 22), or a variant thereof; LATWIQGSKIWSNETYSISIQVSWLSLFVMGQNLYYLLSV (SEQ ID NO: 23), or a variant thereof; SDEYRFDRSVESVWQDLVIYQSHVLWWTTVHRYLAAWIQW (SEQ ID NO: 14), or a variant thereof; VNWINMHSAKWMWWTHGV (SEQ ID NO: 15), or a variant thereof; TPTLNYGNFNERDMDTVG (SEQ ID NO: 16), or a variant thereof; QKVLYTAVPFKHGSYIFCDRIGFIFNYICEFSRSWSTWSM (SEQ ID NO: 24), or a variant thereof; VGNSIQYVKAVQTQYWAAITSWIPITRFTTRVMFLLPQVM (SEQ ID NO: 25), or a variant thereof; QVTARCLQIIVLYKLGNNGVTSLFHVYAMSSVPKFSVKWS (SEQ ID NO: 26), or a variant thereof; GQVCSPFVTSALKLFGADFGWWSLVLMKAVSWVLYITFNA (SEQ ID NO: 27), or a variant thereof; TLDHVRQAYAALSYSTVRCTFVSQMTIFYFGRVMFKALCN (SEQ ID NO: 28), or a variant thereof; AYHLQLEQLRCVSQNWTSFLDFALTKALALRSVLLGVWM (SEQ ID NO: 29), or a variant thereof; QCFQDDLCVLPVASLLDHTRSMSHLFEGWLPSRSFGFRNG (SEQ ID NO: 30), or a variant thereof; STAWIWQDWFRTFRMAVVSSARDEAVDRVCLAWPARYM (SEQ ID NO: 31), or a variant thereof; LSAADMYKHKIVGEAGEPIFYMVRNWLQKQWPMNFILMSL (SEQ ID NO: 32), or a variant thereof; FLWQWSADSMGAVDACSHKILGLFYHSQNIFARPFLRSYG (SEQ ID NO: 33), or a variant thereof; NIFIPPVGYYLTKQCGTNNFWFYQVLCRRWGADSILFWER (SEQ ID NO: 34), or a variant thereof, wherein the variant comprises about 1, or about 2, or about 3, or about 4, or about 5 mutations, the mutations selected from substitutions or deletions.

In embodiments, the GPNMB single chain antibody comprises CDRs, wherein CDR1 is or comprises MIHYLGWSKV (SEQ ID NO: 1), CDR2 is or comprises QIKMWWAMMH (SEQ ID NO: 5), and CDR3 is or comprises LPGGNNFNLYRSFIMTLW (SEQ ID NO: 9) or a variant thereof, wherein the variant comprises about 1, or about 2, or about 3, or about 4, or about 5 mutations, the mutations selected from substitutions or deletions.

In embodiments, the GPNMB single chain antibody comprises CDRs, wherein CDR1 is or comprises MIHYLGWSKV (SEQ ID NO: 1), CDR2 is or comprises QIKMWWAMMH (SEQ ID NO: 5), and CDR3 is or comprises WQLGSGGLHVWVSCHMYGGVYGKTSELALTWPGMWTRTHLW (SEQ ID NO: 10) or a variant thereof, wherein the variant comprises about 1, or about 2, or about 3, or about 4, or about 5 mutations, the mutations selected from substitutions or deletions.

In embodiments, the GPNMB single chain antibody comprises CDRs, wherein CDR1 is or comprises YMGSRQNDMG (SEQ ID NO: 2), CDR2 is or comprises TSFWIKARKW (SEQ ID NO: 6), CDR3 is or comprises DSPRLDSIWSMSHAMGHL (SEQ ID NO: 11) or a variant thereof, wherein the variant comprises about 1, or about 2, or about 3, or about 4, or about 5 mutations, the mutations selected from substitutions or deletions.

In embodiments, the GPNMB single chain antibody comprises CDRs, wherein CDR1 is or comprises YMGSRQNDMG (SEQ ID NO: 2), CDR2 is or comprises TSFWIKARKW (SEQ ID NO: 6), and CDR3 is or comprises YLVRFRLDGWWHEWGNVNEYRTAWASIFDWLAKFQLGS (SEQ ID NO: 12) or a variant thereof, wherein the variant comprises about 1, or about 2, or about 3, or about 4, or about 5 mutations, the mutations selected from substitutions or deletions.

In embodiments, the GPNMB single chain antibody comprises CDRs, wherein CDR1 is or comprises LNRAPYFPNA (SEQ ID NO: 3), CDR2 is or comprises WASWRWFRDH (SEQ ID NO: 7), and CDR3 is or comprises ILNSCNPTWTRLQFKFCPALLMNGPIFAWFLWAAWVSSAM (SEQ ID NO: 13) or a variant thereof, wherein the variant comprises about 1, or about 2, or about 3, or about 4, or about 5 mutations, the mutations selected from substitutions or deletions.

In embodiments, the GPNMB single chain antibody comprises CDRs, wherein CDR1 is or comprises WLSNNFWAW (SEQ ID NO: 4), CDR2 is or comprises QTLKTSWWWT (SEQ ID NO: 8), and CDR3 is or comprises SDEYRFDRSVESVWQDLVIYQSHVLWWTTVHRYLAAWIQW (SEQ ID NO: 14) or a variant thereof, wherein the variant comprises about 1, or about 2, or about 3, or about 4, or about 5 mutations, the mutations selected from substitutions or deletions.

In embodiments, the GPNMB single chain antibody comprises CDRs, wherein CDR1 is or comprises LNRAPYFPNA (SEQ ID NO: 3), CDR2 is or comprises WASWRWFRDH (SEQ ID NO: 7), and CDR3 is or comprises VNWINMHSAKWMWWTHGV (SEQ ID NO: 15) or a variant thereof, wherein the variant comprises about 1, or about 2, or about 3, or about 4, or about 5 mutations, the mutations selected from substitutions or deletions.

In embodiments, the GPNMB single chain antibody comprises CDRs, wherein CDR1 is or comprises WLSNNFWAW (SEQ ID NO: 4), CDR2 is or comprises QTLKTSWWWT (SEQ ID NO: 8), and CDR3 is or comprises TPTLNYGNFNERDMDTVG (SEQ ID NO: 16) or a variant thereof, wherein the variant comprises about 1, or about 2, or about 3, or about 4, or about 5 mutations, the mutations selected from substitutions or deletions.

In embodiments, the GPNMB single-chain antibody, or fragment thereof, further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

In embodiments, the variable region FW sequences are human variable region FW sequences.

In embodiments, the GPNMB single-chain antibody comprises an amino acid sequence of, MAQVQLVESGGGVVQPGRSLRLSCAASMIHYLGWSKVWFRQAPGKEREFVAQIKMWWAMMHYADSVKGRFTISR DNSKNTLYLQMNSLRAEDTAVYYCARLPGGNNFNLYRSFIMTLWWGQGTLVTVSSGPGGQ (SEQ ID NO: 35), or an amino acid sequence having at least about 90%, or at least about 95%, or at least about 97%, or at least about 98%, or at least about 99% identity thereto.

In embodiments, the GPNMB single-chain antibody comprises an amino acid sequence of, MAQVQLVESGGGVVQPGRSLRLSCAASMIHYLGWSKVWFRQAPGKEREFVAQKMWWAMMHYADSVKGRFTISR DNSKNTLYLQMNSLRAEDTAVYYCARWQLGSGGLHVWVSCHMYGGVYGKTSELALTWPGMWTRTHLWWGQGTL VTVSSGPGGQ (SEQ ID NO: 36), or an amino acid sequence having at least about 90%, or at least about 95%, or at least about 97%, or at least about 98%, or at least about 99% identity thereto.

In embodiments, the GPNMB single-chain antibody comprises an amino acid sequence of, MAQVQLVESGGGVVQPGRSLRLSCAASYMGSRQNDMGWFRQAPGKEREFVATSFWIKARKWYADSVKGRFTISR DNSKNTLYLQMNSLRAEDTAVYYCARDSPRLDSIWSMSHAMGHLWGQGTLVTVSSGPGGQ (SEQ ID NO: 37), or an amino acid sequence having at least about 90%, or at least about 95%, or at least about 97%, or at least about 98%, or at least about 99% identity thereto.

In embodiments, the GPNMB single-chain antibody comprises an amino acid sequence of, MAQVQLVESGGGVVQPGRSLRLSCAASYMGSRQNDMGWFRQAPGKEREFVATSFWIKARKWYADSVKGRFTISR DNSKNTLYLQMNSLRAEDTAVYYCARYLVRFRLDGVVWHEVVGNVNEYRTAWASIFDWLAKFQLGSWGQGTLVTV SSGPGGQ (SEQ ID NO: 38), or an amino acid sequence having at least about 90%, or at least about 95%, or at least about 97%, or at least about 98%, or at least about 99% identity thereto.

In embodiments, the GPNMB single-chain antibody comprises an amino acid sequence of, MQVQLVESGGGVVQPGRSLRLSCAASLNRAPYFPNAWFRQAPGKEREFVAWASWRWFRDHYADSVKGRFTISRD NSKNTLYLQMNSLRAEDTAVYYCARILNSCNPTWTRLQFKFCPALLMNGPIFAWFLWAAWVSSAMWGQGTLVTVSS GPGGQ (SEQ ID NO: 39), or an amino acid sequence having at least about 90%, or at least about 95%, or at least about 97%, or at least about 98%, or at least about 99% identity thereto.

In embodiments, the GPNMB single-chain antibody comprises an amino acid sequence of, MQVQLVESGGGVVQPGRSLRLSCAASSWLSNNFWAWWFRQAPGKEREFVAQTLKTSWWWTYADSVKGRFTISR DNSKNTLYLQMNSLRAEDTAVYYCARSDEYRFDRSVESVWQDLVIYQSHVLWWTTVHRYLAAWIQWWGQGTLVTV SSGPGGQ (SEQ ID NO: 40), or an amino acid sequence having at least about 90%, or at least about 95%, or at least about 97%, or at least about 98%, or at least about 99% identity thereto.

In embodiments, the GPNMB single-chain antibody comprises an amino acid sequence of, MQVQLVESGGGVVQPGRSLRLSCAASLNRAPYFPNAWFRQAPGKEREFVAWASWRWFRDHYADSVKGRFTISRD NSKNTLYLQMNSLRAEDTAVYYCARVNWINMHSAKWMWWTHGVWGQGTLVTVSSGPGGQ (SEQ ID NO: 41), or an amino acid sequence having at least about 90%, or at least about 95%, or at least about 97%, or at least about 98%, or at least about 99% identity thereto.

In embodiments, the GPNMB single-chain antibody comprises an amino acid sequence of, MQVQLVESGGGVVQPGRSLRLSCAASSWLSNNFWAWWFRQAPGKEREFVAQTLKTSWWWTYADSVKGRFTISR DNSKNTLYLQMNSLRAEDTAVYYCARTPTLNYGNFNERDMDTVGWGQGTLVTVSSGPGGQ (SEQ ID NO: 42), or an amino acid sequence having at least about 90%, or at least about 95%, or at least about 97%, or at least about 98%, or at least about 99% identity thereto.

In embodiments, the therapeutic payload is an anticancer agent.

In embodiments, the therapeutic payload is a small molecule drug, biological, or radionuclide.

In embodiments, the therapeutic payload is a biological, which is selected from a toxin, an antibody, or an oligonucleotide (e.g., without limitation, antisense inhibitors).

In embodiments, the therapeutic payload is monomethyl auristatin E (MMAE).

In embodiments, the therapeutic payload is a histone deacetylase (HDAC) inhibitor. In embodiments, the HDAC inhibitor is selected from vorinostat, panobinostat, tazemetostat, mocetinostat, romidepsin, trichostatin A and trapoxin A.

In embodiments, the therapeutic payload is gemcitabine, cisplatin, or an antisense oligonucleotide that binds to kras.

In embodiments, the therapeutic payload is a SmarcB1 mimic.

In embodiments, the therapeutic payload is an anticancer agent. In embodiments, the anticancer agent is a chemotherapeutic agent.

In embodiments, the chemotherapeutic agent is selected from alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above.

### Linkers

In embodiments, the present compositions comprise one or more linkers.

In embodiments, the linker is of the type employed in ADCs, see e.g., Su, et al. Acta Pharmaceutica Sinica B, Volume 11, Issue 12, December 2021, Pages 3889-390, the entire contents of which are hereby incorporated by reference. In embodiments, the linker is a cleavable linker. In embodiments, the linker has a chemical trigger in its structure that can be cleaved to release components of the composition. In embodiments, the linker is noncleavable.

In embodiments, the linker is a valine-citrulline (Val-Cit) linker. In embodiments, the linker is mc-Val-Cit. In embodiments, the linker is MC-Val-Cit-PAB. In embodiments, the linker is MC-Val-Cit-PAB-PNP. In embodiments, the linker is that shown in FIG. 1. In embodiments, the linker is that shown in FIG. 12. In embodiments, the linker is a triglycyl peptide linker (CX).

In embodiments, the linker is a flexible linker. In embodiments, the linker is substantially comprised of glycine and serine residues. In embodiments, the linker is (GGS)ₙ, wherein ₙ is 1, or 2, or 3, or 4, or 5. In embodiments, the linker is GGSGGSGGSG (SEQ ID NO: 118), or a variant thereof, wherein the variant comprises about 1, or about 2, or about 3, or about 4, or about 5 mutations, the mutations selected from substitutions or deletions.

In embodiments, the linker further comprises a cleavage site. In embodiments, the cleavage site is a matrix metalloproteinase cleavage site, optionally a matrix metalloproteinase 9 cleavage site, optionally having the amino acid sequence of WLPSSI (SEQ ID NO: 107) or a furin cleavage site, optionally having the amino acid sequence of PRRARS (SEQ ID NO: 108).

### Exemplary Constructs

In embodiments, the composition comprises an amino acid sequence of DFYGCLLDLSLGVPSLGWRRRCITADYKDDDDKWLPSSIPKKKRKVDGQKLRDAFTWNMNEKLMTPEMFSEILCDDL D (SEQ ID NO: 110), or an amino acid sequence having at least about 90%, or at least about 95%, or at least about 97%, or at least about 98%, or at least about 99% identity thereof.

In embodiments, the composition comprises an amino acid sequence of DFYGCLLDLSLGVPSLGWRRRCITAWLPSSIPKKKRKVDGQKLRDAFTWNMNEKLMTPEMFSEILCDDLD (SEQ ID NO: 111), or an amino acid sequence having at least about 90%, or at least about 95%, or at least about 97%, or at least about 98%, or at least about 99% identity thereof.

In embodiments, the composition comprises an amino acid sequence of DFYGCLLDLSLGVPSLGWRRRCITAWLPSSIPKKKRKVDGQKLRDAFTWNMNEKLMTPEMFSEILADDLD (SEQ ID NO: 121), or an amino acid sequence having at least about 90%, or at least about 95%, or at least about 97%, or at least about 98%, or at least about 99% identity thereof.

In embodiments, the composition comprises an amino acid sequence of QVQLVESGGGVVQPGRSLRLSCAASGGGGSGGGGSWFRQAPGKEREFVAGGGGSGGGGSYADSVKGRFTISRD NSKNTLYLQMNSLRAEDTAVYYCARDFYGCLLDLSLGVPSLGWRRRCITAWGQGLVTVSSGPGGQWLPSSIPKKKR KVDGQKLRDAFTWNMNEKLMTPEMFSEILCDDLD (SEQ ID NO: 112), or an amino acid sequence having at least about 90%, or at least about 95%, or at least about 97%, or at least about 98%, or at least about 99% identity thereof.

In embodiments, the composition is suitable for administration with one or more additional therapeutic agents. In embodiments, the additional agent is tazemetostat.

In embodiments, the composition is suitable for administration with one or more additional treatments. In embodiments, other treatments that can be used in combination with the composition as provided herein include, for example, radiation therapy, chemotherapy, hormonal therapy, and the use of angiogenesis inhibitors. Further combination partners that may be useful include checkpoint inhibitors (e.g., anti-PD1/L1, anti-CTLA-4, anti-LAG3, anti-B7-H3, anti-B7-H4, anti-TIM3, anti-TIGIT, anti-CD47, anti-TMIGD2, anti-BTLA, anti-CEACAM, or anti-GARP), other costimulatory antibodies (e.g., anti-OX40, anti-ICOS, anti-CD137, anti-GITR, or anti-CD40), cancer vaccines (e.g., virus based vaccines, peptide vaccines, whole-cell vaccines, or RNA based vaccines), and targeted agents [e.g., HERCEPTIN® (trastuzumab), TARCEVA® (erlotinib), AVASTIN® (bevacizumab), or IMBRUVICA® (ibrutinib)].

### Variants

In embodiments, a variant as used herein has or comprises about 1, or about 2, or about 3, or about 4, or about 5, or about 6, or about 7, or about 8, or about 9, or about 10 mutations. In embodiments, the mutations are selected from substitutions or deletions.

In embodiments, a variant as used herein has at least about 90%, or at least about 93%, or at least about 95%, or at least about 97%, or at least about 98% identity to a sequence described herein.

In embodiments, disclosed herein are one or more (e.g. about 1, or about 2, or about 3, or about 4, or about 5, or about 6, or about 7, or about 8, or about 9, or about 10, or about 15, or about 20, or about 30) substitutions to a sequence described herein or a sequence with at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.8, 99.9% identity to a sequence described herein (or at least about 70%, or at least about 75%, or at least about 80%, or at least about 85%, or at least about 90%, or at least about 95%, or at least about 96%, or at least about 97%, or at least about 98%, or at least about 99% identity to a sequence described herein). In embodiments, one or more amino acids of a sequence described herein is substituted with a naturally occurring amino acid, such as a hydrophilic amino acid (e.g. a polar and positively charged hydrophilic amino acid, such as arginine (R) or lysine (K); a polar and neutral of charge hydrophilic amino acid, such as asparagine (N), glutamine (Q), serine (S), threonine (T), proline (P), and cysteine (C), a polar and negatively charged hydrophilic amino acid, such as aspartate (D) or glutamate (E), or an aromatic, polar and positively charged hydrophilic amino acid, such as histidine (H)) or a hydrophobic amino acid (e.g. a hydrophobic, aliphatic amino acid such as glycine (G), alanine (A), leucine (L), isoleucine (I), methionine (M), or valine (V), a hydrophobic, aromatic amino acid, such as phenylalanine (F), tryptophan (W), or tyrosine (Y) or a non-classical amino acid (e.g. selenocysteine, pyrrolysine, N-formylmethionine β-alanine, GABA and δ-Aminolevulinic acid, 4-Aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

In embodiments, the amino acid mutations are amino acid substitutions, and may include conservative and/or non-conservative substitutions. “Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. As used herein, “conservative substitutions” are exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. As used herein, “non-conservative substitutions” are exchanges of an amino acid by another amino acid listed in a different group of the six standard amino acid groups (1) to (6) shown above.

### Polynucleotides, Vectors, Host Cells

In embodiments, the present disclosure provides a composition comprising a polynucleotide comprising a nucleic acid sequence encoding any one of the amino acids, peptides, antibodies, bispecific molecules and/or compositions described herein. In embodiments, the polynucleotide is RNA or DNA. In embodiments, the RNA is a messenger RNA (mRNA) or a modified mRNA.

In embodiments, the present disclosure provides a composition comprising a vector composition comprising any one of the polynucleotides described herein.

In embodiments, the present disclosure provides a composition comprising a host cell comprising the polynucleotide described herein or the vector described herein. Cells may be cultured in vitro or genetically engineered, for example. Host cells can be obtained from normal or affected subjects, including healthy humans, private laboratory deposits, public culture collections such as the American Type Culture Collection, or from commercial suppliers.

In embodiments, the present disclosure provides a pharmaceutical composition comprising the composition described herein, and a pharmaceutically acceptable excipient or carrier.

In addition, the present disclosure also provides pharmaceutical compositions that comprise compositions as described herein, in combination with a pharmaceutically acceptable carrier. A “pharmaceutically acceptable carrier” (also referred to as an “excipient” or a “carrier”) is a pharmaceutically acceptable solvent, suspending agent, stabilizing agent, or any other pharmacologically inert vehicle for delivering one or more therapeutic compounds to a subject (e.g., a mammal, such as a human, non-human primate, dog, cat, sheep, pig, horse, cow, mouse, rat, or rabbit), which is nontoxic to the cell or subject being exposed thereto at the dosages and concentrations employed. Pharmaceutically acceptable carriers can be liquid or solid, and can be selected with the planned manner of administration in mind so as to provide for the desired bulk, consistency, and other pertinent transport and chemical properties, when combined with one or more of therapeutic compounds and any other components of a given pharmaceutical composition. Typical pharmaceutically acceptable carriers that do not deleteriously react with amino acids include, by way of example and not limitation: water, saline solution, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate). Pharmaceutically acceptable carriers also include aqueous pH buffered solutions or liposomes (small vesicles composed of various types of lipids, phospholipids and/or surfactants which are useful for delivery of a drug to a mammal). Further examples of pharmaceutically acceptable carriers include buffers such as phosphate, citrate, and other organic acids, antioxidants such as ascorbic acid, low molecular weight (less than about 10 residues) polypeptides, proteins such as serum albumin, gelatin, or immunoglobulins, hydrophilic polymers such as polyvinylpyrrolidone, amino acids such as glycine, glutamine, asparagine, arginine or lysine, monosaccharides, disaccharides, and other carbohydrates including glucose, mannose or dextrins, chelating agents such as EDTA, sugar alcohols such as mannitol or sorbitol, salt-forming counterions such as sodium, and/or nonionic surfactants such as TWEEN™, polyethylene glycol (PEG), and PLURONICS™.

Pharmaceutical compositions can be formulated by mixing one or more active agents with one or more physiologically acceptable carriers, diluents, and/or adjuvants, and optionally other agents that are usually incorporated into formulations to provide improved transfer, delivery, tolerance, and the like. A pharmaceutical composition can be formulated, e.g., in lyophilized formulations, aqueous solutions, dispersions, or solid preparations, such as tablets, dragees or capsules. A multitude of appropriate formulations can be found in the formulary known to all pharmaceutical chemists: Remington's Pharmaceutical Sciences (18th ed, Mack Publishing Company, Easton, PA (1990)), particularly Chapter 87 by Block, Lawrence, therein. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies as described herein, provided that the active agent in the formulation is not inactivated by the formulation and the formulation is physiologically compatible and tolerable with the route of administration. See, also, Baldrick, Regul Toxicol Pharmacol 32:210-218, 2000; Wang, Int J Pharm 203:1-60, 2000; Charman J Pharm Sci 89:967-978, 2000; and Powell et al. PDA J Pharm Sci Technol 52:238-311, 1998), and the citations therein for additional information related to formulations, excipients and carriers well known to pharmaceutical chemists.

Pharmaceutical compositions include, without limitation, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

Compositions and formulations can contain sterile aqueous solutions, which also can contain buffers, diluents and other suitable additives (e.g., penetration enhancers, carrier compounds and other pharmaceutically acceptable carriers). Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions provided herein, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the polypeptide components within the compositions provided herein. The formulations can be sterilized if desired.

In embodiments, a composition containing a composition as provided herein can be in the form of a solution or powder with or without a diluent to make an injectable suspension. The composition may contain additional ingredients including, without limitation, pharmaceutically acceptable vehicles, such as saline, water, lactic acid, mannitol, or combinations thereof, for example.

Any appropriate method can be used to administer a composition as described herein to a mammal. Administration can be, for example, parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). In embodiments, administration can be topical (e.g., transdermal, sublingual, ophthalmic, or intranasal), pulmonary (e.g., by inhalation or insufflation of powders or aerosols), or oral.

### Methods of Use

In aspects, there is provided a method of delivery of a drug product/therapeutic payload to a cell by internalization comprising contact a cell, in vitro, ex vivo, or in vivo, with a composition comprising the drug product/therapeutic payload linked to an internalization peptide/cellular uptake peptide, wherein the composition optionally comprises one or more cleavage sites which allow for site-specific removal of or more components of the present compositions when they are inside of a cell.

In aspects, there is provided a method of organelle-specific delivery of a drug product/therapeutic payload comprising contact a cell, in vitro, ex vivo, or in vivo, with a composition comprising the drug product/therapeutic payload linked to an internalization peptide/cellular uptake peptide and an organelle uptake peptide, wherein the composition optionally comprises one or more cleavage sites which allow for site-specific removal of or more components of the present compositions when they are inside of a cell.

In embodiments, there is provided a method of delivery of a drug product/therapeutic payload to a cell by internalization comprising contact a cell, in vitro, ex vivo, or in vivo, with a composition comprising the drug product/therapeutic payload linked to a GPNMB recognition domain, wherein the composition optionally comprises one or more cleavage sites which allow for site-specific removal of or more components of the present compositions when they are inside of a cell.

In aspects and embodiments, the composition of the present disclosure binds to GPNMB expressed on a cell. In embodiments, the composition of the present disclosure reduces, abrogates, or disrupts expression of GPNMB on a cell, optionally a cancer stem cell. In embodiments, the cell is a cancer stem cell or metastatic tumor cell.

In embodiments, the composition of the present disclosure prevents tumors from metastasizing and/or growing by inhibiting expression of GPNMB on the cancer cell.

In embodiments, the composition of the present disclosure reduces, abrogates, or disrupts tumor invasion, metastasis, angiogenesis in triple-negative breast cancer.

In embodiments, the present disclosure provides a method for inhibiting or reducing tumor growth and/or inducing or increasing immune tolerance comprising administering an effective amount of composition of any one of the present embodiments to a patient in need thereof or contacting cells with a composition of any one of the present embodiments (e.g. ex vivo, e.g. with allogeneic or autologous cells).

In aspects, there is provided a method for treating or preventing a disease, comprising administering an effective amount of the present compositions or pharmaceutical compositions thereof to a patient in need thereof, wherein the disease is treatable by targeting a cancer cell surface receptor or a microvascular endothelial cell surface receptor. In embodiments, the disease is treatable by targeting tumors, optionally atypical teratoid rhabdoid tumors.

In aspects, there is provided a method for treating or preventing a cancer. In embodiments the cancer is a solid tumor or a blood cancer.

Tumor cells, or cancer cells refer to an uncontrolled growth of cells or tissues and/or an abnormal increased in cell survival and/or inhibition of apoptosis which interferes with the normal functioning of bodily organs and systems. For example, tumor cells include benign and malignant cancers, polyps, hyperplasia, as well as dormant tumors or micrometastases.

In embodiments, the cancer is, but is not limited to basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

In embodiments, the disease is treatable by targeting pediatric tumors.

In embodiments, the disease is treatable by targeting neural tumors.

In embodiments, the disease is treatable by targeting pediatric neural tumors.

In embodiments, the composition is administered using convection-enhanced delivery.

### Kits

The present disclosure also provides for kits containing the present compositions, or the compositions and one or more other addition, in one or more containers.

Kits containing the pharmaceutical compositions of the invention are also provided in embodiments.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

This disclosure is further illustrated by the following non-limiting examples.

### EXAMPLES

### Example 1: Discovery of Peptides and Biological Assays

### Discovery of Peptides

Cell penetrating peptides were discovered based on determination of an ability to internalize into a cell using various assays, depending on whether a target was known or unknown.

### Biological Assays

Biological activity of the peptides was determined using various assays. To determine proliferative activity, cells were plated at 5×10³ cells per well in a 96-well microtiter plate. Samples and controls were added and the plates incubated for 24, 48 and 72 hours at 37° C. Viability was determined by adding WST-1 and reading at 450 nM over 1-4 hours. Alternatively, viability was determined using a kit such as the EZViable calcein AM cell viability assay (BioVision) or the Live/Dead Cell imaging kit (ThermoFisher). Induction of cell events such as apoptosis was determined using reagents such as Cell Event Caspase 3/7 reagent (ThermoFisher).

FIG. 1 shows sequences of 429 (IGF-1r peptide) and OV-8 (isolated from Ovcar-3 cells) used herein.

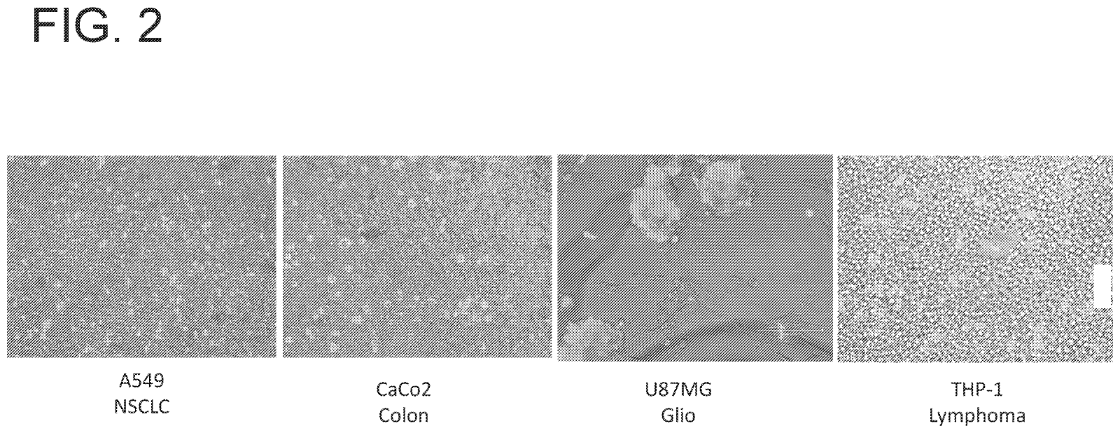

FIG. 2 shows internalization of OV-8 in various cell lines. Skwx-OV-8 has been shown to internalize into a number of cancer cell lines of different lineages.

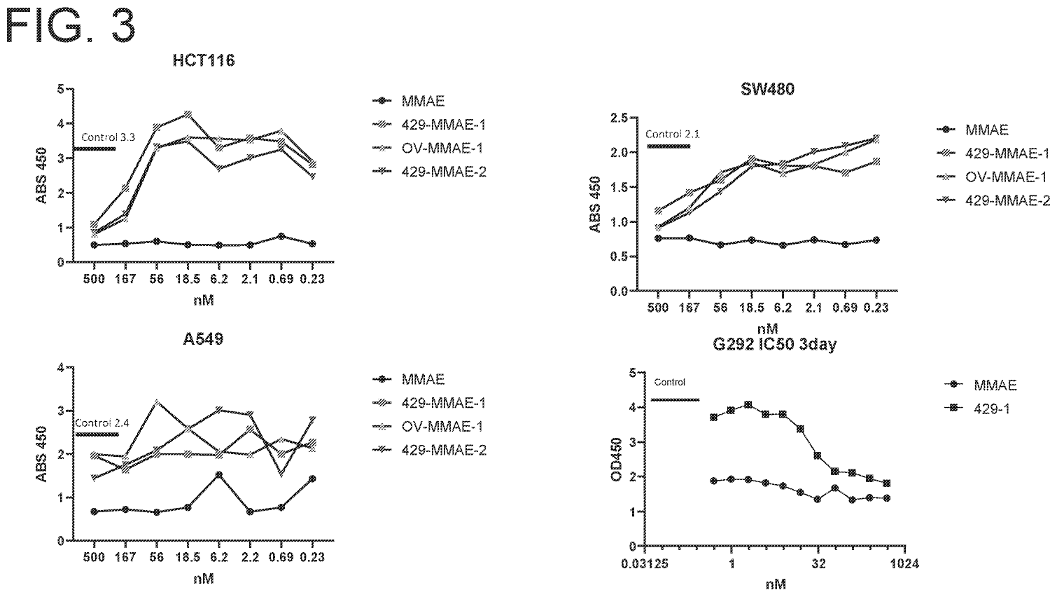

FIG. 3 shows dose response curves for various cell lines with the peptide drug conjugates (“PDCs”): (1) A549: human lung carcinoma, (2) SW480: human colorectal carcinoma, (3) HCT116: human colorectal carcinoma, and (4) G292: human osteosarcoma. Cells were plated at 5000 cells/well in 100 ul D10 on day 0.100 ul of PDCs were added and titrated 1:1 in D10 (starting concentration=500 nM). WST-1 was added at 72 hours and the plates read over 5 hours at 450 nM in a Promega GloMax.

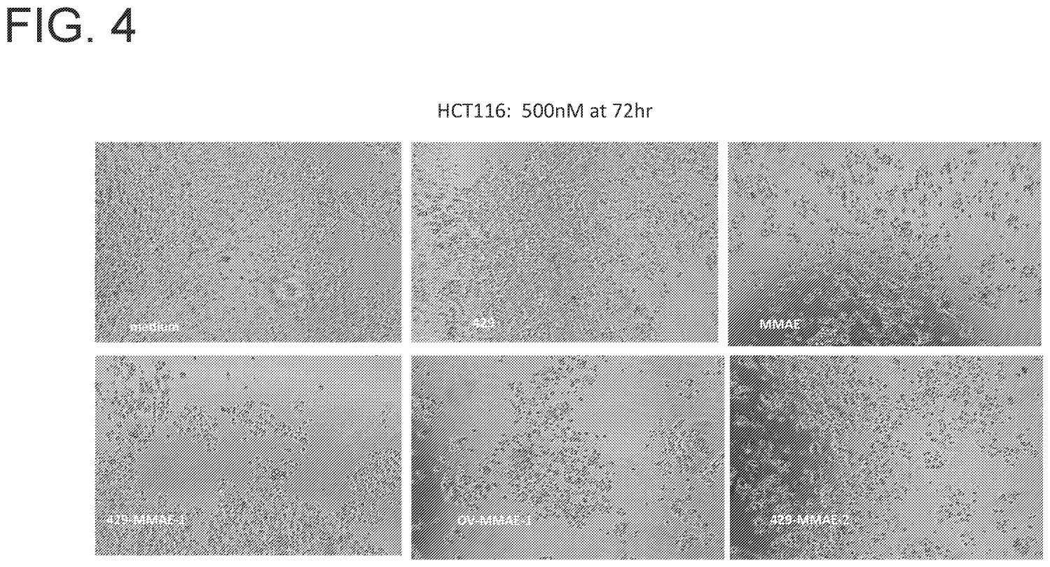

FIG. 4 shows phase contrast photographs of HCT116 including medium control, peptide 429 without MMAE and test samples at 500 nM. This figure shows that the peptide-drug conjugates kill tumor cells.

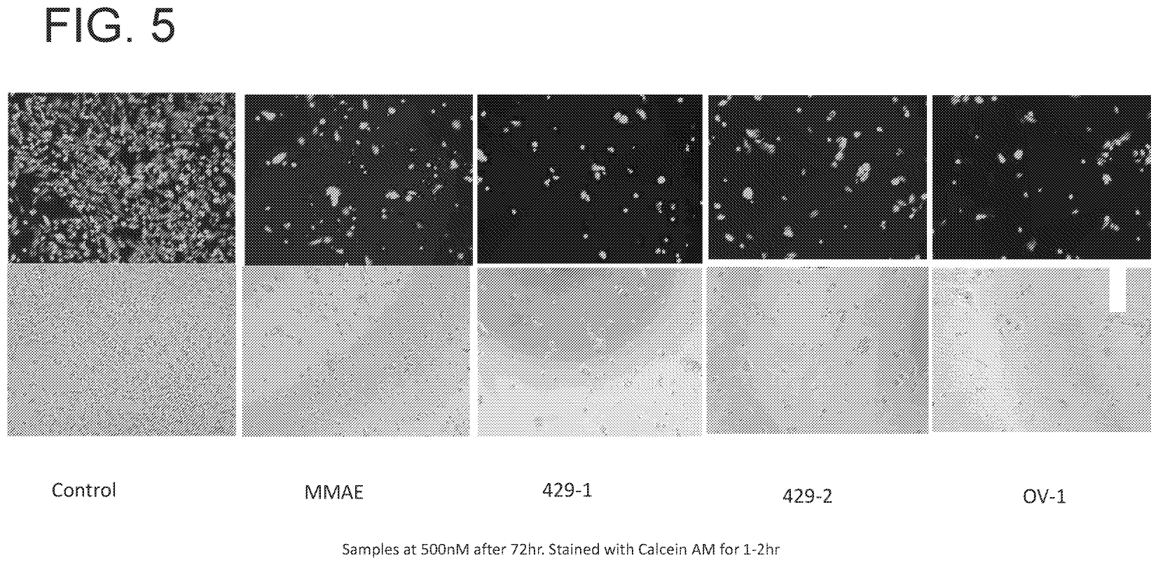

FIG. 5 shows the effects of PDCs on G292 cells. Cells were plated as in FIG. 3 and treated with calcein AM for 1-2 hours at 37° C.

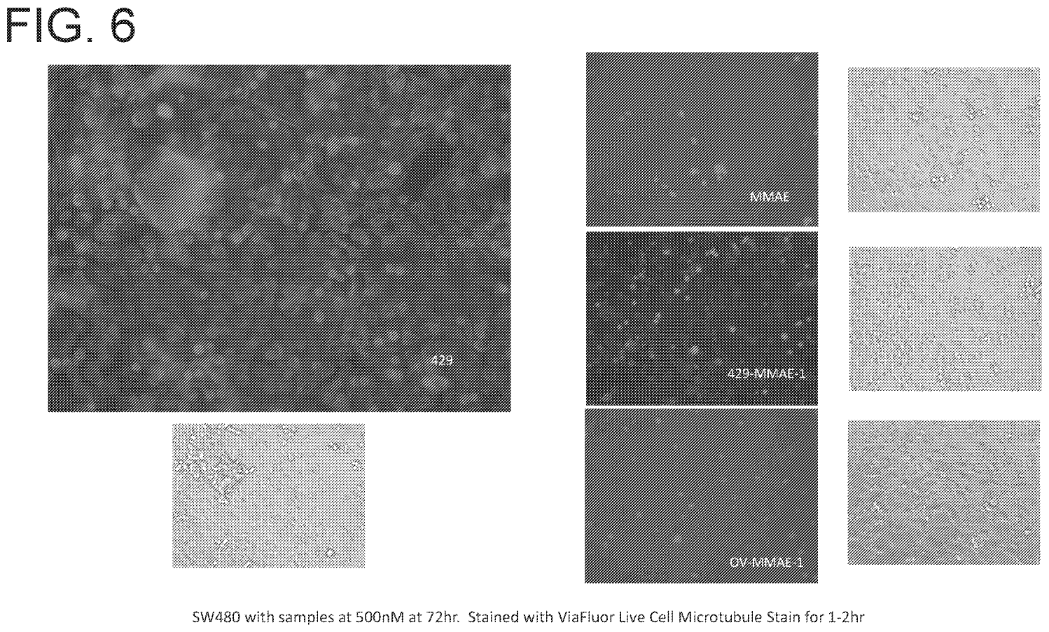

FIG. 6 shows that MAE and PDCs disrupt microtubules. Cells were stained with ViaFluor 647 live cell microtubule stain (Biotium). Cells were treated as in FIG. 3 and stain with ViaFluor for 60 minutes at 37° C.

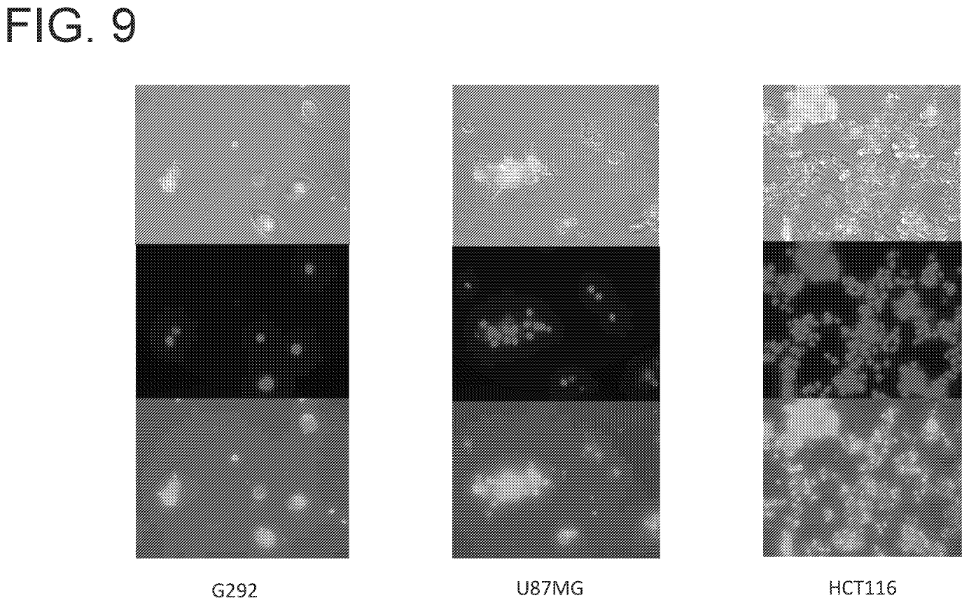

FIG. 9 shows nuclear localization of the 429 construct in 3 cells lines (G292 [osteosarcoma], U87MG [glioblastoma] and HCT116 [colorectal cancer]).

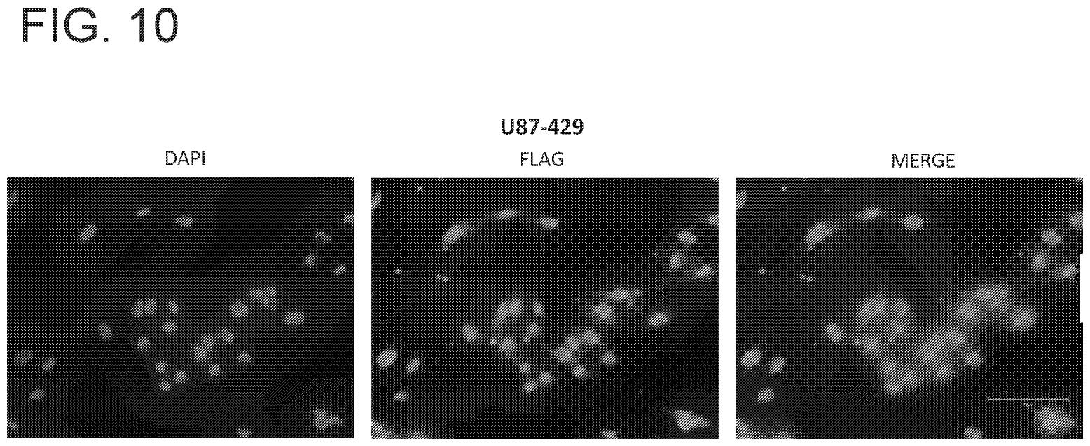

FIG. 10 shows intra-nuclear staining detected with anti-FLAG antibody. As can be seen, the construct does not appear to have been cleaved but still accumulates within the nucleus of U87 cells, i.e., the entire 429 construct can localize to the nucleus.

FIG. 11 shows immunofluorescence data demonstrating that the entire 429 construct localized to the nucleus. Cells were stained with 429-MMP9-NLS-FITC for 3-4 hours at 37° C. and visualized.

### Example 2: Identifying Drug Conjugate for Animal Proof of Concept Studies

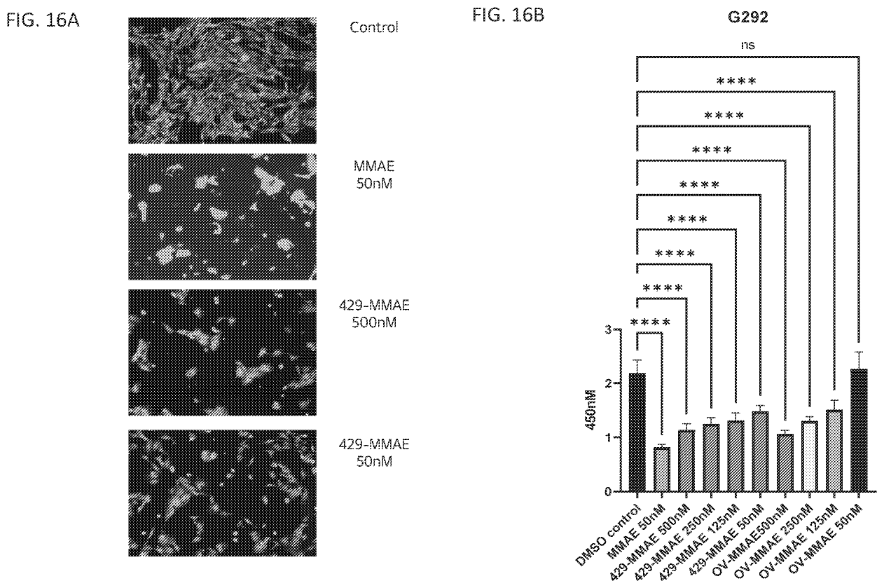

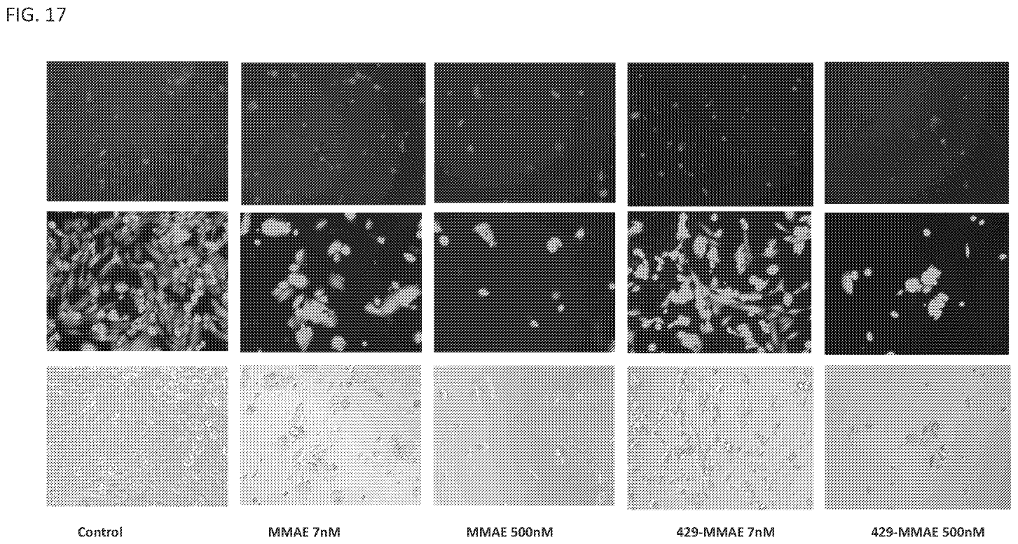

A pocket biologic (peptide or antibody) having an internalization portion, a linker, and a localization signal was developed to deliver a drug or biological therapeutic into the cell. Drugs or biological therapeutics are attached to the localization signal and delivered to the site of interest with a nuclear localization signal. 429-MMAE-1 and 429-NLS-Panobinostat constructs were developed for in vivo studies. G292 cell lines and A431 cell lines were used in the in vivo studies. G292 cells were derived from a primary osteosarcoma of a 9 year old Caucasian female. Cells exhibited the type B phenotype for G6PD and the karyotype is subtetraploid with chromosome markers. A431 cells were established from an epidermoid carcinoma in the skin (epidermis) of an 85-year-old female patient. Epidermal growth factor (EGF) stimulation of A431 cells induces rapid tyrosine phosphorylation of intracellular signaling proteins which control cellular processes such as growth, proliferation and apoptosis. 429-MMAE had an effect in various cell lines (FIG. 16A-FIG. 22B). 429-MMAE affected cell viability in G292 cell lines (FIG. 16A-FIG. 17). 429-MMAE at 50 nm, 125 nm, 250 nm, 500 nm concentrations and OV-MMAE conjugates at 50 nm, 125 nm, 250 nm, 500 nm concentrations were compared to DMSO and MMAE at 50 nm in G292 cells. G292 cells treated with various dosages of 429-MMAE had significantly lower cell viability compared to treatment with DMSO (FIG. 16A-16B). Stained G292 cells treated with 429-MMAE were viewed under a confocal fluorescent microscope and the image showed that 429-MMAE affected microtubules in G292 cells (FIG. 17).

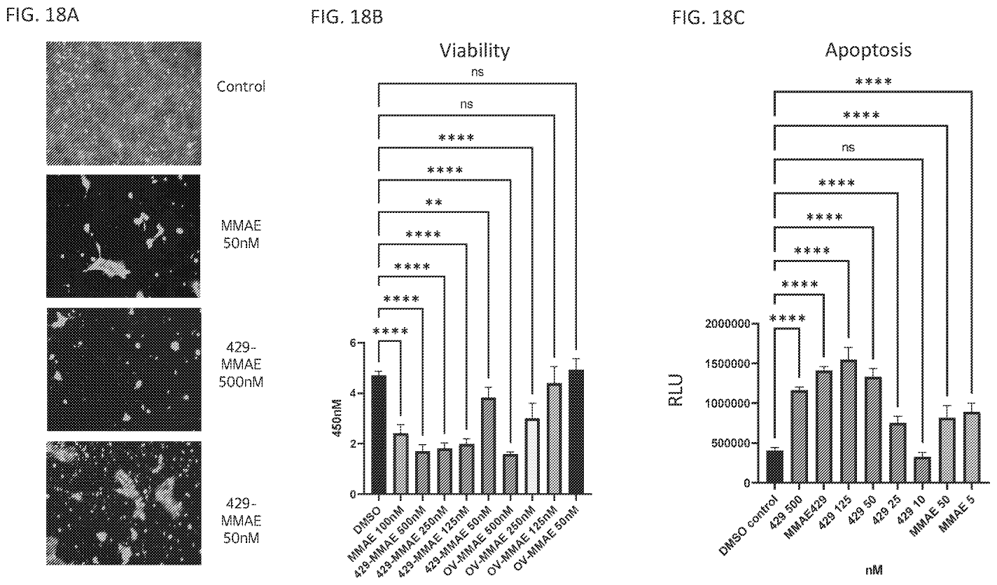

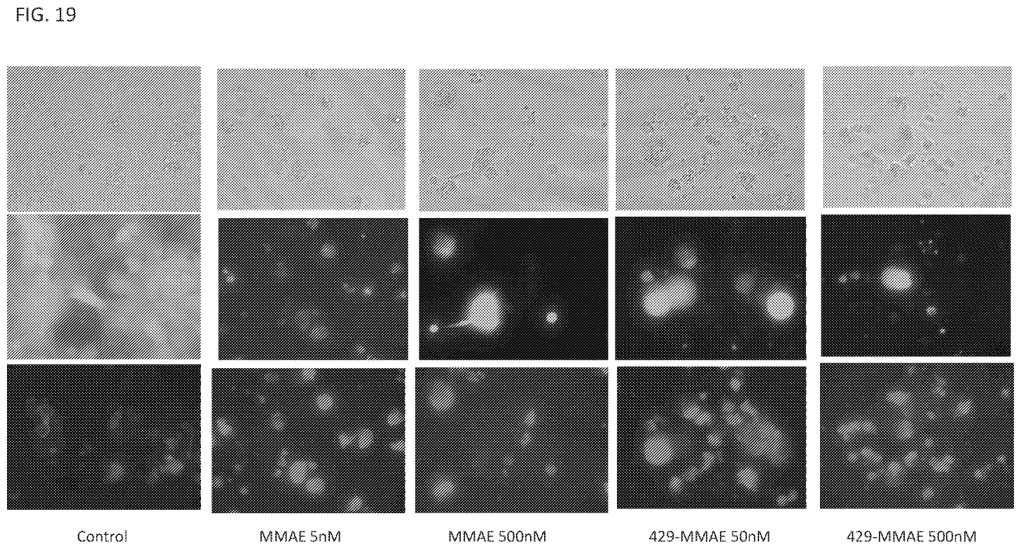

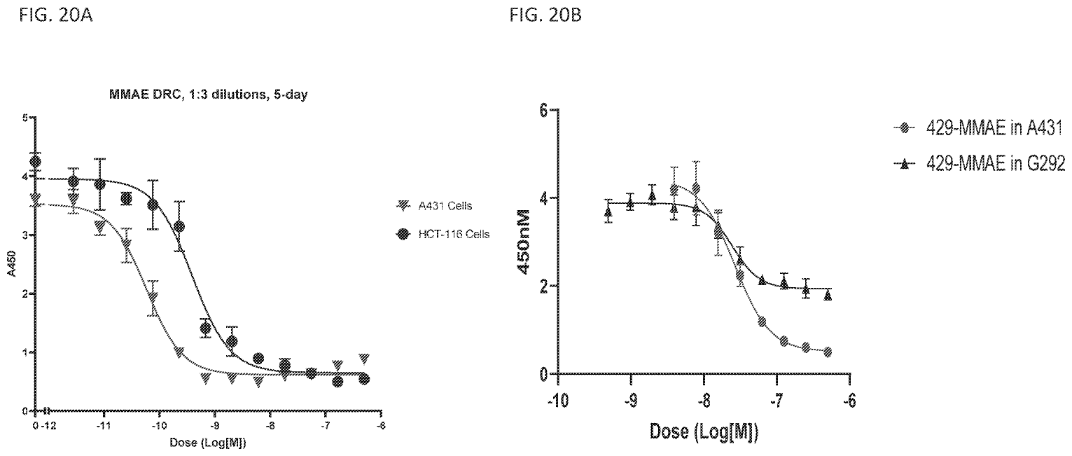

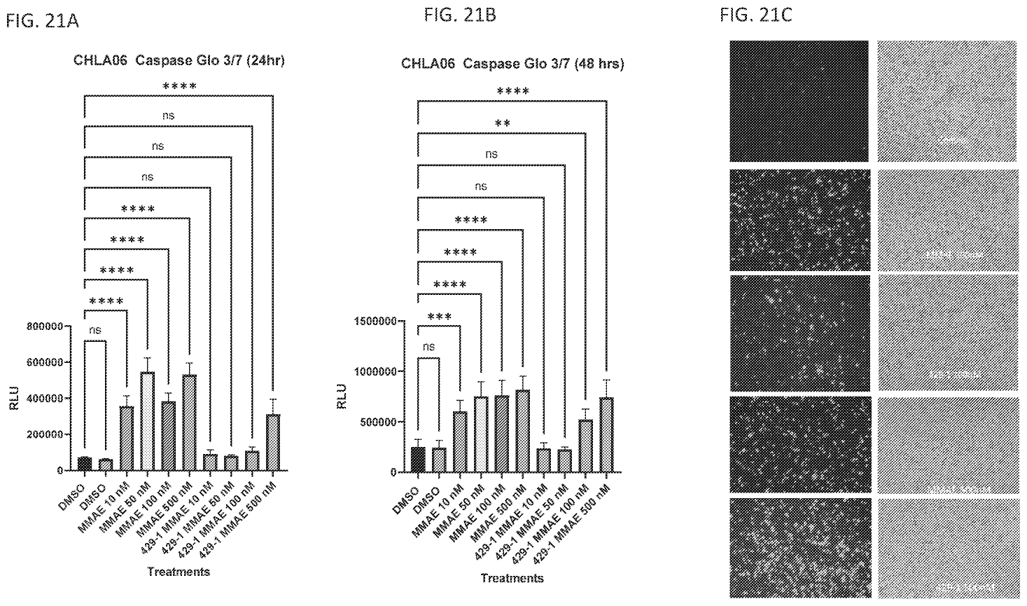

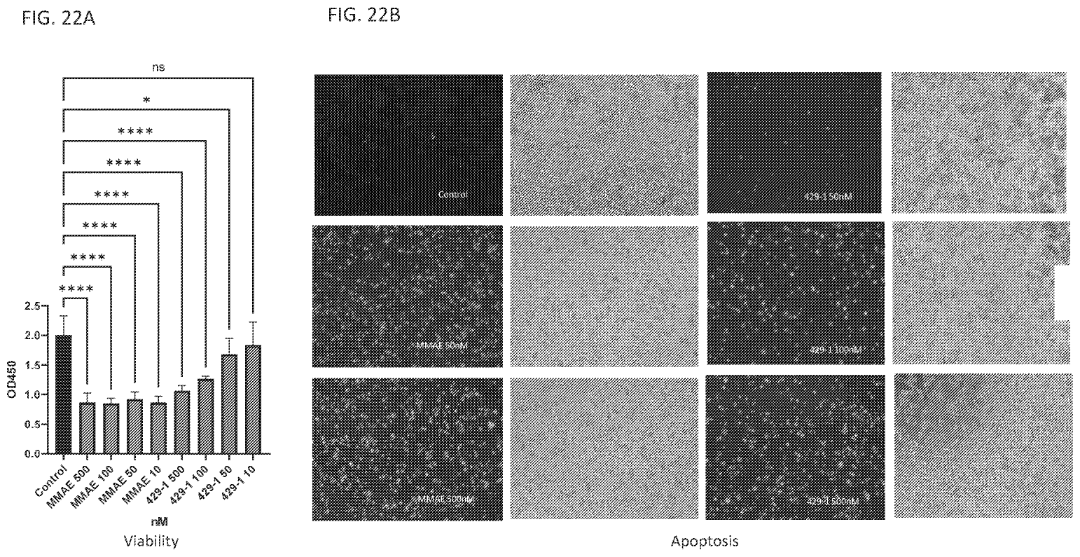

429-MMAE also affected viability and apoptosis in A431 cells (FIG. 18A-FIG. 19). 429-MMAE at 50 nm, 125 nm, 250 nm, 500 nm concentrations and OV-MMAE at 50 nm, 125 nm, 250 nm, 500 nm concentrations were compared to DMSO and MMAE at 50 nm in A431 cells. A431 cells treated with various dosages of 429-MMAE had significantly lower cell viability compared to treatment with DMSO (FIG. 18B). A431 cells treated with various dosages of 429-MMAE had significantly higher cell apoptosis compared to treatment with DMSO (FIG. 18C). 429-MMAE also disrupted microtubules in A431 cells (FIG. 19). Dose response curves of MMAE at 1:3 dilutions were compared between A431 cells and HCT116 cells (FIG. 20A). A431 cells had an IC₅₀ value (four-parameter non-linear regression) of 71.2 pM. HCT-116 cells had an IC₅₀ value of 500.4. Dose response curves of 429-MMAE in A431 and G292 were compared (FIG. 20B). A431 cells had an IC₅₀ value of 26.4 nM. G292 cells had an IC₅₀ value of 24.1 nM. 429-MMAE induced apoptosis in CHLA06 cells as shown in caspase assays (FIG. 21A-FIG. 22B). 429-1 MMAE at 500 nM treatment induced more apoptosis in CHLA06 cells compared to DMSO at 24 hours (FIG. 21A). 429-1 MMAE at 100 nM and 500 nM treatments induced more apoptosis in CHLA06 cells compared to DMSO at 48 hours (FIG. 21B). 421-1 MMAE represents 429-MMAE with a drug antibody ratio (DAR) of 1. FIG. 22A compares CHLA06 cell viability after treatments with various dosages of MMAE and 429-1 MMAE.

Without being bound to a particular theory, 429-MMAE has a higher IC₅₀ compared to MMAE alone, indicating that the linker is stable and the drug is not being released. The smaller size of the 429-MMAE conjugate leads to rapid tumor accretion and the expected shorter half-life is a positive for many indications as the goal is to treat and clear the drug as rapidly as possible. 429-MMAE conjugates cost less to manufacture compared to traditional antibody drug conjugates. 429-MMAE conjugates are amenable to treating pediatric neural tumors because drug administration is intratumoral and not systemic.

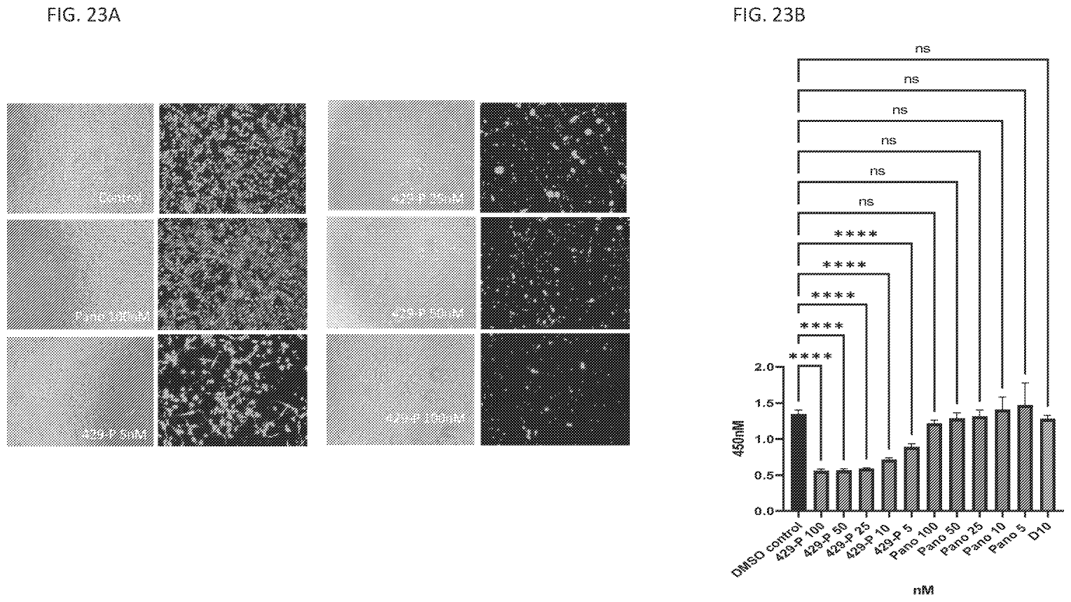

429-Panobinostat constructs can localize to the nucleus of a cell as an uncleaved construct and target atypical teratoid rhabdoid tumors (ATRT). (FIG. 23A-FIG. 24B). 429-NLS-Panobinostat (429-P) inhibits CHLA06 cells. 429-Panobinostat constructs at 100 nm, 50 nm, 25 nm, 10 nm, and 5 nm concentrations significantly affect cell viability compared to DMSO (FIG. 23B). Dose response curves show that 429-Panobinostat constructs are more effective than Panobinostat alone (FIG. 24A-FIG. 24B). IC₅₀ of A431 cells treated with 429-Panobinostat is 32.6 nM and A431 treated with Panobinostat is 108 nM. IC₅₀ of CHLA06 cells treated with 429-Panobinostat is 12.3 nM and A431 treated with Panobinostat is >500 nM. 429-MMAE and 429-NLS-Panobinostat inhibit the AT/RT cell line CHLA06.

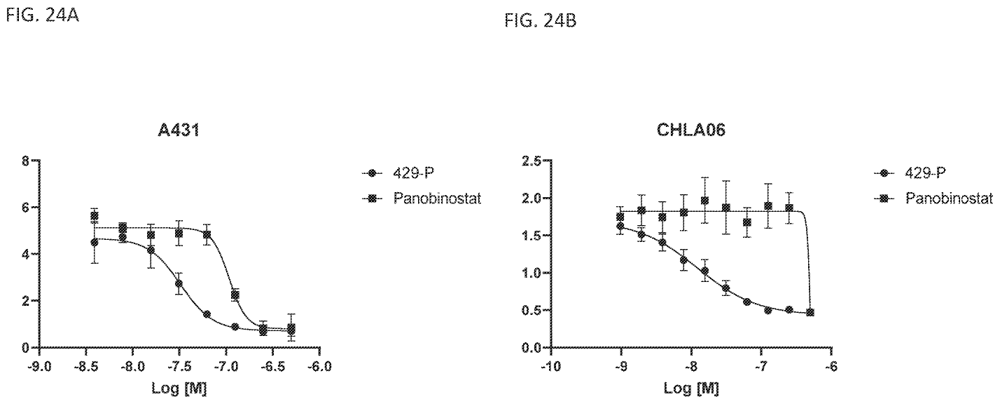

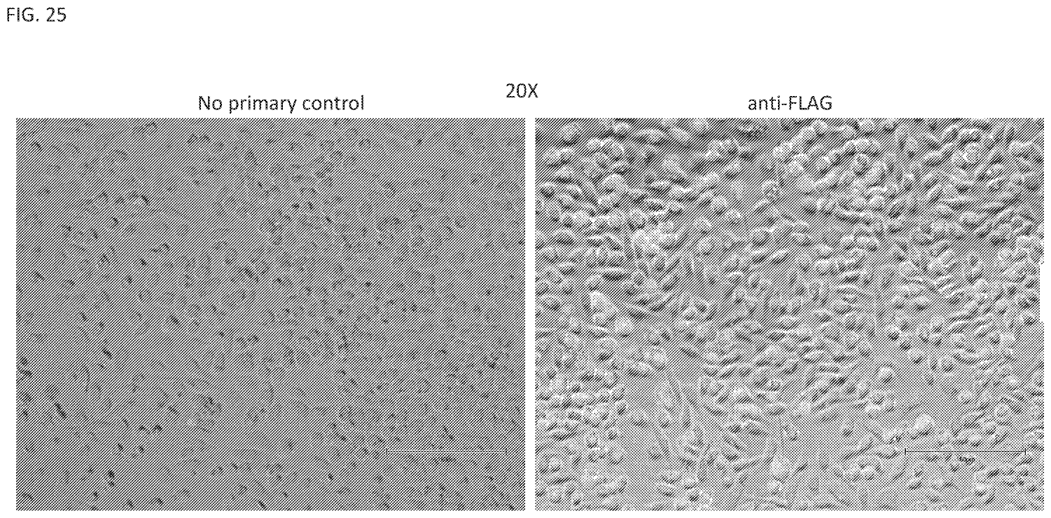

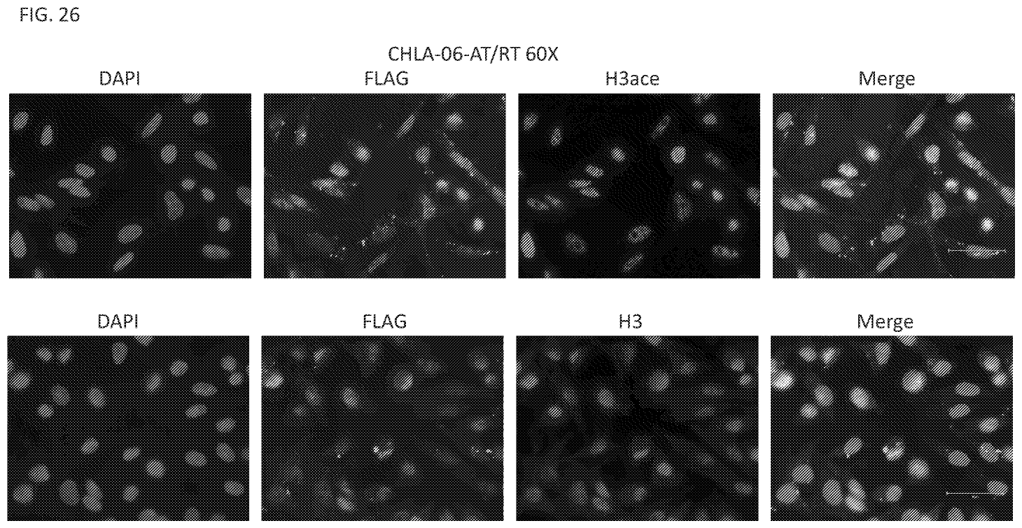

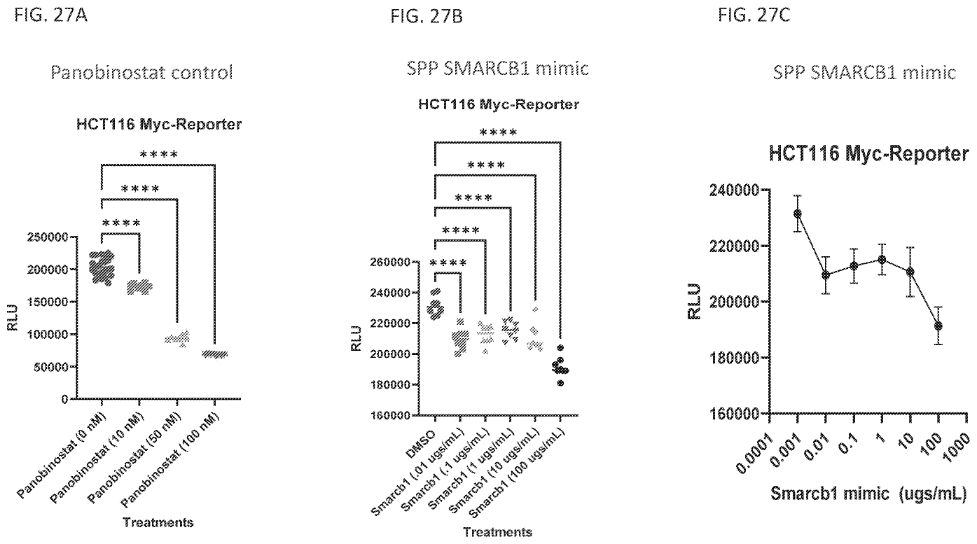

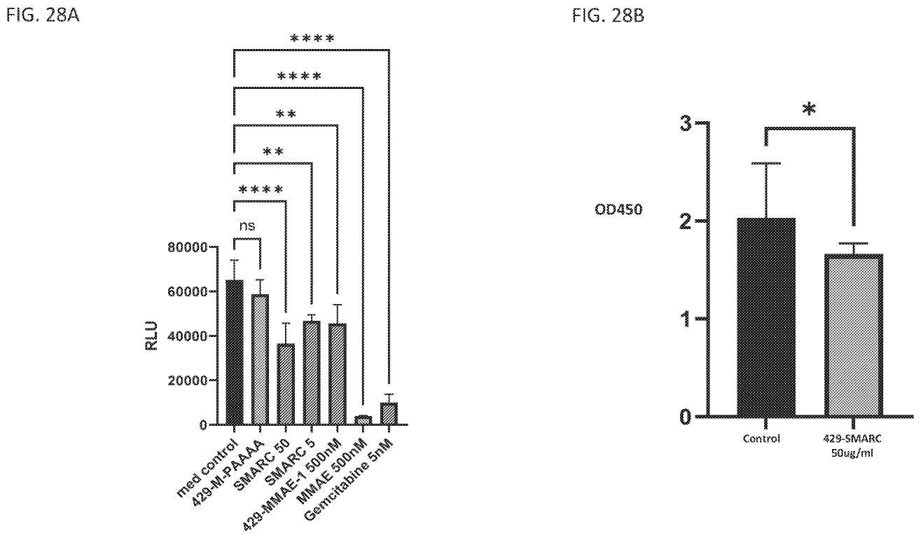

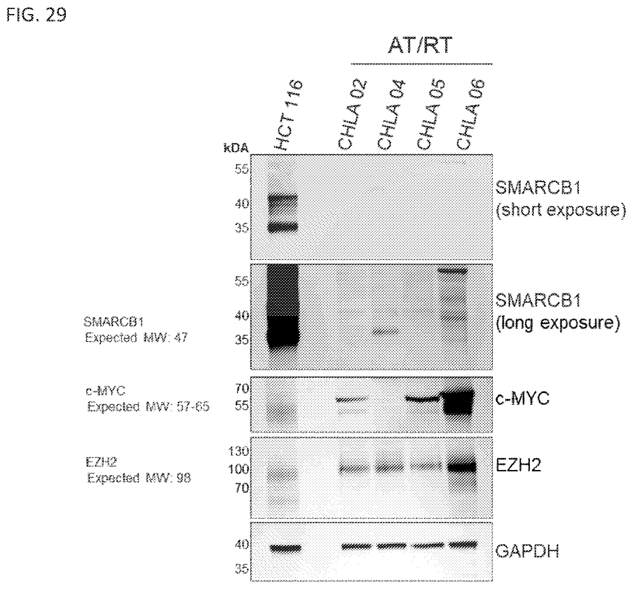

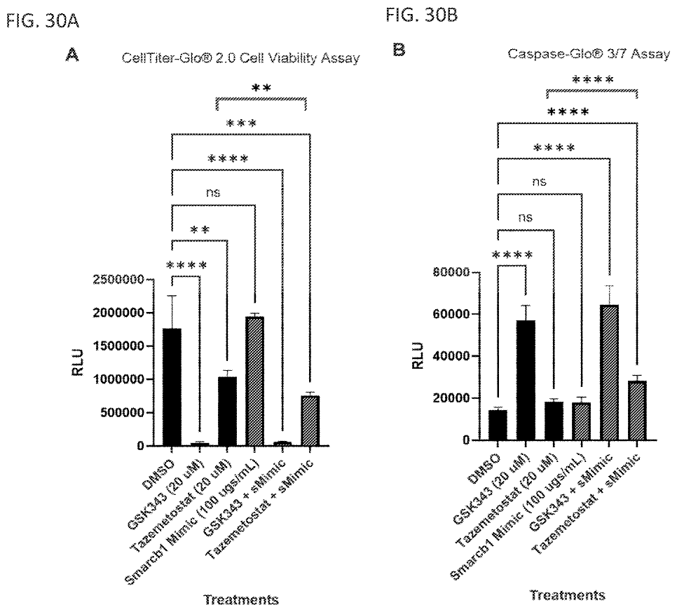

429-SMARCB1 is internalized in CHLA06 cells as shown in a confocal fluorescent microscopy image (FIG. 25). 429-SMARCB1 also co-localizes with histones as shown in a confocal fluorescent microscopy image (FIG. 26). 429-SMARCB1 inhibits Myc transcription. FIG. 27A shows the results of HCT116 Myc-Reporter cells treated with various dosages of Panobinostat. FIG. 27B-FIG. 27C show HCT116 Myc-Reporter cells treated with increasing dosages of 429-SMARCB1, causing a greater inhibition of Myc transcription. 429-SMARCB1 affects both Myc transcription and viability as demonstrated by FIG. 28A-FIG. 28B. 429-SMARCB1 at 50 nM, 429-SMARCB1 at 5 nM, and 429-MMAE-1 at 500 nM all significantly affects Myc transcription as compared to the control (FIG. 28A). 429-SMARCB1 also affects HCT116 cell viability compared to a control (FIG. 28B). 429-SMARCB1 and tazemetostat reduced cell viability and increased caspase activity in CHLA06 cells (FIG. 30A-FIG. 30B).

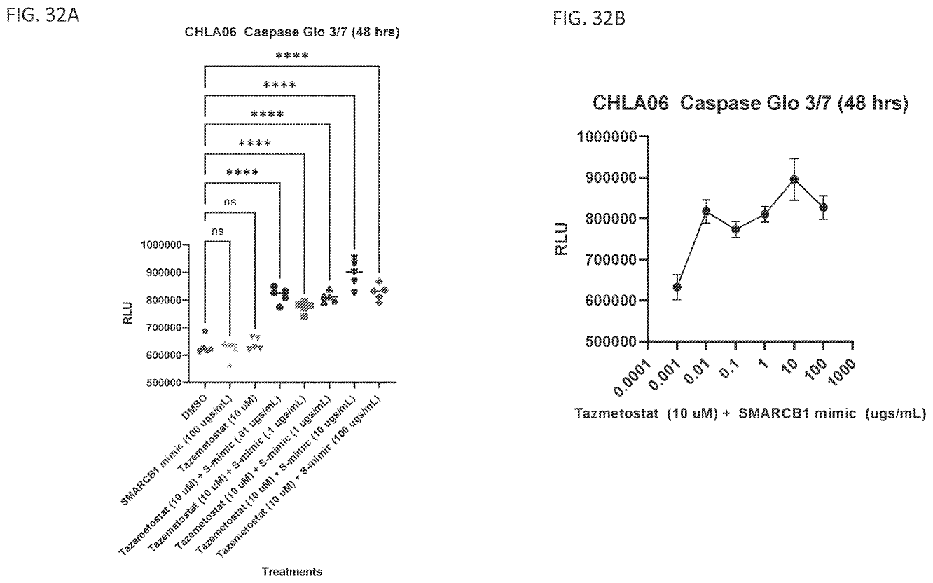

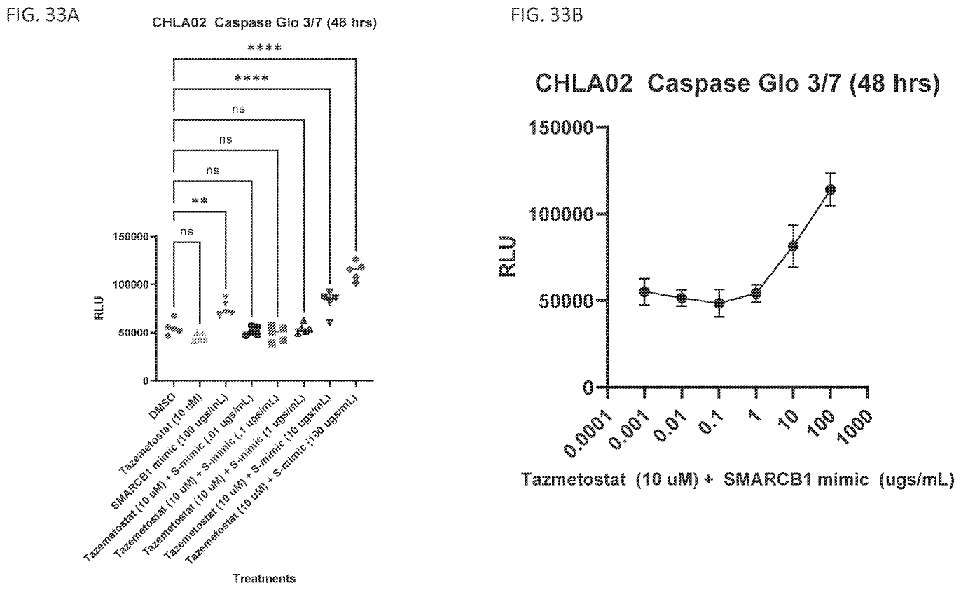

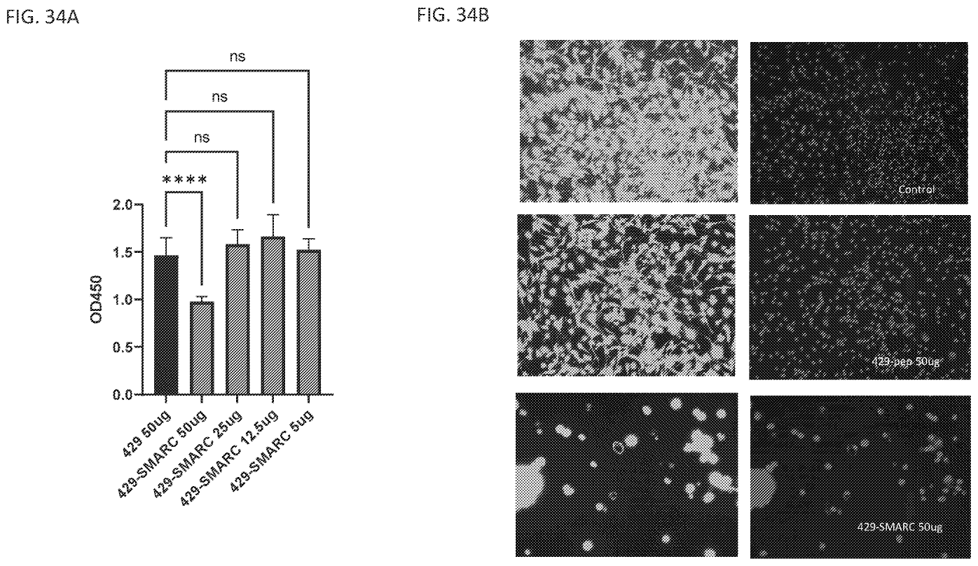

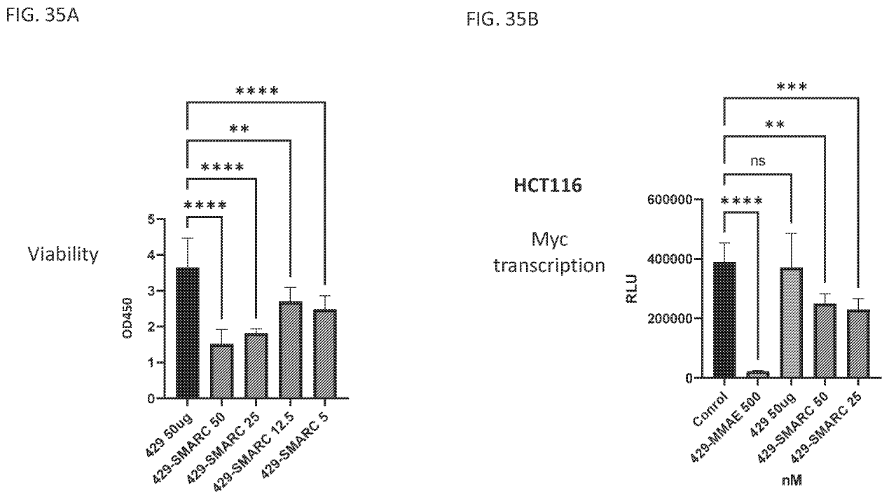

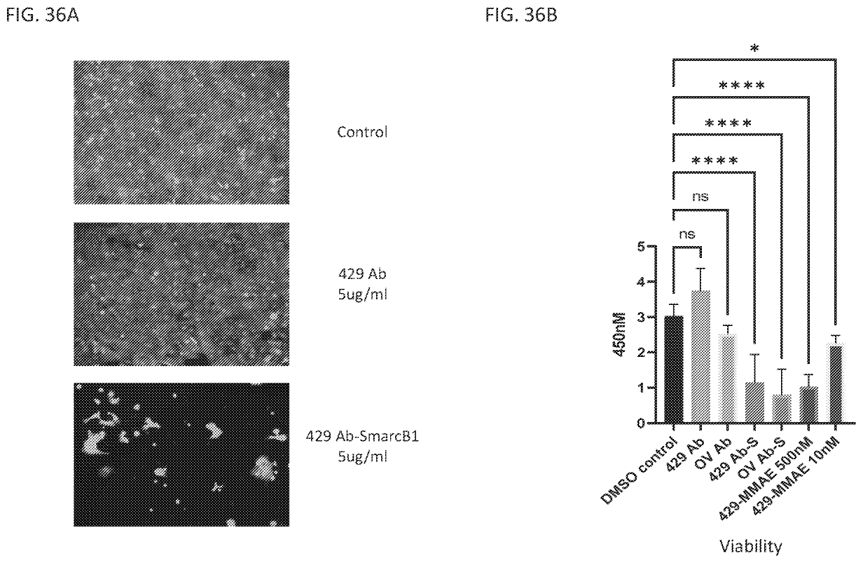

The 429-SMARCB1(F) mimic conjugate structure is shown in FIG. 31. SMARCB1 Mimic (F) and tazemetostat significantly increased caspase activity CHLA06 cells (FIG. 32A-FIG. 32B). SMARCB1 Mimic (F) and tazemetostat significantly increased caspase activity CHL02 cells (FIG. 33A-FIG. 33B). 429-SMARCB1 (F) at 50 ug also significantly affected CHLA06 cell viability compared to 429 control (429 is the delivery peptide without the mimic (FIG. 34A-FIG. 34B)). 429-SMARCB1(F) also inhibited Myc transcription and HCT116 cell viability (FIG. 35A-FIG. 35B).

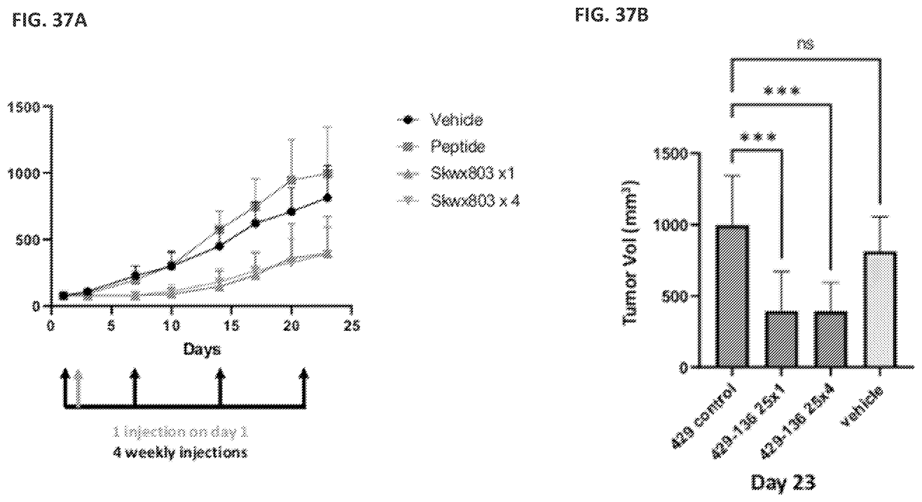

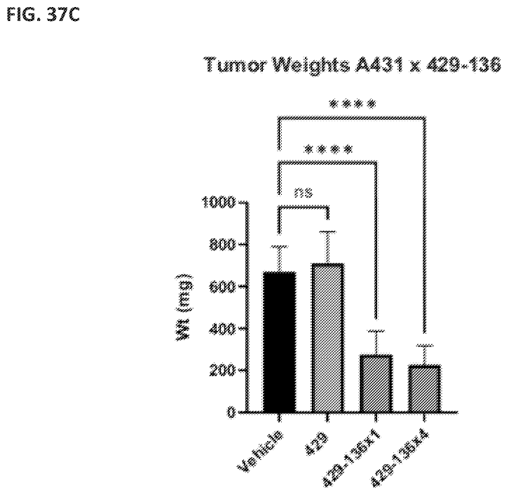

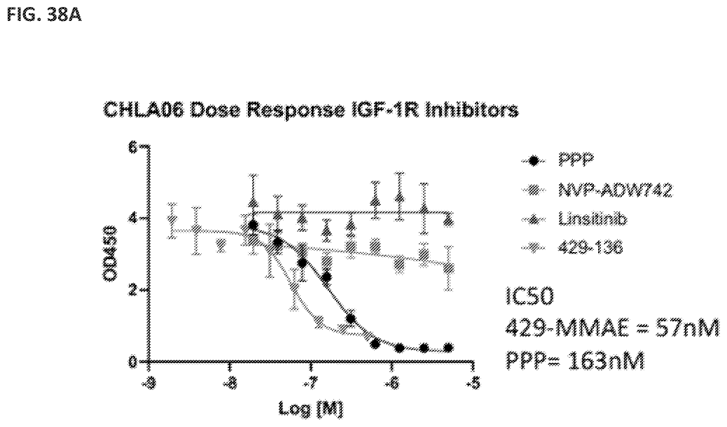

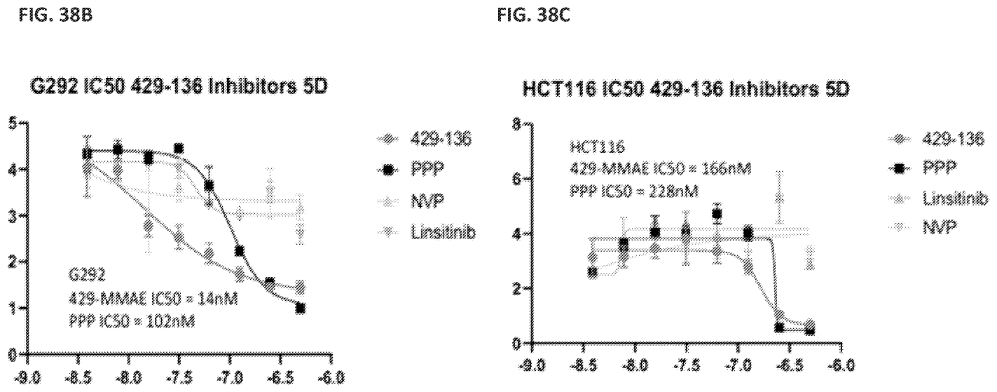

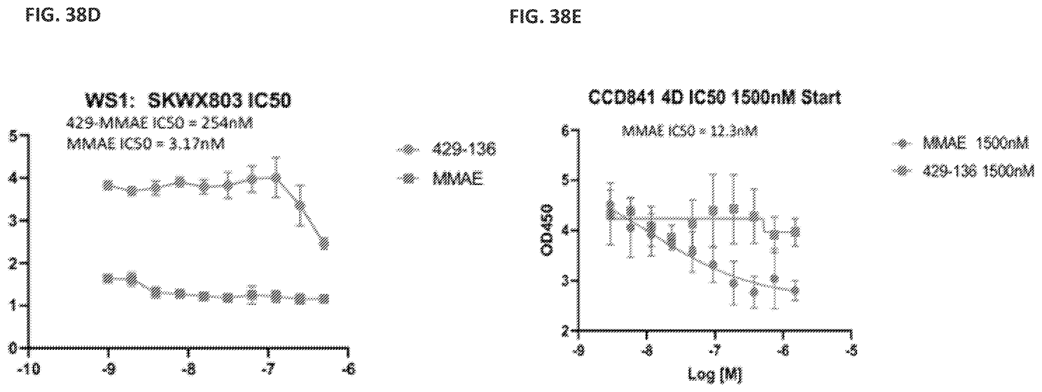

A 429 ScHv-SMARCB1 antibody with the structure of “antibody-429 CDR3-MMP9-NLS-SmarcB1” was also developed. 429 ScHv-SMARCB1 antibody significantly affected cell viability compared to DMSO control (FIG. 37A-FIG. 37B). The 429-antibody and OV-antibody are scHv with the peptides in the CDR3, 429-antibody-S and OV-antibody-S are the scHVs with the SMARCB1 mimic, and 429-MMAE is used as a positive control (FIG. 38B).

Without being bound to a particular theory, the 429-SMARCB1 mimic conjugates induced apoptosis in HCT116, CHLA02 and CHLA06 cells. The 429-SMARCB1 mimic conjugates inhibited myc activation in HCT116 cells. There was a synergistic apoptotic effect when 429-SMARCB1 was used in combination with tazemetostat in ATRT cell lines. The 429-antibody-SMARCB1 construct inhibited the growth of cancer cells.

### Example 3: Testing Drug Conjugates

### 429-MMAE Animal Studies

429-MMAE drug conjugates were shown to inhibit A431 xenografts in nude mice (FIG. 37A-37C). A431 cells were used as xenografts. Animals received 1 injection of vehicle (PBS), 429 peptide (50 ug/injection) or 429-137 (25 ug/injection) or 4 weekly injections of 429-136 (25 ug/injection) by the intra-tumoral route. The tumors were measured twice weekly and the animals sacrificed when tumors reached 1000 mm³. FIG. 37A shows a comparison of tumor volume after treatment of 429-MMAE at the two different dosages (SKWX803 represents 429-MMAE). FIG. 37B shows a comparison of tumor volume on day 23 after treatments of 429-MMAE (429-136 represents 429-MMAE). Animals given 1 injection of 429-MMAE (25 ug/injection; 1 mg/kg) and 4 weekly injections of 429-MMAE (25 ug/injection; 1 mg/kg) showed significantly less tumor growth compared to control vehicle or control 429 peptide. The weight of excised tumors of mice treated with 1 injection of 429-MMAE (25 ug/injection; 1 mg/kg) and 4 weekly injections of 429-MMAE (25 ug/injection; 1 mg/kg) were significantly lighter compared to control vehicle or 429 peptide (FIG. 37C). Animal body weights were consistent throughout the study. There was no apparent toxicity with the 429-MMAE conjugate.

### 429-MMAE Effects on Normal and Cancer Cell Lines

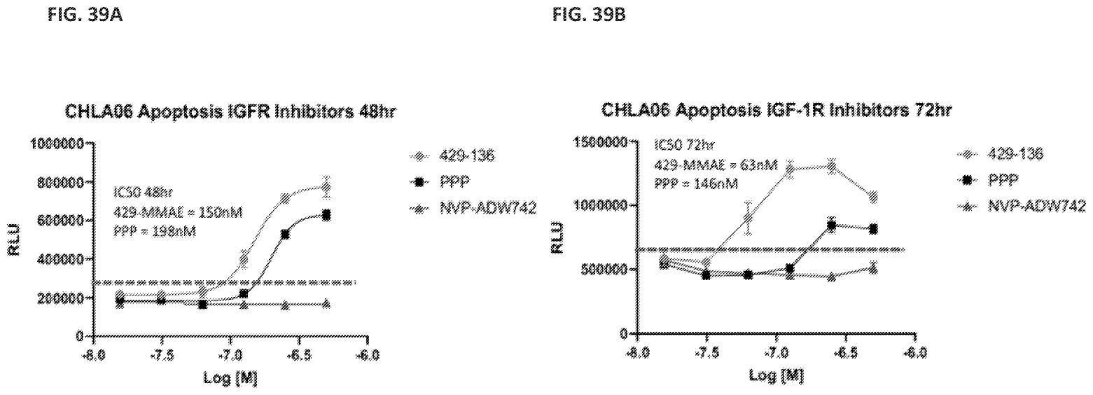

Three small molecule inhibitors of IGF-1R were compared with 429-MMAE on various cell lines (FIG. 38A-38E). FIG. 38A shows a comparison of CHLA06 dose response after treatment with 429-MMAE, PPP (Picropodophyllin), Linsitinib, and NVP-ADW742. Both Linsitinib and NVP-ADW742 had no effects on CHLA06 cells. PPP was active in CHLA06 cells but was less effective compared to 429-MMAE in terms of IC50. Linsitinib, NVP-ADW742, PPP, and 429-MMAE were also tested in G292 and HCT 116 cell lines (FIG. 38B-38C). 429-MMAE was more effective in inhibiting cell growth in both G292 and HCT 116 cell lines compared to the three IGF-1R inhibitors in terms of IC50. 429-MMAE is also better at inducing apoptosis in CHLA06 cells compared to IGF-1R inhibitors at 48 hours and 72 hours after treatment (FIG. 39A-39B).

429-MMAE and MMAE were also tested on two normal cell lines, WS1 and CCD841 (FIG. 38D-38E). 429-MMAE had no tangible effect on CCD841 and was 5-10 fold less efficacious on WS1 cells. WS1 cells were derived from normal human skin. WS1 cells have a doubling potential of 67 population doublings. CCD841 cells were isolated from normal human colon tissue. Without being bound to a particular theory, there appears to be a therapeutic index between 429-MMAE effects on tumor cell lines and normal cells lines which could increase the maximum therapeutic dose in the clinic.

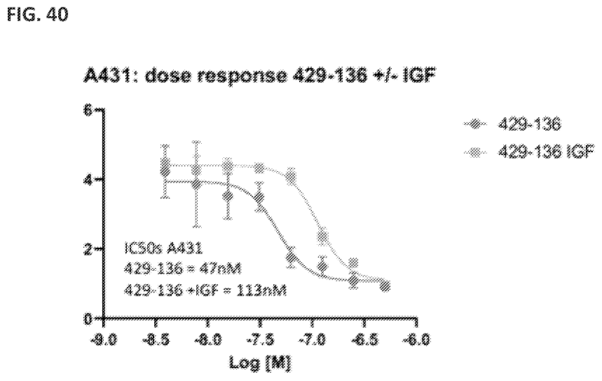

429-MMAE was also tested in the presence or absence of IGF-1 (FIG. 40). FIG. 40 shows a shift in IC50 in A431 cells in the presence of IGF-1. Without being bound to a particular theory, shift between cells in the presence or absence of IGF suggests that IGF-1 is blocking the binding of the conjugate.

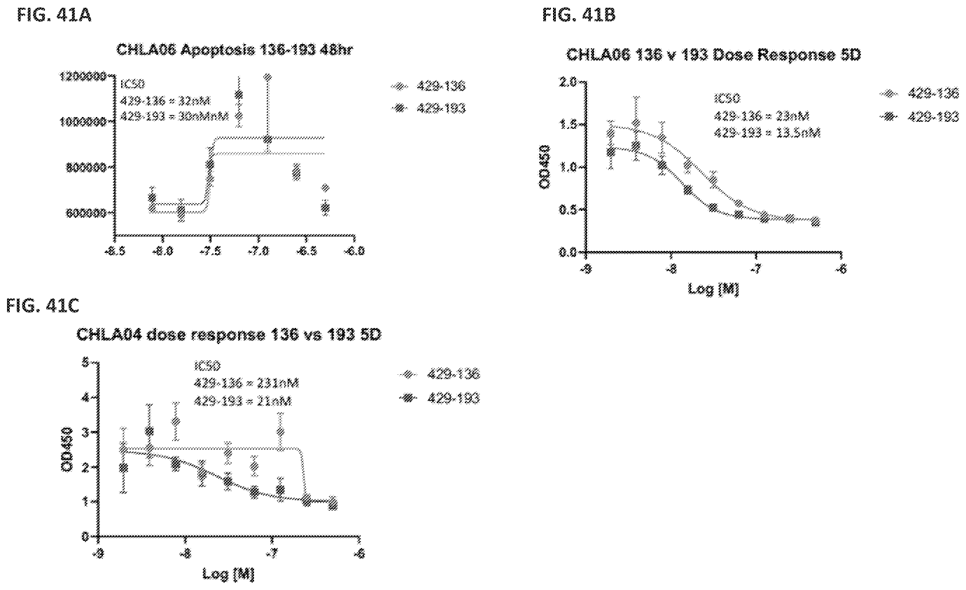

429-MMAE was also tested with different drug peptide ratios (DRP) (i.e., the number of drug molecules per peptide) on CHLA04 and CHLA06 cells (FIG. 41A-41C). 429-136 represents 429-MMAE with a DPR of 1 (average 1.36). 429-193 represents 429-MMAE with a DPR of 2 (average 1.93). The drug linkers are conjugated to cysteine residents. The 429 peptide has 2 cysteines, so the 429 peptide can have a DPR of 1 or 2. CHLA-06 apoptosis, CHLA-06 dose response and CHLA04 dose response were compared between the two 429-MMAE constructs.

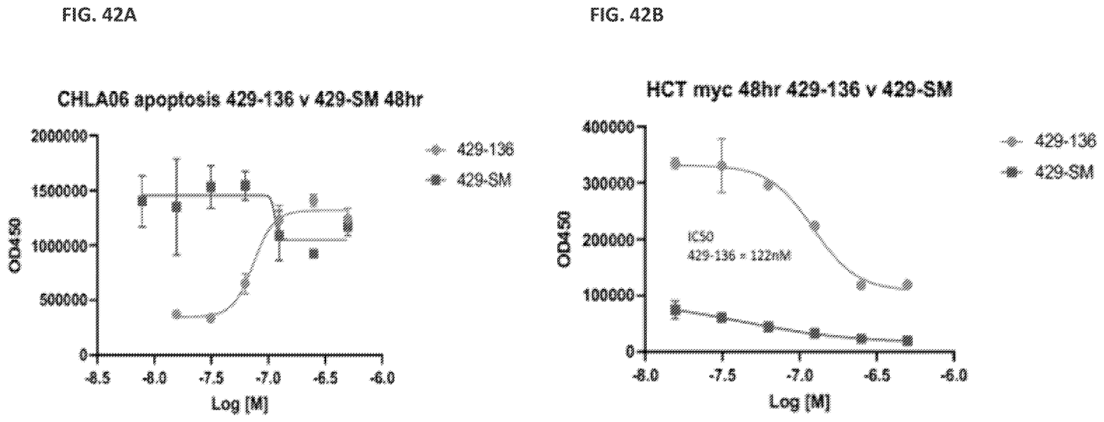

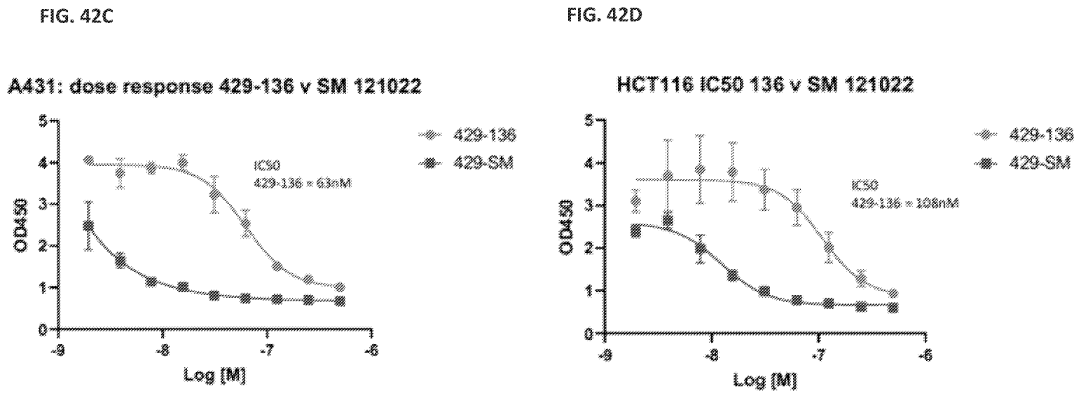

429-MMAE was also compared with 429-SM in various cell lines. 429-SM represents 429-Smarc-MMAE and consists of 429-linker-NLS-SMARCB1 mimic to which 1 or 2 MMAE molecules are conjugated through the cysteines. CHLA06 cells were plated at 10,000 cells/well in B27-5 overnight. Samples were added and incubated for 48 hours at 37° C. Caspase 3/7 reagent was added and plates were read after 30 minutes at room temperature. 429-SM induced apoptosis of CHLA06 cells throughout the entire dose response (FIG. 42A). Induction of apoptosis is directly correlated with drug concentration (i.e., higher the concentration of drug, the higher the apoptotic effect). 429-SM also inhibited HCT myc transcription (FIG. 42B). 429-SM was also effective in A431 and HCT116 cells (FIG. 42C-42D).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

The content of any individual section may be equally applicable to all sections.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any way.

What is claimed is:

1. A composition according to the formula:
J-Y-Z, wherein:
J comprises a cellular uptake peptide and optionally, a cell surface receptor targeting peptide and/or an organelle uptake peptide and/or a protein detection peptide
when there is a cell surface receptor targeting peptide and/or an organelle uptake peptide and/or protein detection peptide, there is optionally a cleavage site between each of these peptides and also between these peptides and the cellular uptake peptide;
the cellular uptake peptide is capable of binding to a marker which is internalized by a cell, optionally expressed on a cancer cell, optionally selected from OVCAR, MIA, A549, A431, G292, HCT116, CHLA02, CHLA04, CHLA05, CHLA06, or
has an amino acid sequence selected from:
(SEQ ID NO: 48)
DFYGCLLDLSLGVPSLGWRRRCITA (″429″);
(SEQ ID NO: 43)
YQWVSWWHRGLLRPVLRGSLKPWYIFVRIHISWGQVHVG,
(SEQ ID NO: 44)
TGWWFFWSYVCSVRLLAVTCSFWCTEQGEESLPRAGTHTH,
(SEQ ID NO: 45)
YTLGLGKSQRWNLFRNFFCYMPRVTVFCKSCRCVSFQTSG,
(SEQ ID NO: 46)
HYLLIRCARGFCEVRLVDAMCSITIVIDCVLFTSQRERAE;
(SEQ ID NO: 47)
QGSLISCWVTFVQESWDAYSLCSDLFKCQGGQVFSICEPE,
(SEQ ID NO: 49)
FSFGNSSLRKLPGWVRRRRCARGVWR (″OV-8″);
(SEQ ID NO: 48)
DFYGCLLDLSLGVPSLGWRRRCITA (″1 429-M″);
(SEQ ID NO: 49)
FSFGNSSLRKLPGWRRRRCARGVWR (″#3 OvB8-M″);
(SEQ ID NO: 48)
DFYGCLLDLSLGVPSLGWRRRCITA (″429-F″);
and
(SEQ ID NO: 49)
FSFGNSSLRKLPGVVRRRRCARGVWR (″#4 OVB8-F″);
or a variant thereof, or
has an amino acid sequence selected from Table 1 or FIG. 1, or a variant thereof;
Z comprises a therapeutic payload; and
Y is a linker covalently attaching J and Z.

2. The composition of claim 1, having the formula:
(V)ₘ-W-(X)ₙ-Y-Z, wherein:
V comprises a cell surface receptor targeting peptide;
m is 0 or 1;
W comprises a cellular uptake peptide;
X comprises an organelle uptake peptide; and
n is 0 or 1.

3. The composition of claim 1 or 2, wherein Z is or comprises 1, or 2, or 3, or 4, or 5, or 6, or 7, or 8, or 9, or 10 or more therapeutic payloads.

4. The composition of claim 1-3, wherein the therapeutic payload is an anticancer agent.

5. The composition of any of claims 1-4, wherein the therapeutic payload is a small molecule drug, biological, or radionuclide.

6. The composition of claim 5, wherein the therapeutic payload is a biological, which is selected from a toxin, an antibody, or an oligonucleotide.

7. The composition of claim 4, wherein the anticancer agent is monomethyl auristatin E (MMAE).

8. The composition of claim 4, wherein the anticancer agent is a histone deacetylase (HDAC) inhibitor.

9. The composition of claim 8, wherein the HDAC inhibitor is selected from vorinostat, panobinostat, tazemetostat, mocetinostat, romidepsin, trichostatin A and trapoxin A.

10. The composition of claim 5, wherein the payload is an antisense oligonucleotide that binds to kras, gemcitabine, or cisplatin.

11. The composition of claim 5, wherein the therapeutic payload is a SmarcB1 mimic.

12. The composition of any one of the above claims, wherein the cellular uptake peptide is capable of binding to Ovcar-3 cells and/or has an amino acid sequence selected from:
(SEQ ID NO: 50)
PRGWRRCLGWSVGPASGSGP,
(SEQ ID NO: 49)
FSFGNSSLRKLPGWRRRRCARGVWR,
(SEQ ID NO: 51)
CLGVCISGDPEQADMWGCRP,
(SEQ ID NO: 52)
ILSRSQYLVFGYAWARGSR,
(SEQ ID NO: 53)
GSGLCSVCQCGGMYSRVVLGQRRWM,
(SEQ ID NO: 54)
GVSPIVHGDAGLLQPRNTRL,
(SEQ ID NO: 55)
CRQRGDGGAMRRGSVCLRGW,
(SEQ ID NO: 56)
GDGTGVQRAAVGFDLTGHSP,
(SEQ ID NO: 57)
AVSACGRCRHCRVPGVPCWLYCVCT,
(SEQ ID NO: 58)
PSTMSIAGGGFCGHPRRGTI,
(SEQ ID NO: 59)
DQCAGHDSVQRHALHGLAVM,
(SEQ ID NO: 60)
GASPCVFRVDSTSGGPLVFPDLQST,
(SEQ ID NO: 61)
RREDSLTRVRQALREALGAG,
and
(SEQ ID NO: 62)
LDVGVAQHRGGTVIRVAMRA,
or a variant thereof.

13. The composition of any one of the above claims, wherein the cellular uptake peptide has an amino acid sequence which is FSFGNSSLRKLPGWRRRRCARGVWR (SEQ ID NO: 49), or a variant thereof.

14. The composition of any of claims 1-11, wherein the cellular uptake peptide is capable of binding to MIA cells and/or has an amino acid sequence selected from:
(SEQ ID NO: 63)
GIRRGGLGRQRGWGRRRKPI,
(SEQ ID NO: 64)
RAPGGFTRTNSVRRATGSM,
(SEQ ID NO: 65)
YGKVGGWRLATVGRRTESRI,
(SEQ ID NO: 66)
KQVGLINGSACSCDMYQRVV,
(SEQ ID NO: 67)
VNSVKGVHRRAFSEQPLGSG,
(SEQ ID NO: 68)
DAGRAPALGWFFFGRRRFGE,
(SEQ ID NO: 69)
GVPGCGSHGADWGREIGATSTGMAL,
(SEQ ID NO: 70)
GVCRWMRPHFRMPRRWRLGR,
(SEQ ID NO: 71)
RSSGMCIIARRHVLSCHSLS,
(SEQ ID NO: 72)
SGVGTCPTRPWVPGVCRQVS,
(SEQ ID NO: 73)
RVIVSGLSGRVRMGVKRRRI,
(SEQ ID NO: 74)
FMRHARPTRGVVRRIGHGV,
(SEQ ID NO: 75)
QLYACLRLVWGRQASAGWRVTMTG,
(SEQ ID NO: 76)
LERIVGQWYSEGLSGDWANL,
(SEQ ID NO: 77)
GASRCFSLCSGSEGAESRRQATTV,
(SEQ ID NO: 78)
HLGCSMPINVFGVCIWKCA,
(SEQ ID NO: 79)
RRLAVRLRVMLRRGPVCIRV,
(SEQ ID NO: 80)
LVDACIGSCQPEGSPPARPPFQLA,
(SEQ ID NO: 81)
RCVGCCKYLIAQAYRFTVAGGAAQYA,
(SEQ ID NO: 82)
AGVHCGLTNVHGVGVRSYRRLYA,
(SEQ ID NO: 83)
VNGWGLGRRGMGIDYRWAVH,
and
(SEQ ID NO: 84)
QSARCAVTMGLDALNLRRGISPVLD,
or a variant thereof.

15. The composition of any of claims 1-11, wherein the cellular uptake peptide is capable of binding to A549 cells and/or has an amino acid sequence selected from:
(SEQ ID NO: 85)
YPWQQVLVPPPGNVHAFATI,
(SEQ ID NO: 86)
TAVAGVGVSAGQLQGGTAAG,
(SEQ ID NO: 87)
FFGLSFPASVAVSDSGVQMR,
(SEQ ID NO: 88)
SSRAVSWMVQLRVQRGSGLV,
(SEQ ID NO: 89)
SRLSLVQSCSVSWGHQTVGT,
(SEQ ID NO: 90)
VARWCQVNWAYGRRLAPPVG,
(SEQ ID NO: 91)
FLLCSLPLTQPPYRVIGAYA,
(SEQ ID NO: 92)
GGPSGIGWQHAWWVFGRQGL,
(SEQ ID NO: 93)
LQRQIAGPRSRQRGRGSRYR,
(SEQ ID NO: 94)
FEDMRLGWRNGQPPGGVAIW,
(SEQ ID NO: 95)
DSWQGARVQPGVGAHTAQQR,
(SEQ ID NO: 96)
GIKTVRGYGVWRWHGGSPRM,
(SEQ ID NO: 97)
IRVWCQWLRTKLAAGQVNCA,
(SEQ ID NO: 98)
SYGVGGRHSGGQGEALVYSV,
(SEQ ID NO: 99)
RAQNCGNWAAVQRINVSSMA,
(SEQ ID NO: 100)
GRAPEQTPCIGWRGGLIKDL,
(SEQ ID NO: 101)
SGSRNFAAARWSHRGFCVPW,
(SEQ ID NO: 102)
IAFSRRGRGWWCQMPGGSRV,
and
(SEQ ID NO: 103)
GCCSAGLPSSLLSTPQSRSR,
or a variant thereof.

16. The composition of claim 2, wherein m is 1, and the cell surface receptor targeting peptide binds to a cancer cell surface receptor or a microvascular endothelial cell surface receptor.

17. The composition of claim 16, wherein the cancer cell surface receptor is insulin-like growth factor type 1 receptor (IGF-1R), epidermal growth factor receptor (EGFR), insulin receptor, GRP78/BiP, or transferrin receptor.

18. The composition of claim 2 or 17, wherein m is 1, and V has an amino acid sequence of DFYGCLLDLSLGVPSGVRRRCITA (SEQ ID NO: 104), or a variant thereof.

19. The composition of claim 2 or 17, wherein m is 1, and V has an amino acid sequence of RPWILHLGRERSTDDQTPVHRMSLMHPLYHTRGKSYKVEG (SEQ ID NO: 105), or a variant thereof.

20. The composition of claim 2, wherein the organelle uptake peptide is a target peptide or signal peptide.

21. The composition of claim 20, wherein the organelle uptake peptide is a nuclear localization signal is a nuclear localization signal, optionally a classical nuclear localization signal.

22. The composition of claim 21, wherein the nuclear localization signal is a monopartite classical nuclear localization signal.

23. The composition of claim 22, wherein the nuclear localization signal has an amino acid sequence of PKKKRKV (SEQ ID NO: 106).

24. The composition of claim 21, wherein the nuclear localization signal is a bipartite classical nuclear localization signal.

25. The composition of claim 21, wherein the nuclear localization signal is a non-classical nuclear localization signal.

26. The composition of claim 2, wherein the organelle uptake peptide is an endoplasmic reticulum localization signal.

27. The composition of claim 2, wherein V or W or X further comprise a cleavage site.

28. The composition of claim 27, wherein the cleavage site is a matrix metalloproteinase cleavage site.

29. The composition of claim 27, wherein the cleavage site is a matrix metalloproteinase 9 cleavage site.

30. The composition of claim 27, wherein the cleavage site has an amino acid sequence of WLPSSI (SEQ ID NO: 107).

31. The composition of claim 27, wherein the cleavage site is a furin cleavage site.

32. The composition of claim 31, wherein the cleavage site has an amino acid sequence of PRRARS (SEQ ID NO: 108).

33. The composition of claim 1, wherein the protein detection peptide is a FLAG tag which has an amino acid sequence of DYKDDDDK (SEQ ID NO: 109).

34. The composition of any one of the above claims, wherein the variant comprises about 1, or about 2, or about 3, or about 4, or about 5 mutations, the mutations selected from substitutions or deletions.

35. The composition of any one of the above claims, wherein the linker is a flexible linker.

36. The composition of claim 35, wherein the linker is substantially comprised of glycine and serine residues.

37. The composition of claim 35 or 36, wherein the linker is (GGS)ₙ, wherein n is 1, or 2, or 3, or 4, or 5.

38. The composition of any one of claims 35-37, wherein the linker is GGSGGSGGSG (SEQ ID NO: 118), or a variant thereof, wherein the variant comprises about 1, or about 2, or about 3, or about 4, or about 5 mutations, the mutations selected from substitutions or deletions.

39. The composition of any one of the above claims, wherein the linker comprises a cleavage site.

40. The composition of claim 39, wherein the cleavage site is a matrix metalloproteinase cleavage site, optionally a matrix metalloproteinase 9 cleavage site, optionally having the amino acid sequence of WLPSSI (SEQ ID NO: 107) or a furin cleavage site, optionally having the amino acid sequence of PRRARS (SEQ ID NO: 108).

41. A composition comprising an amino acid sequence of:
DFYGCLLDLSLGVPSLGWRRRCITADYKDDDDKWLPSSIPKKKRKVDGQKLRDAFTWNMNEKLMTPEMFSEILCDDL D (SEQ ID NO: 110), or an amino acid sequence having at least about 90%, or at least about 95%, or at least about 97%, or at least about 98%, or at least about 99% identity thereof.

42. A composition comprising an amino acid sequence of:
DFYGCLLDLSLGVPSLGWRRRCITAWLPSSIPKKKRKVDGQKLRDAFTWNMNEKLMTPEMFSEILCDDLD (SEQ ID NO: 111), or an amino acid sequence having at least about 90%, or at least about 95%, or at least about 97%, or at least about 98%, or at least about 99% identity thereof.

43. A composition comprising an amino acid sequence of:
QVQLVESGGGVVQPGRSLRLSCAASGGGGSGGGGSWFRQAPGKEREFVAGGGGSGGGGSYADSVKGRFTISRD NSKNTLYLQMNSLRAEDTAVYYCARDFYGCLLDLSLGVPSLGWRRRCITAWGQGLVTVSSGPGGQWLPSSIPKKKR KVDGQKLRDAFTWNMNEKLMTPEMFSEILCDDLD (SEQ ID NO: 112), or an amino acid sequence having at least about 90%, or at least about 95%, or at least about 97%, or at least about 98%, or at least about 99% identity thereof.

44. The compositions of any of claims 1-43, wherein the composition is suitable for administration with one or more additional therapeutic agents.

45. The composition of claim 44, wherein the additional therapeutic agent is tazemetostat.

46. A polynucleotide comprising a nucleic acid sequence encoding the amino acid sequence, or variant thereof of any of the above claims.

47. A vector comprising the polynucleotide of claim 46.

48. A host cell comprising the vector of claim 47.

49. A pharmaceutical composition comprising the composition of any of claims 1-43, and a pharmaceutically acceptable excipient or carrier.

50. A method for treating or preventing a disease, comprising administering an effective amount of the composition of any one of claims 1-43 or the pharmaceutical composition of claim 49 to a patient in need thereof.

51. The method of claim 50, wherein the disease is treatable by targeting a cancer cell surface receptor or a microvascular endothelial cell surface receptor.

52. The method of claim 50, wherein the disease is treatable by targeting tumors, optionally atypical teratoid rhabdoid tumor.

53. The method of any one of claims 50-52, wherein the disease is a cancer optionally selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

54. The method of any one of claims 50-53, wherein the administering is effected using convection-enhanced delivery.

55. A composition comprising a glycoprotein non-metastatic melanoma protein b (GPNMB) recognition domain linked to a therapeutic payload.

56. The composition of claim 55, wherein the GPNMB recognition domain is linked to 1, or 2, or 3, or 4, or 5, or 6, or 7, or 8, or 9, or 10 or more therapeutic payloads.

57. The composition of claim 55 or 56, wherein the GPNMB recognition domain is or comprises,
(a) a single-chain antibody, or fragment thereof comprising CDR1, CDR2, and CDR3, wherein:
CDR1 is or comprises MIHYLGWSKV (SEQ ID NO: 1), or YMGSRQNDMG (SEQ ID NO: 2), or LNRAPYFPNA (SEQ ID NO: 3), or WLSNNFWAW (SEQ ID NO: 4) or a variant thereof, wherein the variant comprises about 1, or about 2, or about 3, or about 4, or about 5 mutations, the mutations selected from substitutions or deletions,
CDR2 is or comprises QIKMWWAMMH (SEQ ID NO: 5) or TSFWIKARKW (SEQ ID NO: 6), or WASWRWFRDH (SEQ ID NO: 7), or QTLKTSWWWT (SEQ ID NO: 8), or a variant thereof, wherein the variant comprises about 1, or about 2, or about 3, or about 4, or about 5 mutations, the mutations selected from substitutions or deletions, and
CDR3 is or comprises LPGGNNFNLYRSFIMTLW (SEQ ID NO: 9) or WQLGSGGLHVWVSCHMYGGVYGKTSELALTWPGMWTRTHLW (SEQ ID NO: 10), or DSPRLDSIWSMSHAMGHL (SEQ ID NO: 11), or YLVRFRLDGVVWHEVVGNVNEYRTAWASIFDWLAKFQLGS (SEQ ID NO: 12), or ILNSCNPTWTRLQFKFCPALLMNGPIFAWFLWAAWVSSAM (SEQ ID NO: 13), or SDEYRFDRSVESVWQDLVIYQSHVLWWTTVHRYLAAWIQW (SEQ ID NO: 14), or VNWINMHSAKWMWWTHGV (SEQ ID NO: 15), or TPTLNYGNFNERDMDTVG (SEQ ID NO: 16), or a variant thereof, wherein the variant comprises about 1, or about 2, or about 3, or about 4, or about 5 mutations, the mutations selected from substitutions or deletions; or
(b) a peptide, the peptide selected from:
LPGGNNFNLYRSFIMTLW (SEQ ID NO: 9), or a variant thereof; WQLGSGGLHVWVSCHMYGGVYGKTSELALTWPGMWTRTHLW (SEQ ID NO: 10), or a variant thereof; DSPRLDSIWSMSHAMGHL (SEQ ID NO: 11), or a variant thereof; YLVRFRLDGWWHEWGNVNEYRTAWASIFDWLAKFQLGS (SEQ ID NO: 12), or a variant thereof; ILNSCNPTWTRLQFKFCPALLMNGPIFAWFLWAAWVSSAM (SEQ ID NO: 13), or a variant thereof; CVLGDYHYLYTGSADHTLRESLSLLTRCRFRRVIYQQQLC (SEQ ID NO: 120), or a variant thereof; FTHISRFGEQHPTLVNLFYPRWQYIFIFTRNALCGQYVWD (SEQ ID NO: 17), or a variant thereof; LSDFEINKRCVLGVFYYLGEGSRGHILSFFHPNVQQFRVP (SEQ ID NO: 18), or a variant thereof; GDFGNRYIQLLVIVLSNGQIFVQKLLSMRPFTINGDRLRQ (SEQ ID NO: 19), or a variant thereof; ASVTLFRLVPMQNAWVTVLPSFAEFVMARPIEERATWGGS (SEQ ID NO: 20), or a variant thereof; WQLGSGGLHVWVSCHMYGGVYGKTSELALTWPGMWTRTHL (SEQ ID NO: 21), or a variant thereof; DSTMTLLARANSHASLLNVYLNINQKVEDVFFMLFLGSPP (SEQ ID NO: 22), or a variant thereof; LATWIQGSKIWSNETYSISIQVSWLSLFVMGQNLYYLLSV (SEQ ID NO: 23), or a variant thereof; SDEYRFDRSVESVWQDLVIYQSHVLWWTTVHRYLAAWIQW (SEQ ID NO: 14), or a variant thereof; VNWINMHSAKWMWWTHGV (SEQ ID NO: 15), or a variant thereof; TPTLNYGNFNERDMDTVG (SEQ ID NO: 16), or a variant thereof; QKVLYTAVPFKHGSYIFCDRIGFIFNYICEFSRSWSTWSM (SEQ ID NO: 24), or a variant thereof; VGNSIQYVKAVQTQYWAAITSWIPITRFTTRVMFLLPQVM (SEQ ID NO: 25), or a variant thereof; QVTARCLQIIVLYKLGNNGVTSLFHVYAMSSVPKFSVKWS (SEQ ID NO: 26), or a variant thereof; GQVCSPFVTSALKLFGADFGWWSLVLMKAVSWVLYITFNA (SEQ ID NO: 27), or a variant thereof; TLDHVRQAYAALSYSTVRCTFVSQMTIFYFGRVMFKALCN (SEQ ID NO: 28), or a variant thereof; AYHLQLEQLRCVSQNVVTSFLDFALTKALALRSVLLGVWM (SEQ ID NO: 29), or a variant thereof; QCFQDDLCVLPVASLLDHTRSMSHLFEGWLPSRSFGFRNG (SEQ ID NO: 30), or a variant thereof; STAWIWQDWFRTFRMAVVSSARDEAVDRVCLAWPARYM (SEQ ID NO: 31), or a variant thereof; LSAADMYKHKIVGEAGEPIFYMVRNWLQKQWPMNFILMSL (SEQ ID NO: 32), or a variant thereof; FLWQWSADSMGAVDACSHKILGLFYHSQNIFARPFLRSYG (SEQ ID NO: 33), or a variant thereof; NIFIPPVGYYLTKQCGTNNFWFYQVLCRRWGADSILFWER (SEQ ID NO: 34), or avariant thereof, wherein the variant comprises about 1, or about 2, or about 3, or about 4, or about 5 mutations, the mutations selected from substitutions or deletions.

58. The composition of claim 57, wherein:
CDR1 is or comprises MIHYLGWSKV (SEQ ID NO: 1),
CDR2 is or comprises QIKMWWAMMH (SEQ ID NO: 5), and
CDR3 is or comprises LPGGNNFNLYRSFIMTLW (SEQ ID NO: 9)
or a variant thereof, wherein the variant comprises about 1, or about 2, or about 3, or about 4, or about 5 mutations, the mutations selected from substitutions or deletions.

59. The composition of claim 57, wherein:
CDR1 is or comprises MIHYLGWSKV (SEQ ID NO: 1),
CDR2 is or comprises QIKMWWAMMH (SEQ ID NO: 5), and
CDR3 is or comprises WQLGSGGLHVWVSCHMYGGVYGKTSELALTWPGMWTRTHLW (SEQ ID NO: 10)
or a variant thereof, wherein the variant comprises about 1, or about 2, or about 3, or about 4, or about 5 mutations, the mutations selected from substitutions or deletions.

60. The composition of claim 57, wherein:
CDR1 is or comprises YMGSRQNDMG (SEQ ID NO: 2),
CDR2 is or comprises TSFWIKARKW (SEQ ID NO: 6), and
CDR3 is or comprises DSPRLDSIWSMSHAMGHL (SEQ ID NO: 11)
or a variant thereof, wherein the variant comprises about 1, or about 2, or about 3, or about 4, or about 5 mutations, the mutations selected from substitutions or deletions.

61. The composition of claim 57, wherein:
CDR1 is or comprises YMGSRQNDMG (SEQ ID NO: 2),
CDR2 is or comprises TSFWIKARKW (SEQ ID NO: 6), and
CDR3 is or comprises YLVRFRLDGVVWHEVVGNVNEYRTAWASIFDWLAKFQLGS (SEQ ID NO: 12)
or a variant thereof, wherein the variant comprises about 1, or about 2, or about 3, or about 4, or about 5 mutations, the mutations selected from substitutions or deletions.

62. The composition of claim 57, wherein:
CDR1 is or comprises LNRAPYFPNA (SEQ ID NO: 3),
CDR2 is or comprises WASWRWFRDH (SEQ ID NO: 7), and
CDR3 is or comprises ILNSCNPTWTRLQFKFCPALLMNGPIFAWFLWAAWVSSAM (SEQ ID NO: 13)
or a variant thereof, wherein the variant comprises about 1, or about 2, or about 3, or about 4, or about 5 mutations, the mutations selected from substitutions or deletions.

63. The composition of claim 57, wherein:
CDR1 is or comprises WLSNNFWAW (SEQ ID NO: 4),
CDR2 is or comprises QTLKTSWWWT (SEQ ID NO: 8), and
CDR3 is or comprises SDEYRFDRSVESVWQDLVIYQSHVLWWTTVHRYLAAWIQW (SEQ ID NO: 14)
or a variant thereof, wherein the variant comprises about 1, or about 2, or about 3, or about 4, or about 5 mutations, the mutations selected from substitutions or deletions.

64. The composition of claim 57, wherein:
CDR1 is or comprises LNRAPYFPNA (SEQ ID NO: 3),
CDR2 is or comprises WASWRWFRDH (SEQ ID NO: 7), and
CDR3 is or comprises VNWINMHSAKWMWWTHGV (SEQ ID NO: 15)
or a variant thereof, wherein the variant comprises about 1, or about 2, or about 3, or about 4, or about 5 mutations, the mutations selected from substitutions or deletions.

65. The composition of claim 57, wherein:
CDR1 is or comprises WLSNNFWAW (SEQ ID NO: 4),
CDR2 is or comprises QTLKTSWWWT (SEQ ID NO: 8), and
CDR3 is or comprises TPTLNYGNFNERDMDTVG (SEQ ID NO: 16)
or a variant thereof, wherein the variant comprises about 1, or about 2, or about 3, or about 4, or about 5 mutations, the mutations selected from substitutions or deletions.

66. The composition of any one of claims 57-65, wherein the single-chain antibody, or fragment thereof, further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

67. The composition of any one of claims 57-66, wherein the variable region FW sequences are human.

68. The composition of claims 57 or 58, wherein the single-chain antibody comprises an amino acid sequence of:
MAQVQLVESGGGVVQPGRSLRLSCAASMIHYLGWSKVWFRQAPGKEREFVAQKMWWAMMHYADSVKGRFTISR DNSKNTLYLQMNSLRAEDTAVYYCARLPGGNNFNLYRSFIMTLWWGQGTLVTVSSGPGGQ (SEQ ID NO: 35), or an amino acid sequence having at least about 90%, or at least about 95%, or at least about 97%, or at least about 98%, or at least about 99% identity thereto.

69. The composition of claims 57 or 59, wherein the single-chain antibody comprises an amino acid sequence of:
MAQVQLVESGGGVVQPGRSLRLSCAASMIHYLGWSKVWFRQAPGKEREFVAQKMWWAMMHYADSVKGRFTISR DNSKNTLYLQMNSLRAEDTAVYYCARWQLGSGGLHVWVSCHMYGGVYGKTSELALTWPGMWTRTHLWWGQGTL VTVSSGPGGQ (SEQ ID NO: 36), or an amino acid sequence having at least about 90%, or at least about 95%, or at least about 97%, or at least about 98%, or at least about 99% identity thereto.

70. The composition of claims 57 or 60, wherein the single-chain antibody comprises an amino acid sequence of:
MAQVQLVESGGGVVQPGRSLRLSCAASYMGSRQNDMGWFRQAPGKEREFVATSFWIKARKWYADSVKGRFTISR DNSKNTLYLQMNSLRAEDTAVYYCARDSPRLDSIWSMSHAMGHLWGQGTLVTVSSGPGGQ (SEQ ID NO: 37), or an amino acid sequence having at least about 90%, or at least about 95%, or at least about 97%, or at least about 98%, or at least about 99% identity thereto.

71. The composition of claims 55 or 61, wherein the single-chain antibody comprises an amino acid sequence of:
MAQVQLVESGGGVVQPGRSLRLSCAASYMGSRQNDMGWFRQAPGKEREFVATSFWIKARKWYADSVKGRFTISR DNSKNTLYLQMNSLRAEDTAVYYCARYLVRFRLDGVVWHEVVGNVNEYRTAWASIFDWLAKFQLGSWGQGTLVTV SSGPGGQ (SEQ ID NO: 38), or an amino acid sequence having at least about 90%, or at least about 95%, or at least about 97%, or at least about 98%, or at least about 99% identity thereto.

72. The composition of claims 55 or 62, wherein the single-chain antibody comprises an amino acid sequence of:
MQVQLVESGGGVVQPGRSLRLSCAASLNRAPYFPNAWFRQAPGKEREFVAWASWRWFRDHYADSVKGRFTISRD NSKNTLYLQMNSLRAEDTAVYYCARILNSCNPTWTRLQFKFCPALLMNGPIFAWFLWAAWVSSAMWGQGTLVTVSS GPGGQ (SEQ ID NO: 39), or an amino acid sequence having at least about 90%, or at least about 95%, or at least about 97%, or at least about 98%, or at least about 99% identity thereto.

73. The composition of claims 55 or 63, wherein the single-chain antibody comprises an amino acid sequence of:
MQVQLVESGGGVVQPGRSLRLSCAASSWLSNNFWAWWFRQAPGKEREFVAQTLKTSWWWTYADSVKGRFTISR DNSKNTLYLQMNSLRAEDTAVYYCARSDEYRFDRSVESVWQDLVIYQSHVLWWTTVHRYLAAWIQWWGQGTLVTV SSGPGGQ (SEQ ID NO: 40), or an amino acid sequence having at least about 90%, or at least about 95%, or at least about 97%, or at least about 98%, or at least about 99% identity thereto.

74. The composition of claims 55 or 64, wherein the single-chain antibody comprises an amino acid sequence of:
MQVQLVESGGGVVQPGRSLRLSCAASLNRAPYFPNAWFRQAPGKEREFVAWASWRWFRDHYADSVKGRFTISRD NSKNTLYLQMNSLRAEDTAVYYCARVNWINMHSAKWMWWTHGVWGQGTLVTVSSGPGGQ (SEQ ID NO: 41), or an amino acid sequence having at least about 90%, or at least about 95%, or at least about 97%, or at least about 98%, or at least about 99% identity thereto.

75. The composition of claims 55 or 65, wherein the single-chain antibody comprises an amino acid sequence of:
MQVQLVESGGGVVQPGRSLRLSCAASSWLSNNFWAWWFRQAPGKEREFVAQTLKTSWWWTYADSVKGRFTISR DNSKNTLYLQMNSLRAEDTAVYYCARTPTLNYGNFNERDMDTVGWGQGTLVTVSSGPGGQ (SEQ ID NO: 42), or an amino acid sequence having at least about 90%, or at least about 95%, or at least about 97%, or at least about 98%, or at least about 99% identity thereto.

76. The composition of claim 56, wherein the therapeutic payload is an anticancer agent.

77. The composition of claim 56 or 76, wherein the therapeutic payload is a small molecule drug, biological, or radionuclide.

78. The composition of claim 77, wherein the therapeutic payload is a biological, which is selected from a toxin, an antibody, or an oligonucleotide.

79. The composition of claim 76, wherein the anticancer agent is monomethyl auristatin E, vetodin monomethyl auristatin F, or mafodotin.

80. The composition of claim 76, wherein the anticancer agent is a histone deacetylase (HDAC) inhibitor.

81. The composition of claim 80, wherein the HDAC inhibitor is selected from vorinostat, panobinostat, tazemetostat, mocetinostat, romidepsin, trichostatin A and trapoxin A.

82. The composition of claim 77, wherein the therapeutic payload is gemcitabine, cisplatin, or an antisense oligonucleotide that binds to kras.

83. The composition of claim 77, wherein the therapeutic payload is a SmarcB1 mimic.

84. The composition of any one of claims 56-83, wherein the GPNMB recognition domain and therapeutic payload are joined with a linker which is optionally substantially comprised of glycine and serine residues.

85. The composition of claim 84, wherein the linker is (GGS)ₙ, wherein n is 1, or 2, or 3, or 4, or 5.

86. The composition of claim 85, wherein the linker is GGSGGSGGSG (SEQ ID NO: 118), or a variant thereof, wherein the variant comprises about 1, or about 2, or about 3, or about 4, or about 5 mutations, the mutations selected from substitutions or deletions.

87. The composition of any one of claims 56-86, wherein the linker comprises a cleavage site.

88. The composition of claim 87, wherein the cleavage site is a matrix metalloproteinase cleavage site, optionally a matrix metalloproteinase 9 cleavage site, optionally having the amino acid sequence of WLPSSI (SEQ ID NO: 107) or a furin cleavage site, optionally having the amino acid sequence of PRRARS (SEQ ID NO: 108).

89. The composition of any one of claims 56-88, wherein the composition binds to GPNMB expressed on a cell.

90. The composition of any one of claims 56-89, wherein the composition reduces, abrogates, or disrupts expression of GPNMB on a cell, optionally a cancer stem cell.

91. A polynucleotide comprising a nucleic acid sequence encoding the amino acid sequence, or variant thereof of any of claims 56-90.

92. A composition comprising a vector composition comprising the polynucleotide of claim 91.

93. A composition comprising a host cell comprising the polynucleotide of claim 91 or the vector of claim 92.

94. A pharmaceutical composition comprising the composition of any one of claims 56-93, and a pharmaceutically acceptable excipient or carrier.

95. A method of treating or preventing cancer, comprising administering an effective amount of the composition of any one of claims 56-93 or the pharmaceutical composition of claim 94 to a subject in need thereof.

96. The method of claim 95, wherein the cancer is triple negative breast cancer.

97. The method of claim 95, wherein the cancer is a solid tumor.

98. The method of claim 95, wherein the cancer is a blood cancer.

99. The method of any one of claims 95-98, wherein the cancer is selected form one or more of a cancer of a blood vessel, an eye tumor, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.
